(19) United States
(12) Patent Application Publication
(10) Pub. No.: US 2004/0248157 A1
(43) Pub. Date: Dec. 9, 2004

(54) Novel polynucleotides encoding soluble polypeptides and methods using same

(76) Inventors: Michal Ayalon-Soffer, Ramat HaSharon (IL); Sarah Pollock, Tel Aviv (IL); Alex Diber, Herzlia (IL); Zurit Levine, Herzlia (IL); Sergey Nemzer, Ramat Gan (IL); Dvir Dahary, Tel Aviv (IL); Rotem Sorek, Rehovot (IL); Erez Levanon, Petach Tikva (IL); Galit Rotman, Herzlia (IL); Kinneret Savitsky, Tel Aviv (IL); Chen Chermesh, Mishmar HaShiva (IL); Liat Mintz, East Brunswick, NJ (US); Shiri Freilich, Haifa (IL); Nili Beck, Kfar Saba (IL); Wei-Yong Zhu, Plainsboro, NJ (US); Alon Wasserman, New York, NY (US); Idit Azar, Tel Aviv (IL); Jeanne Bernstein, Kfar Yona (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 10/764,833

(22) Filed: Jan. 27, 2004

Related U.S. Application Data
(63) Continuation-in-part of application No. 10/426,002, filed on Apr. 30, 2003.
(63) Continuation-in-part of application No. 10/242,799, filed on Sep. 13, 2002.
(60) Provisional application No. 60/322,285, filed on Sep. 14, 2001.
(60) Provisional application No. 60/322,359, filed on Sep. 14, 2001.
(60) Provisional application No. 60/322,506, filed on Sep. 14, 2001.
(60) Provisional application No. 60/324,524, filed on Sep. 26, 2001.
(60) Provisional application No. 60/354,242, filed on Feb. 6, 2002.
(60) Provisional application No. 60/371,494, filed on Apr. 11, 2002.
(60) Provisional application No. 60/384,096, filed on May 31, 2002.
(60) Provisional application No. 60/397,784, filed on Jul. 24, 2002.

(51) Int. Cl. C12Q 1/68; C07H 21/04

(52) U.S. Cl. 435/6; 435/69.1; 435/320.1; 435/325; 530/324; 536/23.5

(57) ABSTRACT

An isolated polynucleotide is provided. The isolated polynucleotide comprising a nucleic acid sequence encoding a polypeptide having an amino acid sequence at least 70% identical to SEQ ID NO: 1, as determined using the LALIGN software of EMBnet Switzerland (http://www.ch.embnet.org/index.html) using default parameters

This application claims the benefit of priority from U.S. Provisional Patent Application No. 60/322,285, filed Sep. 14, 2001; 60/322,359, filed Sep. 14, 2001; 60/322,506, filed Sep. 14, 2001; 60/324,524, filed Sep. 26, 2001; 60/354,242, filed Feb. 6, 2002; 60/371,494, filed Apr. 11, 2002; 60/384,096, filed May 31, 2002; 60/397,784, filed Jul. 24, 2002; and is a continuation in part of U.S. patent application Ser. Nos. 10/242,799, filed Sep. 13, 2002; and Ser. No. 10/426,002, filed Apr. 30, 2003.

### FIELD AND BACKGROUND OF THE INVENTION

The present invention relates to novel soluble polypeptides and polynucleotides encoding same and more particularly, to therapeutic and diagnostic methods and kits utilizing same.

Extracellular proteins including receptors and their corresponding ligands play active roles in the formation, differentiation and maintenance of multicellular organisms. Any fate of an individual cell including proliferation, migration, differentiation, or interaction with other cells, is typically governed by information received from distant cells and/or the immediate environment. This information is often transmitted by secreted polypeptides such as, mitogenic factors, survival factors, cytotoxic factors, differentiation factors, neuropeptides, and hormones, which are, in turn, received and interpreted by diverse cell receptors or membrane-bound proteins. These secreted polypeptides or signaling molecules are normally transferred through the cellular secretory pathway to reach their site of action at the extracellular environment.

Secreted proteins have various industrial applications, including as pharmaceuticals, diagnostics, biosensors and bioreactors. Most protein drugs available to date, including thrombolytic polypeptide sequences, interferons, interleukins, erythropoietins, colony stimulating factors, and various other cytokines, are secretory proteins. Their receptors, which are membrane proteins, also have potential as therapeutic or diagnostic polynucleotide or polypeptide sequences. For example, receptor immunoadhesins, can be employed as therapeutic polynucleotide or polypeptide sequences to block receptor-ligand interactions. The membrane-bound proteins can also be employed for screening of potential peptide or small molecule inhibitors of the relevant receptor/ligand interaction.

For these reasons, efforts are being made by both industry and academia to identify new, native, membrane-bound or secreted proteins. Many efforts are focused on the screening of mammalian recombinant DNA libraries to identify the coding sequences for such proteins. Examples of such screening methods and techniques are described in, for example, Klein et al., Proc. Natl. Acad. Sci. 93:7108-7113 (1996); U.S. Pat. No. 5,536,637

The present inventors have previously designed algorithms, which allow for the mass prediction of yet unknown gene products and for annotating these [see U.S. Pat. No. 6,625,545; U.S. patent application Ser. No. 10/426,002; a U.S. patent application entitled METHODS AND SYSTEMS FOR ANNOTATING BIOMOLECULAR SEQUENCES (Attorney Docket No. 26940), filed concurrently herewith, assigned to the same assignee hereof and contains subject matter related, in certain respects, to the subject matter of the instant application, the teachings of all of which are incorporated herein by reference; and Example 1 of the Examples section which follows].

While applying the above-mentioned algorithms the present inventors uncovered novel naturally occurring variants of extracellular gene products, which as described above, play pivotal roles in disease onset and progression. As such these variants can be used in the diagnosis and therapy of a wide range of diseases.

### SUMMARY OF THE INVENTION

According to one aspect of the present invention there is provided an isolated polynucleotide comprising a nucleic acid sequence encoding a polypeptide having an amino acid sequence at least 70% identical to SEQ ID NO: 1, as determined using the LALIGN software of EMBnet Switzerland (http://www.ch.embnet.org/index.html) using default parameters.

According to further features in preferred embodiments of the invention described below, the nucleic acid sequence is as set forth in SEQ ID NO: 3 or 4.

According to still further features in the described preferred embodiments the polypeptide is as set forth in SEQ ID NO: 1 or 2.

According to another aspect of the present invention there is provided an isolated polynucleotide as set forth in SEQ ID NO: 3 or 4.

According to yet another aspect of the present invention there is provided an isolated polypeptide as set forth in SEQ ID NO: 1 or 2.

According to still another aspect of the present invention there is provided a nucleic acid construct comprising any of the isolated polynucleotide of the present invention.

According to still further features in the described preferred embodiments the nucleic acid construct further comprising a promoter for regulating transcription of the isolated polynucleotide in sense or antisense orientation.

According to still further features in the described preferred embodiments the nucleic acid construct further comprising a positive and a negative selection markers for selecting for homologous recombination events.

According to an additional aspect of the present invention there is provided a host cell comprising the nucleic acid construct.

According to yet an additional aspect of the present invention there is provided An isolated polypeptide comprising an amino acid sequence at least 70% identical to SEQ ID NO: 1, as determined using the LALIGN software of EMBnet Switzerland (http://www.ch.embnet.org/index.html) using default parameters or an active portion thereof.

According to still an additional aspect of the present invention there is provided An antibody or an antibody fragment being capable of specifically binding a polypeptide having an amino acid sequence at least 70% identical to SEQ ID NO: 1, as determined using the LALIGN software of EMBnet Switzerland (http://www.ch.embnet.org/index.html) using default parameters.

According to a further aspect of the present invention there is provided an oligonucleotide specifically hybridizable with a nucleic acid sequence encoding a polypeptide having an amino acid at least 70% identical to SEQ ID NO: 1, as determined using the LALIGN software of EMBnet Switzerland (http://www.ch.embnet.org/index.html) using default parameters.

According to yet a further aspect of the present invention there is provided A pharmaceutical composition comprising a therapeutically effective amount of a polypeptide having an amino acid sequence at least 70% identical to SEQ ID NO: 1, as determined using the LALIGN software of EMBnet Switzerland (http://www.ch.embnet.org/index.html) using default parameters and a pharmaceutically acceptable carrier or diluent.

According to still a further aspect of the present invention there is provided A method of treating Met-related disease in a subject, the method comprising upregulating in the subject expression of a polypeptide having an amino acid sequence at least 70% identical to SEQ ID NO: 1 as determined using the LALIGN software of EMBnet Switzerland (http://www.ch.embnet.org/index.html) using default parameters, thereby treating the Met-related disease in a subject.

According to still a further aspect of the present invention there is provided An isolated polynucleotide comprising a nucleic acid sequence encoding a polypeptide having an amino acid sequence at least 75% identical to SEQ ID NO: 5, as determined using the LALIGN software of EMBnet Switzerland (http://www.ch.embnet.org/index.html) using default parameters.

According to still further features in the described preferred embodiments the nucleic acid sequence is as set forth in SEQ ID NO: 7 or 8.

According to still further features in the described preferred embodiments the polypeptide is as set forth in SEQ ID NO: 5 or 6.

According to still a further aspect of the present invention there is provided an isolated polynucleotide as set forth in SEQ ID NO: 7 or 8.

According to still a further aspect of the present invention there is provided an isolated polypeptide as set forth in SEQ ID NO: 5 or 6.

According to still a further aspect of the present invention there is provided an isolated polypeptide comprising an amino acid sequence at least 75% identical to SEQ ID NO: 5, as determined using the LALIGN software of EMBnet Switzerland (http://www.ch.embnet.org/index.html) using default parameters or an active portion thereof.

According to still a further aspect of the present invention there is provided an antibody or an antibody fragment being capable of binding a polypeptide having an amino acid sequence at least 75% identical to SEQ ID NO: 5, as determined using the LALIGN software of EMBnet Switzerland (http://www.ch.embnet.org/index.html) using default parameters.

According to still a further aspect of the present invention there is provided an oligonucleotide hybridizable with a nucleic acid sequence encoding a polypeptide having an amino acid at least 75% identical to SEQ ID NO: 5, as determined using the LALIGN software of EMBnet Switzerland (http://www.ch.embnet.org/index.html) using default parameters.

According to still a further aspect of the present invention there is provided a pharmaceutical composition comprising a therapeutically effective amount of a polypeptide having an amino acid sequence at least 75% identical to SEQ ID NO: 5, as determined using the LALIGN software of EMBnet Switzerland (http://www.ch.embnet.org/index.html) using default parameters and a pharmaceutically acceptable carrier or diluent.

According to still a further aspect of the present invention there is provided a method of treating an IL-6-related disease in a subject, the method comprising upregulating in the subject expression of a polypeptide having an amino acid sequence at least 75% identical to SEQ ID NO: 5 as determined using the LALIGN software of EMBnet Switzerland (http://www.ch.embnet.org/index.html) using default parameters, thereby treating the IL-6-related disease in the subject.

According to still a further aspect of the present invention there is provided an isolated polynucleotide comprising a nucleic acid sequence encoding a polypeptide having an amino acid sequence at least 85% identical to SEQ ID NO: 9, as determined using the LALIGN software of EMBnet Switzerland (http://www.ch.embnet.org/index.html) using default parameters.

According to still further features in the described preferred embodiments the nucleic acid sequence is as set forth in SEQ ID NO: 11 or 12.

According to still further features in the described preferred embodiments the polypeptide is as set forth in SEQ ID NO: 9 or 10.

According to still a further aspect of the present invention there is provided an isolated polynucleotide as set forth in SEQ ID NO: 11 or 12.

According to still a further aspect of the present invention there is provided an isolated polypeptide as set forth in SEQ ID NO: 9 or 10.

According to still a further aspect of the present invention there is provided an isolated polypeptide comprising an amino acid sequence at least 85% identical to SEQ ID NO: 9, as determined using the LALIGN software of EMBnet Switzerland (http://www.ch.embnet.org/index.html) using default parameters or an active portion thereof.

According to still a further aspect of the present invention there is provided an antibody or an antibody fragment being capable of binding a polypeptide having an amino acid sequence at least 85% identical to SEQ ID NO: 9, as determined using the LALIGN software of EMBnet Switzerland (http://www.ch.embnet.org/index.html) using default parameters.

According to still a further aspect of the present invention there is provided an oligonucleotide hybridizable with a nucleic acid sequence encoding a polypeptide having an amino acid at least 85% identical to SEQ ID NO: 9, as determined using the LALIGN software of EMBnet Switzerland (http://www.ch.embnet.org/index.html) using default parameters.

According to still a further aspect of the present invention there is provided a pharmaceutical composition comprising a therapeutically effective amount of a IL-7 polypeptide having an amino acid sequence at least 85% identical to SEQ ID NO: 9, as determined using the LALIGN software of EMBnet Switzerland (http://www.ch.embnet.org/index.html) using default parameters and a pharmaceutically acceptable carrier or diluent.

According to still a further aspect of the present invention there is provided a method of treating IL-7-related disease in a subject, the method comprising upregulating in the subject expression of a polypeptide having an amino acid sequence at least 85% identical to SEQ ID NO: 9 as determined using the LALIGN software of EMBnet Switzerland (http://www.ch.embnet.org/index.html) using default parameters.

According to still a further aspect of the present invention there is provided an isolated polynucleotide comprising a nucleic acid sequence encoding a polypeptide having an amino acid sequence at least 85% identical to SEQ ID NO: 13, as determined using the LALIGN software of EMBnet Switzerland (http://www.ch.embnet.org/index.html) using default parameters.

According to still further features in the described preferred embodiments the nucleic acid sequence is as set forth in SEQ ID NO: 15 or 16.

According to still further features in the described preferred embodiments the polypeptide is as set forth in SEQ ID NO: 13 or 14.

According to still a further aspect of the present invention there is provided an isolated polynucleotide as set forth in SEQ ID NO: 15 or 16.

According to still a further aspect of the present invention there is provided an isolated polypeptide as set forth in SEQ ID NO: 13 or 14.

According to still a further aspect of the present invention there is provided an isolated polypeptide comprising an amino acid sequence at least 85% identical to SEQ ID NO: 13, as determined using the LALIGN software of EMBnet Switzerland (http://www.ch.embnet.org/index.html) using default parameters or an active portion thereof.

According to still a further aspect of the present invention there is provided an antibody or an antibody fragment being capable of binding a polypeptide having an amino acid sequence at least 85% identical to SEQ ID NO: 13, as determined using the LALIGN software of EMBnet Switzerland (http://www.ch.embnet.org/index.html) using default parameters.

According to still a further aspect of the present invention there is provided an oligonucleotide hybridizable with a nucleic acid sequence encoding a polypeptide having an amino acid at least 85% identical to SEQ ID NO: 13, as determined using the LALIGN software of EMBnet Switzerland (http://www.ch.embnet.org/index.html) using default parameters.

According to still a further aspect of the present invention there is provided a pharmaceutical composition comprising a therapeutically effective amount of a polypeptide having an amino acid sequence at least 85% identical to SEQ ID NO: 13, as determined using the LALIGN software of EMBnet Switzerland (http://www.ch.embnet.org/index.html) using default parameters and a pharmaceutically acceptable carrier or diluent.

According to still a further aspect of the present invention there is provided a method of treating IL-7-related disease in a subject, the method comprising upregulating in the subject expression of a polypeptide having an amino acid sequence at least 85% identical to SEQ ID NO: 13 as determined using the LALIGN software of EMBnet Switzerland (http://www.ch.embnet.org/index.html) using default parameters.

According to still a further aspect of the present invention there is provided an isolated polynucleotide comprising a nucleic acid sequence encoding a polypeptide having an amino acid sequence at least 60% identical to SEQ ID NO: 17, as determined using the LALIGN software of EMBnet Switzerland (http://www.ch.embnet.org/index.html) using default parameters.

According to still further features in the described preferred embodiments the nucleic acid sequence is as set forth in SEQ ID NO: 19 or 20.

According to still further features in the described preferred embodiments the polypeptide is as set forth in SEQ ID NO: 17 or 18.

According to still a further aspect of the present invention there is provided an isolated polynucleotide as set forth in SEQ ID NO: 19 or 20.

According to still a further aspect of the present invention there is provided an isolated polypeptide as set forth in SEQ ID NO: 17 or 18.

According to still a further aspect of the present invention there is provided an isolated polypeptide comprising an amino acid sequence at least 60% identical to SEQ ID NO: 17, as determined using the LALIGN software of EMBnet Switzerland (http://www.ch.embnet.org/index.html) using default parameters or an active portion thereof.

According to still a further aspect of the present invention there is provided an antibody or an antibody fragment being capable of binding a polypeptide having an amino acid sequence at least 60% identical to SEQ ID NO: 17, as determined using the LALIGN software of EMBnet Switzerland (http://www.ch.embnet.org/index.html) using default parameters.

According to still a further aspect of the present invention there is provided an oligonucleotide hybridizable with a nucleic acid sequence encoding a polypeptide having an amino acid at least 60% identical to SEQ ID NO: 17, as determined using the LALIGN software of EMBnet Switzerland (http://www.ch.embnet.org/index.html) using default parameters.

According to still a further aspect of the present invention there is provided a pharmaceutical composition comprising a therapeutically effective amount of a polypeptide having an amino acid sequence at least 60% identical to SEQ ID NO: 17, as determined using the LALIGN software of EMBnet Switzerland (http://www.ch.embnet.org/index.html) using default parameters and a pharmaceutically acceptable carrier or diluent.

According to still a further aspect of the present invention there is provided a method of treating TNFR9-related disease in a subject, the method comprising upregulating in the subject expression of a polypeptide having an amino acid sequence at least 60% identical to SEQ ID NO: 17 as determined using the LALIGN software of EMBnet Switzerland (http://www.ch.embnet.org/index.html) using default parameters.

According to still a further aspect of the present invention there is provided an isolated polynucleotide comprising a nucleic acid sequence encoding a polypeptide having an amino acid sequence at least 50% identical to SEQ ID NO: 25, as determined using the LALIGN software of EMBnet Switzerland (http://www.ch.embnet.org/index.html) using default parameters.

According to still further features in the described preferred embodiments the nucleic acid sequence is as set forth in SEQ ID NO: 27 or 28.

According to still further features in the described preferred embodiments the polypeptide is as set forth in SEQ ID NO: 25 or 26.

According to still a further aspect of the present invention there is provided an isolated polynucleotide as set forth in SEQ ID NO: 27 or 28.

According to still a further aspect of the present invention there is provided an isolated polypeptide as set forth in SEQ ID NO: 25 or 26.

According to still a further aspect of the present invention there is provided an isolated polypeptide comprising an amino acid sequence at least 50% identical to SEQ ID NO: 25, as determined using the LALIGN software of EMBnet Switzerland (http://www.ch.embnet.org/index.html) using default parameters or an active portion thereof.

According to still a further aspect of the present invention there is provided an antibody or an antibody fragment being capable of binding a polypeptide having an amino acid sequence at least 50% identical to SEQ ID NO: 25, as determined using the LALIGN software of EMBnet Switzerland (http://www.ch.embnet.org/index.html) using default parameters.

According to still a further aspect of the present invention there is provided an oligonucleotide hybridizable with a nucleic acid sequence encoding a polypeptide having an amino acid at least 50% identical to SEQ ID NO: 25, as determined using the LALIGN software of EMBnet Switzerland (http://www.ch.embnet.org/index.html) using default parameters.

According to still a further aspect of the present invention there is provided a pharmaceutical composition comprising a therapeutically effective amount of a polypeptide having an amino acid sequence at least 50% identical to SEQ ID NO: 25, as determined using the LALIGN software of EMBnet Switzerland (http://www.ch.embnet.org/index.html) using default parameters and a pharmaceutically acceptable carrier or diluent.

According to still a further aspect of the present invention there is provided a method of treating IL-4R-related disease in a subject, the method comprising upregulating in the subject expression of a polypeptide having an amino acid sequence at least 50% identical to SEQ ID NO: 25 as determined using the LALIGN software of EMBnet Switzerland (http://www.ch.embnet.org/index.html) using default parameters.

According to still a further aspect of the present invention there is provided an isolated polynucleotide comprising a nucleic acid sequence encoding a polypeptide having an amino acid sequence at least 50% identical to SEQ ID NO: 21, as determined using the LALIGN software of EMBnet Switzerland (http://www.ch.embnet.org/index.html) using default parameters.

According to still further features in the described preferred embodiments the nucleic acid sequence is as set forth in SEQ ID NO: 23 or 24.

According to still further features in the described preferred embodiments the polypeptide is as set forth in SEQ ID NO: 21 or 22.

According to still a further aspect of the present invention there is provided an isolated polynucleotide as set forth in SEQ ID NO: 23 or 24.

According to still a further aspect of the present invention there is provided an isolated polypeptide as set forth in SEQ ID NO: 21 or 22.

According to still a further aspect of the present invention there is provided an isolated polypeptide comprising an amino acid sequence at least 50% identical to SEQ ID NO: 21, as determined using the LALIGN software of EMBnet Switzerland (http://www.ch.embnet.org/index.html) using default parameters or an active portion thereof.

According to still a further aspect of the present invention there is provided an antibody or an antibody fragment being capable of binding a polypeptide having an amino acid sequence at least 50% identical to SEQ ID NO: 21, as determined using the LALIGN software of EMBnet Switzerland (http://www.ch.embnet.org/index.html) using default parameters.

According to still a further aspect of the present invention there is provided an oligonucleotide hybridizable with a nucleic acid sequence encoding a polypeptide having an amino acid at least 50% identical to SEQ ID NO: 21, as determined using the LALIGN software of EMBnet Switzerland (http://www.ch.embnet.org/index.html) using default parameters.

According to still a further aspect of the present invention there is provided a pharmaceutical composition comprising a therapeutically effective amount of a polypeptide having an amino acid sequence at least 50% identical to SEQ ID NO: 21, as determined using the LALIGN software of EMBnet Switzerland (http://www.ch.embnet.org/index.html) using default parameters and a pharmaceutically acceptable carrier or diluent.

According to still a further aspect of the present invention there is provided a method of treating IL-4R-related disease in a subject, the method comprising upregulating in the subject expression of a polypeptide having an amino acid sequence at least 50% identical to SEQ ID NO: 21 as determined using the LALIGN software of EMBnet Switzerland (http://www.ch.embnet.org/index.html) using default parameters.

According to still a further aspect of the present invention there is provided an isolated polynucleotide comprising a nucleic acid sequence encoding a polypeptide having an amino acid sequence at least 50% identical to SEQ ID NO: 29, as determined using the LALIGN software of EMBnet Switzerland (http://www.ch.embnet.org/index.html) using default parameters.

According to still further features in the described preferred embodiments the nucleic acid sequence is as set forth in SEQ ID NO: 31 or 32.

According to still further features in the described preferred embodiments the polypeptide is as set forth in SEQ ID NO: 29 or 30.

According to still a further aspect of the present invention there is provided an isolated polynucleotide as set forth in SEQ ID NO: 31 or 32.

According to still a further aspect of the present invention there is provided an isolated polypeptide as set forth in SEQ ID NO: 29 or 30.

According to still a further aspect of the present invention there is provided an isolated polypeptide comprising an amino acid sequence at least 50% identical to SEQ ID NO: 29, as determined using the LALIGN software of EMBnet Switzerland (http://www.ch.embnet.org/index.html) using default parameters or an active portion thereof.

According to still a further aspect of the present invention there is provided an antibody or an antibody fragment being capable of binding a polypeptide having an amino acid sequence at least 50% identical to SEQ ID NO: 29, as determined using the LALIGN software of EMBnet Switzerland (http://www.ch.embnet.org/index.html) using default parameters.

According to still a further aspect of the present invention there is provided an oligonucleotide hybridizable with a nucleic acid sequence encoding a polypeptide having an amino acid at least 50% identical to SEQ ID NO: 29, as determined using the LALIGN software of EMBnet Switzerland (http://www.ch.embnet.org/index.html) using default parameters.

According to still a further aspect of the present invention there is provided a pharmaceutical composition comprising a therapeutically effective amount of a polypeptide having an amino acid sequence at least 50% identical to SEQ ID NO: 29, as determined using the LALIGN software of EMBnet Switzerland (http://www.ch.embnet.org/index.html) using default parameters and a pharmaceutically acceptable carrier or diluent.

According to still a further aspect of the present invention there is provided a method of treating TGR2-related disease in a subject, the method comprising upregulating in the subject expression of a polypeptide having an amino acid sequence at least 50% identical to SEQ ID NO: 29 as determined using the LALIGN software of EMBnet Switzerland (http://www.ch.embnet.org/index.html) using default parameters.

According to still a further aspect of the present invention there is provided an isolated polynucleotide comprising a nucleic acid sequence encoding a polypeptide having an amino acid sequence at least 80% identical to SEQ ID NO: 33, as determined using the LALIGN software of EMBnet Switzerland (http://www.ch.embnet.org/index.html) using default parameters.

According to still further features in the described preferred embodiments the nucleic acid sequence is as set forth in SEQ ID NO: 35 or 36.

According to still further features in the described preferred embodiments the polypeptide is as set forth in SEQ ID NO: 33 or 34.

According to still a further aspect of the present invention there is provided an isolated polynucleotide as set forth in SEQ ID NO: 35 or 36.

According to still a further aspect of the present invention there is provided an isolated polypeptide as set forth in SEQ ID NO: 33 or 34.

According to still a further aspect of the present invention there is provided an isolated polypeptide comprising an amino acid sequence at least 80% identical to SEQ ID NO: 33, as determined using the LALIGN software of EMBnet Switzerland (http://www.ch.embnet.org/index.html) using default parameters or an active portion thereof.

According to still a further aspect of the present invention there is provided an antibody or an antibody fragment being capable of binding a polypeptide having an amino acid sequence at least 80% identical to SEQ ID NO: 33, as determined using the LALIGN software of EMBnet Switzerland (http://www.ch.embnet.org/index.html) using default parameters.

According to still a further aspect of the present invention there is provided an oligonucleotide hybridizable with a nucleic acid sequence encoding a polypeptide having an amino acid at least 80% identical to SEQ ID NO: 33, as determined using the LALIGN software of EMBnet Switzerland (http://www.ch.embnet.org/index.html) using default parameters.

According to still a further aspect of the present invention there is provided a pharmaceutical composition comprising a therapeutically effective amount of a polypeptide having an amino acid sequence at least 80% identical to SEQ ID NO: 33, as determined using the LALIGN software of EMBnet Switzerland (http://www.ch.embnet.org/index.html) using default parameters and a pharmaceutically acceptable carrier or diluent.

According to still a further aspect of the present invention there is provided a method of treating ITAV-related disease in a subject, the method comprising upregulating in the subject expression of a polypeptide having an amino acid sequence at least 80% identical to SEQ ID NO: 33 as determined using the LALIGN software of EMBnet Switzerland (http://www.ch.embnet.org/index.html) using default parameters.

According to still a further aspect of the present invention there is provided an isolated polynucleotide comprising a nucleic acid sequence encoding a polypeptide having an amino acid sequence at least 70% identical to SEQ ID NO: 37, as determined using the LALIGN software of EMBnet Switzerland (http://www.ch.embnet.org/index.html) using default parameters.

According to still further features in the described preferred embodiments the nucleic acid sequence is as set forth in SEQ ID NO: 39.

According to still further features in the described preferred embodiments the polypeptide is as set forth in SEQ ID NO: 37 or 38.

According to still a further aspect of the present invention there is provided an isolated polynucleotide as set forth in SEQ ID NO: 39.

According to still a further aspect of the present invention there is provided an isolated polypeptide as set forth in SEQ ID NO: 37 or 38.

According to still a further aspect of the present invention there is provided an isolated polypeptide comprising an amino acid sequence at least 70% identical to SEQ ID NO: 37, as determined using the LALIGN software of EMBnet Switzerland (http://www.ch.embnet.org/index.html) using default parameters or an active portion thereof.

According to still a further aspect of the present invention there is provided an antibody or an antibody fragment being capable of binding a polypeptide having an amino acid sequence at least 70% identical to SEQ ID NO: 37, as determined using the LALIGN software of EMBnet Switzerland (http://www.ch.embnet.org/index.html) using default parameters.

According to still a further aspect of the present invention there is provided an oligonucleotide hybridizable with a nucleic acid sequence encoding a polypeptide having an amino acid at least 70% identical to SEQ ID NO: 37, as determined using the LALIGN software of EMBnet Switzerland (http://www.ch.embnet.org/index.html) using default parameters.

According to still a further aspect of the present invention there is provided a pharmaceutical composition comprising a therapeutically effective amount of a polypeptide having an amino acid sequence at least 70% identical to SEQ ID NO: 37, as determined using the LALIGN software of EMBnet Switzerland (http://www.ch.embnet.org/index.html) using default parameters and a pharmaceutically acceptable carrier or diluent.

According to still a further aspect of the present invention there is provided a method of treating IL10-R-B-related disease in a subject, the method comprising upregulating in the subject expression of a polypeptide having an amino acid sequence at least 70% identical to SEQ ID NO: 37 as determined using the LALIGN software of EMBnet Switzerland (http://www.ch.embnet.org/index.html) using default parameters.

According to still a further aspect of the present invention there is provided an isolated polynucleotide comprising a nucleic acid sequence encoding a polypeptide having an amino acid sequence at least 80% identical to SEQ ID NO: 41, as determined using the LALIGN software of EMBnet Switzerland (http://www.ch.embnet.org/index.html) using default parameters.

According to still further features in the described preferred embodiments the nucleic acid sequence is as set forth in SEQ ID NO: 43 or 40.

According to still further features in the described preferred embodiments the polypeptide is as set forth in SEQ ID NO: 41 or 42.

According to still a further aspect of the present invention there is provided an isolated polynucleotide as set forth in SEQ ID NO: 43 or 40.

According to still a further aspect of the present invention there is provided an isolated polypeptide as set forth in SEQ ID NO: 41 or 42.

According to still a further aspect of the present invention there is provided an isolated polypeptide comprising an amino acid sequence at least 80% identical to SEQ ID NO: 41, as determined using the LALIGN software of EMBnet Switzerland (http://www.ch.embnet.org/index.html) using default parameters or an active portion thereof.

According to still a further aspect of the present invention there is provided an antibody or an antibody fragment being capable of binding a polypeptide having an amino acid sequence at least 80% identical to SEQ ID NO: 41, as determined using the LALIGN software of EMBnet Switzerland (http://www.ch.embnet.org/index.html) using default parameters.

According to still a further aspect of the present invention there is provided an oligonucleotide hybridizable with a nucleic acid sequence encoding a polypeptide having an amino acid at least 80% identical to SEQ ID NO: 41, as determined using the LALIGN software of EMBnet Switzerland (http://www.ch.embnet.org/index.html) using default parameters.

According to still a further aspect of the present invention there is provided a pharmaceutical composition comprising a therapeutically effective amount of a polypeptide having an amino acid sequence at least 80% identical to SEQ ID NO: 41, as determined using the LALIGN software of EMBnet Switzerland (http://www.ch.embnet.org/index.html) using default parameters and a pharmaceutically acceptable carrier or diluent.

According to still a further aspect of the present invention there is provided a method of treating INR1-related disease in a subject, the method comprising upregulating in the subject expression of a polypeptide having an amino acid sequence at least 80% identical to SEQ ID NO: 41 as determined using the LALIGN software of EMBnet Switzerland (http://www.ch.embnet.org/index.html) using default parameters.

According to still further features in the described preferred embodiments the upregulating expression of the polypeptide is effected by: (i) administering the polypeptide to the subject; and/or (ii) administering an expressible polynucleotide encoding the polypeptide.

The present invention successfully addresses the shortcomings of the presently known configurations by providing novel soluble polypeptides and polynucleotides encoding thereof, which can be used in the diagnosis and treatment of a wide range of diseases.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b present the nucleic acid sequence (FIG. 1a) and amino acid sequence (FIG. 1b) of the Met variant of the present invention (SEQ ID NO: 3 and 1, respectively).

FIG. 2 is a schematic illustration depicting a graphical viewer scheme presenting the new variant of Met (transcript_{—}9) as compred to the wild type mRNA. The ESTs supporting the new variant are indicated. Transcript indicated as “0” represents known mRNA. The color code is as follows: red=genomic DNA; pink=refseq mRNA; light blue=known genbank mRNAs; purple=ESTs aligned in the same directinality as their annotation; black=ESTs aligned in the opposite directinality to their annotation; grey=ESTs without direction annotation; dark blue=predicted transcripts; turquoise=predicted polypeptide.

FIG. 3 is an amino acid sequence alignment between wild-type c-Met protein and the protein variant of the present invention, as determined using the Smith and Waterman model query db, with the following parameters: −mode=qglobal −onestrand −gapext=0 −matrix=identity −out=g −gapop=40 −dfmt=fastap.

FIG. 4 is a schematic illustration showing the protein domain structure of wild-type c-Met protein and the variant of the present invention (SEQ ID NO: 1). Unique region is indicated by U (SEQ ID NO: 2).

FIGS. 5a-b present the nucleic acid sequence (FIG. 5a) and amino acid sequence (FIG. 5b) of the IL-6 variant of the present invention (SEQ ID NO: 7 and 5, respectively). Start and stop codons are highlighted. Unique sequence region is highlighted.

FIG. 6 is a schematic illustration depicting a graphical viewer scheme presenting the new variant of IL-6 (transcript_{—}6) as compred to the wild type mRNA. The ESTs supporting the new variant are indicated. Transcript indicated as “0” represents known mRNA. The color code is as follows: red=genomic DNA; pink=refseq mRNA; light blue=known genbank mRNAs; purple=ESTs aligned in the same directinality as their annotation; black=ESTs aligned in the opposite directinality to their annotation; grey=ESTs without direction annotation; dark blue=predicted transcripts; turquoise=predicted polypeptide.

FIG. 7 is an amino acid sequence alignment between wild-type IL-6 protein and the protein variant of the present invention, as determined using the Smith and Waterman model query db, with the following parameters: −mode=qglobal −onestrand −gapext=0 −matrix=identity −out=g −gapop=40 −dfmt=fastap.

FIG. 8 is a schematic illustration showing the protein domain structure of wild-type IL-6 protein and the variant of the present invention (SEQ ID NO: 5). Unique region is indicated by U (SEQ ID NO: 6).

FIGS. 9a-b present the nucleic acid sequence (FIG. 9a) and amino acid sequence (FIG. 9b) of the IL7 T7 variant of the present invention (SEQ ID NO: 11 and 9, respectively). Start and stop codons are highlighted. Unique sequence region is highlighted.

FIGS. 9c-d present the nucleic acid sequence (FIG. 9c) and amino acid sequence (FIG. 9d) of the IL7 T8 variant of the present invention (SEQ ID NO: 15 and 13, respectively). Start and stop codons are highlighted. Unique sequence region is highlighted.

FIG. 10 is a schematic illustration depicting a graphical viewer scheme presenting the new variants of IL7 (Transcript_{—}7 and Transcript 8) as compred to the wild type mRNA. The ESTs supporting the new variant are indicated. Transcript indicated as “0” represents known mRNA. The color code is as follows: red=genomic DNA; pink=refseq mRNA; light blue=known genbank mRNAs; purple=ESTs aligned in the same directinality as their annotation; black=ESTs aligned in the opposite directinality to their annotation; grey=ESTs without direction annotation; dark blue=predicted transcripts; turquoise=predicted polypeptide.

FIGS. 11a-b are amino acid sequence alignment between wild-type IL-7 protein and the protein variants (T7 and T8) of the present invention, as determined using the Smith and Waterman model query db, with the following parameters: −mode=qglobal −onestrand −gapext=0−matrix=identity −out=g −gapop=40 −dfmt=fastap.

FIG. 12 is a schematic illustration showing the protein domain structure of wild-type IL-7 protein and the variants of the present invention (SEQ ID NOs: 9 and 13). Unique regions are indicated by U (SEQ ID NO: 10 and 14).

FIGS. 13a-b present the nucleic acid sequence (FIG. 13a) and amino acid sequence (FIG. 13b) of the TNFR9 variant of the present invention (SEQ ID NO: 19 and 17, respectively). Start and stop codons are highlighted. Unique sequence region is highlighted.

FIG. 14 is a schematic illustration depicting a graphical viewer scheme presenting the new variant of TNFR9 (Transcript_{—}4) as compred to the wild type mRNA. The ESTs supporting the new variant are indicated. Transcript indicated as “0” represents known mRNA. The color code is as follows: red=genomic DNA; pink=refseq mRNA; light blue=known genbank mRNAs; purple=ESTs aligned in the same directinality as their annotation; black=ESTs aligned in the opposite directinality to their annotation; grey=ESTs without direction annotation; dark blue=predicted transcripts; turquoise=predicted polypeptide.

FIG. 15 is an amino acid sequence alignment between wild-type TNFR9 protein and the protein variant of the present invention, as determined using the Smith and Waterman model query db, with the following parameters: −mode=qglobal −onestrand −gapext=0 −matrix=identity −out=g −gapop=40 −dfmt=fastap.

FIG. 16 is a schematic illustration showing the protein domain structure of wild-type TNFR9 protein and the variant of the present invention (SEQ ID NO: 17). Unique region is indicated by U (SEQ ID NO: 18).

FIGS. 17a-b present the nucleic acid sequence (FIG. 17a) and amino acid sequence (FIG. 17b) of the IL-4R T4 variant of the present invention (SEQ ID NO: 23 and 21, respectively). Start and stop codons are highlighted. Unique sequence region is highlighted.

FIG. 17c present the nucleic acid sequence of the IL-4R T11 variant of the present invention (SEQ ID NO: 27). Start and stop codons are highlighted. Unique sequence region is highlighted.

FIG. 18 present the amino acid sequence of the IL-4R T11 variant of the present invention (SEQ ID NO: 25). Unique sequence region is highlighted.

FIGS. 19a-b are amino acid sequence alignments between wild-type IL-4R protein and the protein variants of the present invention (FIG. 19a-alignment of IL-4R-T4, and FIG. 19b alignment of IL-4R-T11), as determined using the Smith and Waterman model query db, with the following parameters: −mode=qglobal −onestrand −gapext=0 −matrix=identity −out=g −gapop=40 −dfmt=fastap.

FIG. 20 is a schematic illustration showing the protein domain structure of wild-type IL-4R protein and the variants of the present invention (SEQ ID NOs: 21 and 25). Unique regions are indicated by U (SEQ ID NOs: 22 and 26).

FIGS. 21a-b present the nucleic acid sequence (FIG. 21a) and amino acid sequence (FIG. 21b) of the TGR2 variant of the present invention (SEQ ID NO: 31 and 29, respectively). Start and stop codons are highlighted. Unique sequence region is highlighted.

FIG. 22 is a schematic illustration depicting a viewer scheme presenting the new variant of TGR2 (Transcript_{—}7) as compred to the wild type mRNA. The ESTs supporting the new variant are indicated. Transcript indicated as “0” represents known mRNA. The color code is as follows: red=genomic DNA; pink=refseq mRNA; light blue=known genbank mRNAs; purple=ESTs aligned in the same directinality as their annotation; black=ESTs aligned in the opposite directinality to their annotation; grey=ESTs without direction annotation; dark blue=predicted transcripts; turquoise=predicted polypeptide.

FIG. 23 is an amino acid sequence alignment between wild-type TGR2 protein and the protein variant of the present invention, as determined using the Smith and Waterman model query db, with the following parameters: −mode=qglobal −onestrand −gapext=0 −matrix=identity −out=g −gapop=40 −dfmt=fastap.

FIG. 24 is a schematic illustration showing the protein domain structure of wild-type TGR2 protein and the variant of the present invention (SEQ ID NO: 29). Unique region is indicated by U (SEQ ID NO: 30).

FIGS. 25a-b present the nucleic acid sequence (FIG. 25a) and amino acid sequence (FIG. 25b) of the ITAV variant of the present invention (SEQ ID NO: 35 and 33, respectively). Start and stop codons are highlighted. Unique sequence region is highlighted.

FIG. 26 is a schematic illustration depicting a viewer scheme presenting the new variant of ITAV (Transcipt_{—}3) as compred to the wild type mRNA. The ESTs supporting the new variant are indicated. Transcript indicated as “0” represents known mRNA. The color code is as follows: red=genomic DNA; pink=refseq mRNA; light blue=known genbank mRNAs; purple=ESTs aligned in the same directinality as their annotation; black=ESTs aligned in the opposite directinality to their annotation; grey=ESTs without direction annotation; dark blue=predicted transcripts; turquoise=predicted polypeptide.

FIG. 27 is an amino acid sequence alignment between wild-type ITAV protein and the protein variant of the present invention, as determined using the Smith and Waterman model query db, with the following parameters: −mode=qglobal −onestrand −gapext=0 −matrix=identity −out=g −gapop=40 −dfmt=fastap.

FIG. 28 is a schematic illustration showing the protein domain structure of wild-type ITAV protein and the variant of the present invention (SEQ ID NO: 33). Unique region is indicated by U (SEQ ID NO: 34).

FIGS. 29a-b present the nucleic acid sequence (FIG. 29a) and amino acid sequence (FIG. 29b) of the IL-10-R-β variant of the present invention (SEQ ID NO: 39 and 37, respectively). Start and stop codons are highlighted. Unique sequence region is highlighted.

FIG. 30 is a schematic illustration depicting the viewer scheme presenting the new variant of IL-10-R-β (Transcript_{—}1) as compred to the wild type mRNA. The ESTs supporting the new variant are indicated. Transcript indicated as “0” represents known mRNA. The color code is as follows: red=genomic DNA; pink=refseq mRNA; light blue=known genbank mRNAs; purple=ESTs aligned in the same directinality as their annotation; black=ESTs aligned in the opposite directinality to their annotation; grey=ESTs without direction annotation; dark blue=predicted transcripts; turquoise=predicted polypeptide.

FIG. 31 is an amino acid sequence alignment between wild-type IL-10-R-β protein and the protein variant of the present invention, as determined using the Smith and Waterman model query db, with the following parameters: −mode=qglobal −onestrand −gapext=0 −matrix=identity −out=g −gapop=40 −dfmt=fastap.

FIG. 32 is a schematic illustration showing the protein domain structure of wild-type IL-10-R-β protein and the variant of the present invention (SEQ ID NO: 37). Unique region is indicated by U (SEQ ID NO: 38).

FIGS. 33a-b present the nucleic acid sequence (FIG. 33a) and amino acid sequence (FIG. 33b) of the INR1 variant of the present invention (SEQ ID NO: 43 and 41, respectively). Start and stop codons are highlighted. Unique sequence region is highlighted.

FIG. 34 is a schematic illustration depicting a viewer scheme presenting the new variant of INR1 (Transcript_{—}11) as compred to the wild type mRNA. The ESTs supporting the new variant are indicated. Transcript indicated as “0” represents known mRNA. The color code is as follows: red=genomic DNA; pink=refseq mRNA; light blue=known genbank mRNAs; purple=ESTs aligned in the same directinality as their annotation; black=ESTs aligned in the opposite directinality to their annotation; grey=ESTs without direction annotation; dark blue=predicted transcripts; turquoise=predicted polypeptide.

FIG. 35 is an amino acid sequence alignment between wild-type INR1 protein and the protein variant of the present invention, as determined using the Smith and Waterman model query db, with the following parameters: −mode=qglobal −onestrand −gapext=0 −matrix=identity −out=g −gapop=40 −dfmt=fastap, FIG. 36 is a schematic illustration showing the protein domain structure of wild-type INR1 protein and the variant of the present invention (SEQ ID NO: 41). Unique region is indicated by U (SEQ ID NO: 42).

### DESCRIPTION OF THE PREFERRED EMBODIMENTS

The present invention is of novel soluble polypeptides and polynucleotides encoding same, which can be used for the diagnosis and treatment of a wide range of diseases, such as cancer and inflammatory diseases.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Extracellular proteins including receptors and their corresponding ligands play active roles in the formation, differentiation and maintenance of multicellular organisms. Any fate of an individual cell including proliferation, migration, differentiation, or interaction with other cells, is typically governed by information received from other cells and/or the immediate environment. This information is often transmitted by secreted polypeptides such as, mitogenic factors, survival factors, cytotoxic factors, differentiation factors, neuropeptides, and hormones, which are, in turn, received and interpreted by diverse cell receptors or membrane-bound proteins. These secreted polypeptides are normally processed by the cellular secretory pathway to reach their site of action in the extracellular environment.

Secreted proteins have various industrial applications, including as pharmaceuticals, diagnostics, biosensors and bioreactors. Most protein drugs available at present, such as thrombolytic polynucleotide or polypeptide sequences of this aspect of the present invention, interferons, interleukins, erythropoietins, colony stimulating factors, and various other cytokines, are secretory proteins. Their receptors, which are membrane proteins, also have potential as therapeutic or diagnostic polynucleotide or polypeptide sequences of this aspect of the present invention. For example, receptor immunoadhesins, for instance, can be employed as therapeutic polynucleotide or polypeptide sequences of this aspect of the present invention to block receptor-ligand interactions. The membrane-bound proteins can also be employed for screening of potential peptide or small molecule inhibitors of the relevant receptor/ligand interaction.

For these reasons, efforts are being undertaken by both industry and academia to identify novel, native, secreted proteins. Many efforts are focused on the screening of mammalian recombinant DNA libraries to identify the coding sequences for such proteins. Examples of such screening methods and techniques are described in, for example, Klein et al., Proc. Natl. Acad. Sci. 93:7108-7113 (1996); U.S. Pat. No. 5,536,637

The present inventors have previously designed algorithms which allow for the mass prediction of yet unknown gene products and for annotating same [see U.S. Pat. No. 6,625,545; U.S. patent application Ser. No. 10/426,002; a U.S. patent application entitled METHODS AND SYSTEMS FOR ANNOTATING BIOMOLECULAR SEQUENCES (Attorney Docket No. 26940), filed concurrently herewith, assigned to the same assignee hereof and contains subject matter related, in certain respects, to the subject matter of the instant application, the teachings of all of which are incorporated herein by reference; and Example 1 of the Examples section which follows].

While applying the above-mentioned algorithms, the present inventors uncovered novel naturally occurring variants of extracellular gene products, which, as is described in the Examples section which follows, play pivotal roles in disease onset and progression. As such these variants can be used to design therapeutic and diagnostic tools for a wide range of diseases.

Met Splice Variant

According to one aspect of the present invention there is provided an isolated polynucleotide comprising a nucleic acid sequence encoding at least an active portion of a polypeptide having an amino acid sequence, which is at least 50%, at least 55%, at least 60%, at least 65%, at least 70%, at least 75%, at least 80%, %, at least 85%, %, at least 90%, at least 95% or more, say 100% identical to SEQ ID NO: 1, as determined using the LALIGN software of EMBnet Switzerland (http://www.ch.embnet.org/index.html) using default parameters.

As used herein the phrase “an isolated polynucleotide” refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

According to one embodiment of this aspect of the present invention the nucleic acid sequence is as set forth in SEQ ID NO: 3 or 4.

Preferably, the polypeptide of this aspect of the present invention is at least an active portion of a naturally occurring protein product of a Met gene (Swissprot Locus No.: MET_HUMAN) and homologues thereof.

As used hereinabove the phrase “active portion” refers to an amino acid sequence portion which is capable of displaying one or more functions of the Met polypeptides of the present invention. Examples include but are not limited to ligand binding, antibody specific recognition, inhibition of cell-proliferation, scattering, angiogenesis, motility, morphogenesis and/or invasion (see Example 2 of the Examples section).

Thus, the polynucleotide according to this aspect of the present invention preferably encodes a polypeptide, which is as set forth in SEQ ID NO: 1 or 2.

The isolated polynucleotides of this aspect of the present invention can be qualified using a hybridization assay by incubating the isolated polynucleotides described above with a probe having the sequence set forth in SEQ ID NO: 3 or 4 under moderate to stringent hybridization conditions.

Moderate to stringent hybridization conditions are characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

The present invention also encompasses novel polypeptides (e.g., of the Met gene) or portions thereof, which are encoded by the isolated polynucleotide and respective nucleic acid fragments thereof described hereinabove.

Thus, the present invention also encompasses polypeptides encoded by the novel nucleic acids of the present invention. The amino acid sequences of these novel polypeptides are set forth in SEQ ID NO: 1 or 2. The present invention also encompasses homologues of these polypeptides, such homologues can be at least 50%, at least 55%, at least 60%, at least 65%, at least 70%, at least 73%, at least 77%, at least 80%, at least 85%, at least 95% or more say 100% identical to SEQ ID NO: 1 or 2. Finally, the present invention also encompasses fragments of the above described polypeptides and polypeptides having mutations, such as deletions, insertions or substitutions of one or more amino acids, either naturally occurring or man induced, either randomly or in a targeted fashion.

As is mentioned hereinabove and in Example 2 of the Examples section which follows, the biomolecular sequences of this aspect of the present invention may be used as valuable therapeutic tools in the treatment of “Met-related diseases”, since, without being bound by theory, they are devoid of a transmembrane and intracellular domains while retain the extracellular region of Met (i.e., HGF binding site) and therefore are likely to compete with HGF binding to the functional, membrane bound, Met receptor and as a consequence block Met activation and signaling pathway.

The above-mentioned “Met-related disease” refers to a disease in which Met-activity and/or expression contribute to disease onset and/or progression. Examples of Met-related diseases include, but are not limited to, cancer, such as, hereditary and sporadic papillary renal carcinoma, breast cancer, ovarian cancer, childhood hepatocellular carcinoma, metastatic head and neck squamous cell carcinomas, lung cancer (e.g., non-small cell lung cancer, small cell lung cancer), prostate cancer, pancreatic cancer and gastric cancer, and other diseases such as diabetic retinopathy.

It will be appreciated that the polypeptides of this aspect of the present invention may also have agonistic properties. These include increasing the stability of Met-ligand (e.g., HFG), protection from proteolysis and modification of the pharmacokinetic properties of the ligand (i.e., increasing the half-life of the ligand, while decreasing the clearance thereof). As such, the biomolecular sequences of this aspect of the present invention may be used to treat conditions or diseases in which Met plays a favorable role. Examples include, but are not limited to, regenerative processes such as wound healing and conditions, which require enhanced angiogenesis such as atherosclerotic diseases, ischemic conditions and diabetes. As mentioned the Met ligand is the hepatocyte growth factor, suggesting that the biomolecular sequences of this aspect of the present invention may have hepatoprotective properties and therefore may be used to diseases of the liver such as hepatic cirrhosis and hepatic dysfunction.

Thus, the present invention envisages treatment of the above-mentioned diseases by the provision of polynucleotide or polypeptide sequences of this aspect of the present invention, which are capable of upregulating expression of the polypeptides of the present invention in a subject in need thereof, as is further described hereinbelow. Such polynucleotide or polypeptide sequences of this aspect of the present invention and administration thereof are further described hereinbelow.

IL-6 Splice Variant

According to another aspect of the present invention there is provided an isolated polynucleotide comprising a nucleic acid sequence encoding at least an active portion of a polypeptide having an amino acid sequence, which is at least 50%, at least 55%, at least 60%, at least 65%, at least 70%, at least 75%, at least 80%, %, at least 85%, %, at least 90%, at least 95% or more, say 100% identical to SEQ ID NO: 5, as determined using the LALIGN software of EMBnet Switzerland (http://www.ch.embnet.org/index.html) using default parameters.

According to one embodiment of this aspect of the present invention the nucleic acid sequence is as set forth in SEQ ID NO: 7 or 8.

Preferably, the polypeptide of this aspect of the present invention is at least an active portion of a naturally occurring protein product of an IL-6 gene (Swissprot Locus No. IL6_HUMAN) and homologues thereof.

As used hereinabove the phrase “active portion” refers to an amino acid sequence portion which is capable of displaying one or more functions of the IL-6 polypeptides of the present invention. Examples include but are not limited antibody specific recognition and inhibition of IL-6 binding to the receptor (see Example 3 of the Examples section).

Thus, the polynucleotide according to this aspect of the present invention preferably encodes a polypeptide, which is as set forth in SEQ ID NO: 5 or 6.

The isolated polynucleotides of this aspect of the present invention can be qualified using a hybridization assay by incubating the isolated polynucleotides described above with a probe having the sequence set forth in SEQ ID NO: 7 or 8 under the above-described moderate to stringent hybridization conditions.

The present invention also encompasses novel polypeptides (e.g., products of the IL-6 gene) or portions thereof, which are encoded by the isolated polynucleotide and respective nucleic acid fragments thereof described hereinabove.

Thus, the present invention also encompasses polypeptides encoded by the novel nucleic acids of the present invention. The amino acid sequences of these novel polypeptides are set forth in SEQ ID NO: 5 or 6. The present invention also encompasses homologues of these polypeptides, such homologues can be at least 50%, at least 55%, at least 60%, at least 65%, at least 70%, at least 73%, at least 77%, at least 80%, at least 85%, at least 95% or more say 100% identical to SEQ ID NO: 5 or 6. Finally, the present invention also encompasses fragments of the above described polypeptides and polypeptides having mutations, such as deletions, insertions or substitutions of one or more amino acids, either naturally occurring or man induced, either randomly or in a targeted fashion.

As is mentioned hereinabove and in Example 3 of the Examples section which follows, the biomolecular sequences of this aspect of the present invention may be used as valuable therapeutic tools in the treatment of “IL-6-related diseases”, since, without being bound by theory, the IL-6 splice variant of this aspect of the present invention (SEQ ID NO: 5), contains the N-terminal 157 amino acids of wild-type IL-6, while lacks the last 50 amino acids of the protein, and as such may serve as an antagonist of IL-6 by several mechanisms. For example, this polypeptide variant can exhibit binding only to IL-6Rα, and no, or reduced binding to gp130, the second IL-6 receptor subunit. Since gp130 is the signaling subunit of the IL-6R complex, IL-6 splice variant of this aspect of the present invention, will not be able to activate the receptor. Thus it might serve as an antagonist of IL-6 signaling by binding the IL-6 receptor without activating it.

The above-mentioned “IL-6-related disease” refers to a disease in which IL-6-activity and/or expression contributes to disease onset and/or progression. Examples include, but are not limited to, inflammatory, autoimmune, and malignant diseases, such as, rheumatoid arthritis (RA), Castleman's disease, Crohn's disease, multiple myeloma/plasmacytoma, mesangial proliferative glomerulonephritis, psoriasis and Kaposi's sarcoma.

Thus, this aspect of the present invention envisages treatment of the above-mentioned diseases by the provision of polynucleotide or polypeptide sequences of this aspect of the present invention, which are capable of upregulating expression of the polypeptides of the present invention in a subject in need thereof, as is further described hereinbelow. Such polynucleotide or polypeptide sequences of this aspect of the present invention and administration thereof are further described hereinbelow.

IL-7 Splice Variants

According to yet another aspect of the present invention there is provided an isolated polynucleotide comprising a nucleic acid sequence encoding at least an active portion of a polypeptide having an amino acid sequence, which is at least 50%, at least 55%, at least 60%, at least 65%, at least 70%, at least 75%, at least 80%, %, at least 85%, %, at least 90%, at least 95% or more, say 100% identical to SEQ ID NO: 9 or 13, as determined using the LALIGN software of EMBnet Switzerland (http://www.ch.embnet.org/index.html) using default parameters.

According to one embodiment of this aspect of the present invention the nucleic acid sequence is as set forth in SEQ ID NO: 11, 12, 15 or 16.

Preferably, the polypeptide of this aspect of the present invention is at least an active portion of a naturally occurring protein product of an IL-7 gene (Swissprot Locus No. IL7_HUMAN) and homologues thereof.

As used hereinabove the phrase “active portion” refers to an amino acid sequence portion which is capable of displaying one or more functions of the IL-7 polypeptides of the present invention. Examples include but are not limited to, antibody specific recognition, inhibition of IL-7 binding to the receptor, enhancement of anti-tumor immunogenic reaction, reduction of tumor-induced suppression of immunogenic reaction, inhibition of cell proliferation (e.g., B-cells, see Example 4 of the Examples section).

Thus, the polynucleotide according to this aspect of the present invention preferably encodes a polypeptide, which is as set forth in SEQ ID NO: 9, 10, 13 or 14.

The isolated polynucleotides of this aspect of the present invention can be qualified using a hybridization assay by incubating the isolated polynucleotides described above with a probe having the sequence set forth in SEQ ID NO: 11, 12, 15 or 16 under the above-described moderate to stringent hybridization conditions.

The present invention also encompasses novel polypeptides (e.g., of the IL-7 gene) or portions thereof, which are encoded by the isolated polynucleotide and respective nucleic acid fragments thereof described hereinabove.

Thus, the present invention also encompasses polypeptides encoded by the novel nucleic acids of the present invention. The amino acid sequences of these novel polypeptides are set forth in SEQ ID NO: 9, 10, 13 or 14. The present invention also encompasses homologues of these polypeptides, such homologues can be at least 50%, at least 55%, at least 60%, at least 65%, at least 70%, at least 73%, at least 77%, at least 80%, at least 85%, at least 95% or more say 100% identical to SEQ ID NO: 9, 10, 13 or 14. Finally, the present invention also encompasses fragments of the above described polypeptides and polypeptides having mutations, such as deletions, insertions or substitutions of one or more amino acids, either naturally occurring or man induced, either randomly or in a targeted fashion.

As is mentioned hereinabove and in Example 4 of the Examples section which follows, the biomolecular sequences of this aspect of the present invention may be used as valuable therapeutic tools in the treatment of “IL-7-related diseases”, since, without being bound by theory, they may bing only one IL-7 receptor subuit to thereby produce dysfunctional ligand-receptor complexes.

The above-mentioned “IL-7-related disease” refers to a disease in which IL-7-activity and/or expression contributes to disease onset and/or progression. Examples of IL-7-related diseases include, but are not limited to cancer, such as acute and chronic lymphocytic leukemia, acute myelogenous leukemia, Sezary's syndome, Burkitt's lymphoma and Hodgkin's disease.

It will be appreciated that the polypeptides of this aspect of the present invention may also have agonistic properties, such as by binding to the IL-7 receptor with enhanced affinity as compared to the wild-type IL-7. As such, the biomolecular sequences of this aspect of the present invention may be used to treat conditions or diseases in which IL-7 plays a favorable role. Examples include, but are not limited to, cancer, such as melanoma, renal and colorectal cancer, in which IL-7 plays a therapeutic role by eliciting anti-tumor immunogenic responses. Furthermore, it is well established that IL-7 controls the growth and proliferation of immature B-cells and can stimulate the development of bone marrow cells into T-cells and B-cell precursors. Thus the polypeptides of this aspect of the present invention may be used establish a spectrum of lymphoid cell types following radiotherapy or chemotherapy.

Thus, this aspect of the present invention envisages treatment of the above-mentioned diseases by the provision of polynucleotide or polypeptide sequences of this aspect of the present invention, which are capable of upregulating expression of the polypeptides of the present invention in a subject in need thereof, as is further described hereinbelow. Such polynucleotide or polypeptide sequences of this aspect of the present invention and administration thereof are further described hereinbelow.

Tumor Necrosis Factor Receptor 9 (TNR-9)/4-1BBR Splice Variant

According to still another aspect of the present invention there is provided an isolated polynucleotide comprising a nucleic acid sequence encoding at least an active portion of a polypeptide having an amino acid sequence, which is at least 50%, at least 55%, at least 60%, at least 65%, at least 70%, at least 75%, at least 80%, %, at least 85%, %, at least 90%, at least 95% or more, say 100% identical to SEQ ID NO: 17, as determined using the LALIGN software of EMBnet Switzerland (http://www.ch.embnet.org/index.html) using default parameters.

According to one embodiment of this aspect of the present invention the nucleic acid sequence is as set forth in SEQ ID NO: 19 or 20.

Preferably, the polypeptide of this aspect of the present invention is at least an active portion of a naturally occurring protein product of a TNR9 gene (Swissprot Locus No. TNR9_HUMAN) and homologues thereof.

As used hereinabove the phrase “active portion” refers to an amino acid sequence portion which is capable of displaying one or more functions of the TNFR9 polypeptides of the present invention. Examples include but are not limited to 4-1BB binding, antibody specific recognition, inhibition of IL-2 production, cell-proliferation and differentiation, clonal expansion and survival of CD38+ cells, signaling (see Example 5 of the Examples section).

Thus, the polynucleotide according to this aspect of the present invention preferably encodes a polypeptide, which is as set forth in SEQ ID NO: 17 or 18.

The isolated polynucleotides of this aspect of the present invention can be qualified using a hybridization assay by incubating the isolated polynucleotides described above with a probe having the sequence set forth in SEQ ID NO: 19 or 20 under the above-described moderate to stringent hybridization conditions.

The present invention also encompasses novel polypeptides (e.g., of the TNR9 gene) or portions thereof, which are encoded by the isolated polynucleotide and respective nucleic acid fragments thereof described hereinabove.

Thus, the present invention also encompasses polypeptides encoded by the novel nucleic acids of the present invention. The amino acid sequences of these novel polypeptides are set forth in SEQ ID NO: 17 or 18. The present invention also encompasses homologues of these polypeptides, such homologues can be at least 50%, at least 55%, at least 60%, at least 65%, at least 70%, at least 73%, at least 77%, at least 80%, at least 85%, at least 95% or more say 100% identical to SEQ ID NO: 17 or 18. Finally, the present invention also encompasses fragments of the above described polypeptides and polypeptides having mutations, such as deletions, insertions or substitutions of one or more amino acids, either naturally occurring or man induced, either randomly or in a targeted fashion.

As is mentioned hereinabove and in Example 5 of the Examples section which follows, the biomolecular sequences of this aspect of the present invention may be used as valuable therapeutic tools in the treatment of “TNR9-related diseases”, since, without being bound by theory, they are devoid of a transmembrane and intracellular domains while retain the extracellular region of TNR9 and therefore are likely to compete with 4-1BBL binding to the functional, membrane bound, TNR9 receptor and as a consequence block TNR9 activation and signaling pathway.

The above-mentioned “TNR9-related disease” refers to a disease in which TNR9-activity and/or expression contribute to disease onset and/or progression. Examples of TNR9-related diseases include, but are not limited to, myocardial inflammation, induced by coxackievirus B3, herpetic stromal keratitis (HSK) induced by HSV-1 and inflammatory diseases, such as multiple sclerosis and Crohn's disease as well as prevention of graft rejection and graft-versus-host disease.

It will be appreciated that the polypeptides of this aspect of the present invention may also have agonistic properties. These include increasing the stability of 4-1BB-ligand, protection from proteolysis and modification of the pharmacokinetic properties of the ligand (i.e., increasing the half-life of the ligand, while decreasing the clearance thereof). As such, the biomolecular sequences of this aspect of the present invention may be used to treat conditions or diseases in which TNR9 plays a favorable role. Examples include, but are not limited to, cancer, viral infections and autoimuune diseases such as spontaneous systemic lupos erythematosus (SLE), RA, and ulceratice colitis.

Thus, this aspect of the present invention envisages treatment of the above-mentioned diseases by the provision of polynucleotide or polypeptide sequences of this aspect of the present invention, which are capable of upregulating expression of the polypeptides of the present invention in a subject in need thereof, as is further described hereinbelow. Such polynucleotide or polypeptide sequences of this aspect of the present invention and administration thereof are further described hereinbelow.

Interleukin 4 Receptor (IL4R) Splice Variants

According to an additional aspect of the present invention there is provided an isolated polynucleotide comprising a nucleic acid sequence encoding at least an active portion of a polypeptide having an amino acid sequence, which is at least 50%, at least 55%, at least 60%, at least 65%, at least 70%, at least 75%, at least 80%, %, at least 85%, %, at least 90%, at least 95% or more, say 100% identical to SEQ ID NO: 21 or 25, as determined using the LALIGN software of EMBnet switzerland (http://www.ch.embnet.org/index.html) using default parameters.

According to one embodiment of this aspect of the present invention the nucleic acid sequence is as set forth in SEQ ID NO: 23, 24, 27 or 28.

Preferably, the polypeptide of this aspect of the present invention is at least an active portion of a naturally occurring protein product of an IL-4 receptor gene (Swissprot Locus No. IL4R_HUMAN) and homologues thereof.

As used hereinabove the phrase “active portion” refers to an amino acid sequence portion which is capable of displaying one or more functions of the IL-4R polypeptides of the present invention. Examples include but are not limited to ligand binding, antibody specific recognition and inhibition of IL-4 signaling (see Example 6 of the Examples section).

Thus, the polynucleotide according to this aspect of the present invention preferably encodes a polypeptide, which is as set forth in SEQ ID NO: 21, 22, 25 or 26.

The isolated polynucleotides of this aspect of the present invention can be qualified using a hybridization assay by incubating the isolated polynucleotides described above with a probe having the sequence set forth in SEQ ID NO: 23, 24, 27 or 28 under the above-described moderate to stringent hybridization conditions.

The present invention also encompasses novel polypeptides (e.g., of the IL-4R gene) or portions thereof, which are encoded by the isolated polynucleotide and respective nucleic acid fragments thereof described hereinabove.

Thus, the present invention also encompasses polypeptides encoded by the novel nucleic acids of the present invention. The amino acid sequences of these novel polypeptides are set forth in SEQ ID NO: 21, 22, 25 or 26. The present invention also encompasses homologues of these polypeptides, such homologues can be at least 50%, at least 55%, at least 60%, at least 65%, at least 70%, at least 73%, at least 77%, at least 80%, at least 85%, at least 95% or more say 100% identical to SEQ ID NO: 21, 22, 25 or 26. Finally, the present invention also encompasses fragments of the above described polypeptides and polypeptides having mutations, such as deletions, insertions or substitutions of one or more amino acids, either naturally occurring or man induced, either randomly or in a targeted fashion.

As is mentioned hereinabove and in Example 6 of the Examples section which follows, the biomolecular sequences of this aspect of the present invention may be used as valuable therapeutic tools in the treatment of “IL-4R-related diseases”, since, without being bound by theory, they are devoid of a transmembrane and intracellular domains, while still retain a complete CRIA domain of the extracellular region of IL-4R and therefore are likely to compete with IL-4 binding to the functional, membrane bound, IL-4 receptor and as a consequence block IL-4 activation and signaling pathway.

The above-mentioned “IL-4R-related disease” refers to a disease in which IL-4R-activity and/or expression contribute to disease onset and/or progression. Examples of IL-4R-related diseases include, but are not limited to, asthma and allergic disorders, autoimmune diseases, such as lupus, transplant rejection and graft-versus-host diseases It will be appreciated that the polypeptides of this aspect of the present invention may also have agonistic properties. These include increasing the stability of IL-4R-ligand (e.g., IL-4), protection from proteolysis and modification of the pharmacokinetic properties of the ligand (i.e., increasing the half-life of the ligand, while decreasing the clearance thereof). As such, the biomolecular sequences of this aspect of the present invention may be used to treat conditions or diseases in which IL-4 plays a favorable role. Examples include, but are not limited to, cancer, such as, leukaemia, kaposi's sarcoma, lymphoma and non-small cell lung cancer, anaemia and rheumatoid arthritis.

Thus, this aspect of the present invention envisages treatment of the above-mentioned diseases by the provision of polynucleotide or polypeptide sequences of this aspect of the present invention, which are capable of upregulating expression of the polypeptides of the present invention in a subject in need thereof, as is further described hereinbelow. Such polynucleotide or polypeptide sequences of this aspect of the present invention and administration thereof are further described hereinbelow.

Transforming Growth Factor β Receptor Type II (TGF-β-R/TGR2) Splice Variant

According to yet an additional aspect of the present invention there is provided an isolated polynucleotide comprising a nucleic acid sequence encoding at least an active portion of a polypeptide having an amino acid sequence, which is at least 50%, at least 55%, at least 60%, at least 65%, at least 70%, at least 75%, at least 80%, %, at least 85%, %, at least 90%, at least 95% or more, say 100% identical to SEQ ID NO: 29, as determined using the LALIGN software of EMBnet Switzerland (http://www.ch.embnet.org/index.html) using default parameters.

According to one embodiment of this aspect of the present invention the nucleic acid sequence is as set forth in SEQ ID NO: 31 or 32.

Preferably, the polypeptide of this aspect of the present invention is at least an active portion of a naturally occurring protein product of a TGR2 gene (Swissprot Locus No. TGR2_HUMAN) and homologues thereof.

As used hereinabove the phrase “active portion” refers to an amino acid sequence portion which is capable of displaying one or more functions of the TGR2 polypeptides of the present invention. Examples include but are not limited to ligand binding, antibody specific recognition, inhibition of TGR2 signaling such as through the type I receptor and SMAD proteins or through P13K and p70S6K, tumor suppression and tumor promotion (see Example 7 of the Examples section).

Thus, the polynucleotide according to this aspect of the present invention preferably encodes a polypeptide, which is as set forth in SEQ ID NO: 29 or 30.

The isolated polynucleotides of this aspect of the present invention can be qualified using a hybridization assay by incubating the isolated polynucleotides described above with a probe having the sequence set forth in SEQ ID NO: 31 or 32 under the above-described moderate to stringent hybridization conditions.

The present invention also encompasses novel polypeptides (e.g., of the TGR2 gene) or portions thereof, which are encoded by the isolated polynucleotide and respective nucleic acid fragments thereof described hereinabove.

Thus, the present invention also encompasses polypeptides encoded by the novel nucleic acids of the present invention. The amino acid sequences of these novel polypeptides are set forth in SEQ ID NO: 29 or 30. The present invention also encompasses homologues of these polypeptides, such homologues can be at least 50%, at least 55%, at least 60%, at least 65%, at least 70%, at least 73%, at least 77%, at least 80%, at least 85%, at least 95% or more say 100% identical to SEQ ID NO: 29 or 30. Finally, the present invention also encompasses fragments of the above described polypeptides and polypeptides having mutations, such as deletions, insertions or substitutions of one or more amino acids, either naturally occurring or man induced, either randomly or in a targeted fashion.

As is mentioned hereinabove and in Example 7 of the Examples section which follows, the biomolecular sequences of this aspect of the present invention may be used as valuable therapeutic tools in the treatment of “TGR2-related diseases”, since, without being bound by theory, they are devoid of a transmembrane and intracellular domains while retain the extracellular region of TGR2 and therefore are likely to compete with TGF-β binding to the functional, membrane bound, TGR2 receptor and as a consequence block TGR2 activation and signaling pathway.

The above-mentioned “TGR2-related disease” refers to a disease in which TGR2-activity and/or expression contribute to disease onset and/or progression. Examples of TGR2-related diseases include, but are not limited to, cancer, such as glioblastoma where TGR2 acts as a tumor promoter (see Example 6 of the Examples section), organ remodeling diseases and fibrotic diseases, such as chronic renal disease or pulmonary fibrosis, scleroderma and eye scarring following glaucoma surgery.

It will be appreciated that the polypeptides of this aspect of the present invention may also have agonistic properties. These include increasing the stability of TGF β, protection from proteolysis and modification of the pharmacokinetic properties of the ligand (i.e., increasing the half-life of the ligand, while decreasing the clearance thereof). As such, the biomolecular sequences of this aspect of the present invention may be used to treat conditions or diseases in which TGR2 plays a favorable role, such as in cancer onset where TGR2 plays a protective role, atherosclerosis and other injury-induced hyperplasias such as restenosis, rhematoid arthritis, psoriasis, multiple sclerosis, osteoporesis and articular cartilage damage, in which tissue repair is achieved by promoting TGFb2 Activity. The polypeptides of this aspect of the present invention may also be used for wound healing.

Thus, this aspect of the present invention envisages treatment of the above-mentioned diseases by the provision of polynucleotide or polypeptide sequences of this aspect of the present invention, which are capable of upregulating expression of the polypeptides of the present invention in a subject in need thereof, as is further described hereinbelow.

Integrin-α-V (ITAV) Splice Variants

According to still an additional aspect of the present invention there is provided an isolated polynucleotide comprising a nucleic acid sequence encoding at least an active portion of a polypeptide having an amino acid sequence, which is at least 50%, at least 55%, at least 60%, at least 65%, at least 70%, at least 75%, at least 80%, %, at least 85%, %, at least 90%, at least 95% or more, say 100% identical to SEQ ID NO: 33, as determined using the LALIGN software of EMBnet Switzerland (http://www.ch.embnet.org/index.html) using default parameters.

According to one embodiment of this aspect of the present invention the nucleic acid sequence is as set forth in SEQ ID NO: 33 or 34.

Preferably, the polypeptide of this aspect of the present invention is at least an active portion of a naturally occurring protein product of a ITAV gene (Swissprot Locus No. ITAV_HUMAN) and homologues thereof.

As used hereinabove the phrase “active portion” refers to an amino acid sequence portion which is capable of displaying one or more functions of the ITAV polypeptides of the present invention. Examples include but are not limited to ligand (e.g., extracellular matrix proteins, fibronectin) or receptor-partner binding, antibody specific recognition, inhibition of cell survival, proliferation and migration (see Example 8 of the Examples section).

Thus, the polynucleotide according to this aspect of the present invention preferably encodes a polypeptide, which is as set forth in SEQ ID NO: 33 or 34.

The isolated polynucleotides of this aspect of the present invention can be qualified using a hybridization assay by incubating the isolated polynucleotides described above with a probe having the sequence set forth in SEQ ID NO: 35 or 36 under the above-described moderate to stringent hybridization conditions.

The present invention also encompasses novel polypeptides (e.g., of the ITAV gene) or portions thereof, which are encoded by the isolated polynucleotide and respective nucleic acid fragments thereof described hereinabove.

Thus, the present invention also encompasses polypeptides encoded by the novel nucleic acids of the present invention. The amino acid sequences of these novel polypeptides are set forth in SEQ ID NO: 33 or 34. The present invention also encompasses homologues of these polypeptides, such homologues can be at least 50%, at least 55%, at least 60%, at least 65%, at least 70%, at least 73%, at least 77%, at least 80%, at least 85%, at least 95% or more say 100% identical to SEQ ID NO: 33 or 34. Finally, the present invention also encompasses fragments of the above described polypeptides and polypeptides having mutations, such as deletions, insertions or substitutions of one or more amino acids, either naturally occurring or man induced, either randomly or in a targeted fashion.

As is mentioned hereinabove and in Example 8 of the Examples section which follows, the biomolecular sequences of this aspect of the present invention may be used as valuable therapeutic tools in the treatment of “ITAV-related diseases”, since, without being bound by theory, they are devoid of a transmembrane and intracellular domains while retain the extracellular region of ITAV and therefore are likely to compete with ligand binding to the functional, membrane bound, ITAV receptor and as a consequence block ITAV activation and signaling pathway. Alternatively these polypeptides can dimerize with a second receptor subunit to produce a dysfunctional heterodimeric complexes thereby blocking signaling.

The above-mentioned “ITAV-related disease” refers to a disease in which ITAV-activity and/or expression contributes to disease onset and/or progression. Examples of ITAV-related diseases include, but are not limited to, ocular diseases(e.g., persistent corneal epithelial defect), cancer (e.g., breast cancer, renal cancer, cervical cancer, colon cancer, prostate cancer, bladder cancer, lung cancer and melanoma), cardiovascular diseases (e.g., stroke and heart failure, atherosclerosis, restenosis, ischemia and reperfusion injury), immunological related diseases (e.g., immunodeficiency, allergies, asthma, psoriasis, RA and inflammatory bowl diseases e.g., Chrone's disease), metabolism related diseases, such as diabetes and diabetes related retinopathy, osteoporosis, sepsis and wound healing.

Thus, this aspect of the present invention envisages treatment of the above-mentioned diseases by the provision of agents, which are capable of upregulating expression of the polypeptides of the present invention in a subject in need thereof. Such agents and administration thereof are further described hereinbelow.

Interleukin-10 Receptor β Chain (IL-10-Rβ) Splice Variant

According to a further aspect of the present invention there is provided an isolated polynucleotide comprising a nucleic acid sequence encoding at least an active portion of a polypeptide having an amino acid sequence, which is at least 50%, at least 55%, at least 60%, at least 65%, at least 70%, at least 75%, at least 80%, %, at least 85%, %, at least 90%, at least 95% or more, say 100% identical to SEQ ID NO: 37, as determined using the LALIGN software of EMBnet Switzerland (http://www.ch.embnet.org/index.html) using default parameters.

According to one embodiment of this aspect of the present invention the nucleic acid sequence is as set forth in SEQ ID NO: 39.

Preferably, the polypeptide of this aspect of the present invention is at least an active portion of a naturally occurring protein product of an IL-10-Rβ gene (Swissprot Locus No. I10S_HUMAN) and homologues thereof.

As used hereinabove the phrase “active portion” refers to an amino acid sequence portion which is capable of displaying one or more functions of the IL-10-Rβ polypeptides of the present invention. Examples include, but are not limited to, ligand binding, antibody specific recognition, regulation of IL-10R signaling (e.g., STAT activation) and regulation of immune responses (see Example 9 of the Examples section).

Thus, the polynucleotide according to this aspect of the present invention preferably encodes a polypeptide, which is as set forth in SEQ ID NO: 37 or 38.

The isolated polynucleotides of this aspect of the present invention can be qualified using a hybridization assay by incubating the isolated polynucleotides described above with a probe having the sequence set forth in SEQ ID NO: 39 under the above-described moderate to stringent hybridization conditions.

The present invention also encompasses novel polypeptides (e.g., of the IL-10-Rβ gene) or portions thereof, which are encoded by the isolated polynucleotide and respective nucleic acid fragments thereof described hereinabove.

Thus, the present invention also encompasses polypeptides encoded by the novel nucleic acids of the present invention. The amino acid sequences of these novel polypeptides are set forth in SEQ ID NO: 37 or 38. The present invention also encompasses homologues of these polypeptides, such homologues can be at least 50%, at least 55%, at least 60%, at least 65%, at least 70%, at least 73%, at least 77%, at least 80%, at least 85%, at least 95% or more say 100% identical to SEQ ID NO: 37 or 38. Finally, the present invention also encompasses fragments of the above described polypeptides and polypeptides having mutations, such as deletions, insertions or substitutions of one or more amino acids, either naturally occurring or man induced, either randomly or in a targeted fashion.

As is mentioned hereinabove and in Example 9 of the Examples section which follows, the biomolecular sequences of this aspect of the present invention may be used as valuable therapeutic tools in the treatment of “IL-10-Rβ-related diseases”, due to their enhanced agonistic properties. These include increasing the stability of the ligand (e.g., IL-10), protection from proteolysis and modification of the pharmacokinetic properties of the ligand (i.e., increasing the half-life of the ligand, while decreasing the clearance thereof) to thereby increase the biological effects of the IL-10 signaling cascade. As such, the biomolecular sequences of this aspect of the present invention may be used to treat conditions or diseases in which IL-10R signaling plays a favorable role. Examples include, but are not limited to, inflammatory diseases, such as, psoriasis, inflammatory bowel diseases, Crohn's disease, colitis ulcerative, multiple sclerosis, RA, transplant rejection, allergic contact dermatitis, hepatitis C infection; HIV infection and atherosclerosis.

It will be appreciated, however, that since the polypeptide seuqneces of this aspect of the present invention are devoid of a transmembrane and intracellular domains while retain the extracellular region of IL-10-Rβ (i.e., IL-10 binding site), they are most likely to compete with IL-10 binding to the functional, membrane bound, IL-10-Rβ receptor and as a consequence block IL-10R activation and signaling pathway. Thus, due to their predicted antagonistic effects, the biomolecular sequences of this aspect of the present invention may be used to treat diseases which depend on IL-10R (i.e., activity and/or expression) for their onset or progression. Examples include, but are not limited to, cancer, such as lymphoma, melanoma and carcinoma, and infection with visceral leishmaniasis.

Thus, this aspect of the present invention envisages treatment of the above-mentioned diseases by the provision of agents, which are capable of upregulating expression of the polypeptides of the present invention in a subject in need thereof. Such agents and administration thereof are further described hereinbelow.

Interferon-α/β-receptor-1-INR1 Splice Variant

According to yet a further aspect of the present invention there is provided an isolated polynucleotide comprising a nucleic acid sequence encoding at least an active portion of a polypeptide having an amino acid sequence, which is at least 50%, at least 55%, at least 60%, at least 65%, at least 70%, at least 75%, at least 80%, %, at least 85%, %, at least 90%, at least 95% or more, say 100% identical to SEQ ID NO: 41, as determined using the LALIGN software of EMBnet Switzerland (http://www.ch.embnet.org/index.html) using default parameters.

According to one embodiment of this aspect of the present invention the nucleic acid sequence is as set forth in SEQ ID NO: 43 or 40.

Preferably, the polypeptide of this aspect of the present invention is at least an active portion of a naturally occurring protein product of an INR1 gene (Swissprot Locus No. INR1_HUMAN) and homologues thereof.

As used hereinabove the phrase “active portion” refers to an amino acid sequence portion which is capable of displaying one or more functions of the INR1 polypeptides of the present invention. Examples include but are not limited to ligand binding, antibody specific recognition, modulation of immune responses (see Example 10 of the Examples section).

Thus, the polynucleotide according to this aspect of the present invention preferably encodes a polypeptide, which is as set forth in SEQ ID NO: 41 or 42.

The isolated polynucleotides of this aspect of the present invention can be qualified using a hybridization assay by incubating the isolated polynucleotides described above with a probe having the sequence set forth in SEQ ID NO: 43 or 40 under the above-described moderate to stringent hybridization conditions.

The present invention also encompasses novel polypeptides (e.g., of the INR1 gene) or portions thereof, which are encoded by the isolated polynucleotide and respective nucleic acid fragments thereof described hereinabove.

Thus, the present invention also encompasses polypeptides encoded by the novel nucleic acids of the present invention. The amino acid sequences of these novel polypeptides are set forth in SEQ ID NO: 41 or 42. The present invention also encompasses homologues of these polypeptides, such homologues can be at least 50%, at least 55%, at least 60%, at least 65%, at least 70%, at least 73%, at least 77%, at least 80%, at least 85%, at least 95% or more say 100% identical to SEQ ID NO: 41 or 42. Finally, the present invention also encompasses fragments of the above described polypeptides and polypeptides having mutations, such as deletions, insertions or substitutions of one or more amino acids, either naturally occurring or man induced, either randomly or in a targeted fashion.

As is mentioned hereinabove and in Example 10 of the Examples section which follows, the biomolecular sequences of this aspect of the present invention may be used as valuable therapeutic tools in the treatment of “INR1-related diseases”, due to their enhanced agonistic properties. These include increasing the stability of the ligand (e.g., interferon), protection from proteolysis and modification of the pharmacokinetic properties of the ligand (i.e., increasing the half-life of the ligand, while decreasing the clearance thereof).

Thus, the biomolecular sequences may be used to treat a number of diseases in which INR1 plays a favorable role. Examples of such diseases include, but are not limited to, cancer, such as, solid tumors (e.g., glioblastoma, renal cell carcinoma, melanoma) and hematological malignancies [e.g., chronic myelogenous leukemia (CML), multiple myeloma, non-Hodgkin's lymphoma and hairy cell leukemia], viral infections (e.g., hepatitis B/C, herpes and human papilloma virus) and autoimmune diseases such as multiple sclerosis.

As mentioned hereinabove, the polypeptide sequences of the present invention can be used in a number of therapeutic applications. In such applications it is highly desirable to employ the minimal and most efficacious peptide regions, which still exert inhibitory function. Identification of such peptide regions can be effected using various approaches, including, for example, display techniques.

Thus, according to still a further aspect of the present invention there is provided a display library comprising a plurality of display vehicles (such as phages, viruses or bacteria) each displaying at least 6, at least 7, at least 8, at least 9, at least 10, 10-15, 12-17, 15-20, 15-30 or 20-50 consecutive amino acids derived from the polypeptide sequences of the present invention.

Methods of constructing such display libraries are well known in the art. Such methods are described in, for example, Young AC, et al., “The three-dimensional structures of a polysaccharide binding antibody to Cryptococcus neoformans and its complex with a peptide from a phage display library: implications for the identification of peptide mimotopes” J Mol Biol 1997 Dec. 12;274(4):622-34; Giebel LB et al. “Screening of cyclic peptide phage libraries identifies ligands that bind streptavidin with high affinities” Biochemistry 1995 Nov. 28;34(47):15430-5; Davies EL et al., “Selection of specific phage-display antibodies using libraries derived from chicken immunoglobulin genes” J Immunol Methods 1995 Oct. 12;186(1):125-35; Jones C RT al. “Current trends in molecular recognition and bioseparation” J Chromatogr A 1995 Jul. 14;707(1):3-22; Deng SJ et al. “Basis for selection of improved carbohydrate-binding single-chain antibodies from synthetic gene libraries” Proc Natl Acad Sci USA 1995 May 23;92(11):4992-6; and Deng SJ et al. “Selection of antibody single-chain variable fragments with improved carbohydrate binding by phage display” J Biol Chem 1994 Apr. 1;269(13):9533-8, which are incorporated herein by reference.

Peptide sequences which exhibit high therapeutic activity, such as by competing with wild type signaling proteins of the same signaling pathway, can be also uncovered using computational biology. Software programs useful for displaying three-dimensional structural models, such as RIBBONS (Carson, M., 1997. Methods in Enzymology 277, 25), 0 (Jones, TA. et al., 1991. Acta Crystallogr. A47, 110), DINO (DINO: Visualizing Structural Biology (2001) http://www.dino3d.org); and QUANTA, INSIGHT, SYBYL, MACROMODE, ICM, MOLMOL, RASMOL and GRASP (reviewed in Kraulis, J., 1991. Appl Crystallogr. 24, 946) can be utilized to model interactions between the polypeptides of the present invention and prospective peptide sequences to thereby identify peptides which display the highest probability of binding for example to a respective ligand (e.g., IL-10). Computational modeling of protein-peptide interactions has been successfully used in rational drug design, for further detail, see Lam et al., 1994. Science 263, 380; Wlodawer et al., 1993. Ann Rev Biochem. 62, 543; Appelt, 1993. Perspectives in Drug Discovery and Design 1, 23; Erickson, 1993. Perspectives in Drug Discovery and Design 1, 109, and Mauro MJ. et al., 2002. J Clin Oncol. 20, 325-34.

It will be appreciated that peptides identified according to the teachings of the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2—NH, CH2—S, CH2—S═O, O═C—NH, CH2—O, CH2—CH2, S═C—NH, CH═CH or CF═CH, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)—CO—), ester bonds (—C(R)H—C—O—O—C(R)—N—), ketomethylen bonds (—CO—CH2—), α-aza bonds (—NH—N(R)—CO—), wherein R is any alkyl, e.g., methyl, carba bonds (—CH2—NH—), hydroxyethylene bonds (—CH(OH)—CH2—), thioamide bonds (—CS—NH—), olefinic double bonds (—CH═CH—), retro amide bonds (—NH—CO—), peptide derivatives (—N(R)—CH2—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1) and non-conventional or modified amino acids (Table 2) which can be used with the present invention.

TABLE 1

| | Three-Letter | One-letter |
|---|---|---|
| Amino Acid | Abbreviation | Symbol |
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutyric acid | Abu | L-N-methylalanine | Nmala |
| α-amino- | Mgabu | L-N-methylarginine | Nmarg |
| α-methylbutyrate | | | |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1- | Nmbc | | |
| (2,2-diphenyl ethylamino) | | | |
| cyclopropane | | | |

Since the peptides of the present invention are preferably utilized in therapeutics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclicization does not severely interfere with peptide characteristics, cyclic forms of the peptide can also be utilized.

The peptides of present invention can be biochemically synthesized such as by using standard solid phase techniques. These methods include exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

Briefly, polynucleotides encoding the polypeptides of the present invention are first cloned into an appropriate nucleic acid construct (i.e., vector).

To enable cellular expression of the proteins of the present invention, the nucleic acid construct of the present invention further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any available promoter can be used by the present methodology dependent on the host cell (e.g., eukaryotic, prokaryotic).

The nucleic acid construct of the present invention can further include an enhancer, which can be adjacent or distant to the promoter sequence and can function in up regulating the transcription therefrom.

The constructs of the present methodology preferably further include an appropriate selectable marker and/or an origin of replication. Preferably, the construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Other then containing the necessary elements for the transcription and translation of the inserted coding sequence, the expression construct of the present invention can also include sequences engineered to enhance stability, production, purification, yield or toxicity of the expressed peptide. For example, the expression of a fusion protein or a cleavable fusion protein comprising Met variant of the present invention and a heterologous protein can be engineered. Such a fusion protein can be designed so that the fusion protein can be readily isolated by affinity chromatography; e.g., by immobilization on a column specific for the heterologous protein. Where a cleavage site is engineered between the Met moiety and the heterologous protein, the Met moiety can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

As mentioned hereinabove, a variety of prokaryotic or eukaryotic cells can be used as host-expression systems to express the polypeptides of the present invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of the present invention.

Examples of bacterial constructs include the pET series of E. coli expression vectors [Studier et al. (1990) Methods in Enzymol. 185:60-89).

In yeast, a number of vectors containing constitutive or inducible promoters can be used, as disclosed in U.S. Pat. Application No: 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art and are further described hereinbelow can also be used by the present invention.

Constructs encoding the polypeptides of the present invention are transformed into an appropriate host cell. Transformed cells are cultured under conditions, which allow for the expression of high amounts of recombinant polypeptide. Such conditions include, but are not limited to, media, bioreactor, temperature, pH and oxygen conditions that permit protein production. “Media” refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are well known to one of ordinary skill in the art.

Recovery of the recombinant polypeptide is effected following an appropriate time in culture. The phrase “recovering the recombinant polypeptide” refers to collecting the whole fermentation medium containing the polypeptide and need not imply additional steps of separation or purification. Not withstanding the above, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

As mentioned hereinabove, the biomolecular sequences of the present invention can be used to treat subjects with the above-described diseases.

The subject according to the present invention is a mammal, preferably a human which is diagnosed with one of the diseases described hereinabove, or alternatively is predisposed to having one of the diseases described hereinabove.

As used herein the term “treating” refers to preventing, curing, reversing, attenuating, alleviating, minimizing, suppressing or halting the deleterious effects of the above-described diseases.

Treating, according to the present invention, can be effected by specifically upregulating the expression of at least one of the polypeptides of the present invention in the subject.

Upregulating expression of the polypeptides of the present invention in a subject may be effected via the administration of at least one of the exogenous polynucleotide sequences of the present invention (e.g., SEQ ID NOs: 3, 7, 11, 15, 19, 23, 27, 31, 35, 39 or 43) ligated into a nucleic acid expression construct designed for expression of coding sequences in eukaryotic cells (e.g., mammalian cells). Accordingly, the exogenous polynucleotide sequence may be a DNA or RNA sequence encoding the variants of the present invention or active portions thereof.

It will be appreciated that the nucleic acid construct can be administered to the individual employing any suitable mode of administration, described hereinbelow (i.e., in-vivo gene therapy). Alternatively, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

Preferably, the promoter utilized by the nucleic acid construct of the present invention is active in the specific cell population transformed. Examples of cell type-specific and/or tissue-specific promoters include promoters, such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the trasgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

It will be appreciated that the present methodology may also be effected by specifically upregulating the expression of the splice variants of the present invention endogenously in the subject. Agents for upregulating endogenous expression of specific splice variants of a given gene include antisense oligonucleotides, which are directed at splice sites of interest, thereby altering the splicing pattern of the gene. This approach has been successfully used for shifting the balance of expression of the two isoforms of Bcl-x [Taylor (1999) Nat. Biotechnol. 17:1097-1100; and Mercatante (2001) J. Biol. Chem. 276:16411-16417]; IL-5R [Karras (2000) Mol. Pharmacol. 58:380-387]; and c-myc [Giles (1999) Antisense Acid Drug Dev. 9:213-220].

For example, interleukin 5 and its receptor play a critical role as regulators of hematopoiesis and as mediators in some inflammatory diseases such as allergy and asthma. Two alternatively spliced isoforms are generated from the IL-5R gene, which include (i.e., long form) or exclude (i.e., short form) exon 9. The long form encodes for the intact membrane-bound receptor, while the shorter form encodes for a secreted soluble non-functional receptor. Using 2′-O-MOE-oligonucleotides specific to regions of exon 9, Karras and co-workers (supra) were able to significantly decrease the expression of the wild type receptor and increase the expression of the shorter isoforms. Design and synthesis of oligonucleotides which can be used according to the present invention are described hereinbelow and by Sazani and Kole (2003) Progress in Moleclular and Subcellular Biology 31:217-239.

Alternatively or additionally, upregulation may be effected by administering to the subject at least one of the polypeptides of the present invention (e.g., recombinant or synthetic) or an active portion thereof, as described hereinabove. However, since the bioavailability of large polypeptides is relatively small due to high degradation rate and low penetration rate, administration of polypeptides is preferably confined to small peptide fragments (e.g., about 100 amino acids).

The agents of the present invention (e.g., polynucleotides, polypeptides, oligonucleotides) can be provided to the subject per se, or as part of a pharmaceutical composition where they are mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect (e.g., polynucleotides, polypeptides, oligonucleotides of the present invention).

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections. Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in a conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

It will be appreciated that treatment of the above-described diseases according to the present invention may be combined with other treatment methods known in the art (i.e., combination therapy). Thus, treatment of malignancies using the agents of the present invention may be combined with, for example, radiation therapy, antibody therapy and/or chemotherapy.

It will be appreciated that since abnormal expression (i.e., upregulation or downregulation as compared to normal state) of these biomolecular sequences may contribute to disease onset or progression or be present during the course of the disease, such biomolecular sequences can also be used as valuable diagnostic markers.

Thus, the present invention also envisages determining a level of a biomolecular sequence of the present invention (i.e., a polynucleotide or a polypeptide) in a biological sample obtained from the subject. Wherein the level determined can be correlated with predisposition to, or presence or absence of the above-described corresponding disease, thereby diagnosing predisposition to, or presence of such disease in the subject.

As used herein the term “diagnosing” refers to classifying a disease or a symptom, determining a severity of the disease, monitoring disease progression and therapeutic treatment, forecasting an outcome of a disease and/or prospects of recovery.

As used herein, the term “level” refers to expression levels of RNA and/or protein or to DNA copy number of the variants of the present invention.

A level correlatable with predisposition to, or presence or absence of a disease can be a level of a variant of the present invention in a pathological sample which is different (i.e., increased or descreased) from the level of the same variant in a normal healthy sample obtained from a similar tissue or cellular origin.

As used herein “a biological sample” refers to a sample of tissue or fluid isolated from the subject, including but not limited to, for example, plasma, serum, spinal fluid, lymph fluid, the external sections of the skin, respiratory, intestinal, and genitourinary tracts, tears, saliva, milk, blood cells, tumors, organs, and also samples of in vivo cell culture constituents.

Numerous well known tissue or fluid collection methods can be utilized to collect the biological sample from the subject in order to determine the level of DNA, RNA and/or polypeptide of the variants of the present invention in the subject.

Examples include, but are not limited to, fine needle biopsy, needle biopsy, core needle biopsy and surgical biopsy.

Regardless of the procedure employed, once a biopsy is obtained the level of the variants of the present invention can be determined and a diagnosis can thus be made.

Determining a level of the variants of the present invention can be effected using various biochemical and molecular approaches used in the art for determining gene amplification, and/or level of gene expression.

Determining the level of the variants of the present invention in normal tissues of the same origin is preferably effected along side to detect an elevated expression and/or amplification. Additionally or alternatively, determining the level of wild-type gene product (e.g., c-Met) is preferably effected along side.

Typically, detection of a nucleic acid of interest in a biological sample is effected by hybridization-based assays using an oligonucleotide probe.

The term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions. For example, an oligonucleotide probe which can be utilized by the present invention is a single stranded polynucleotide which includes a sequence complementary to a unique sequence region of a variant of the present invention (e.g., SEQ ID NO: 4).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The oligonucleotide of the present invention is of at least 10, at least 11, at least 12, at least 13, at least 14, at least 15, at least 16, at least 17, at least 18, at least 19, at least 20, at least 22, at least 25, at least 30 or at least 40, bases specifically hybridizable with the variants of the present invention.

The oligonucleotides of the present invention may comprise heterocylic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder. These can be efficiently used for in-vivo diagnosis procedures.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623, 070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi YS et al. (1993) Antisense Research and Applications, CRC Press, Boca Raton 276-278] and are presently preferred base substitutions, even more particularly when combined with 2′-O-methoxyethyl sugar modifications.

It will be appreciated that oligonucleotides of the present invention may include further modifications which increase bioavailability, therapeutic efficacy and reduce cytotoxicity. Such modifications are described in Younes (2002) Current Pharmaceutical Design 8:1451-1466.

Hybridization based assays which allow the detection of the variants of the present invention (i.e., DNA or RNA) in a biological sample rely on the use of oligonucleotide which can be 10, 15, 20, or 30 to 100 nucleotides long preferably from 10 to 50, more preferably from 40 to 50 nucleotides. It will be appreciated, though, that the length of the oligonucleotide of the present invention will greatly depend on the length and composition of the unique nucleic acid sequence region of the variant.

Hybridization of short nucleic acids (below 200 bp in length, e.g. 17-40 bp in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample (target).

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others [e.g., Kricka et al. (1992), Academic Press San Diego, Calif] can be attached to the oligonucleotides.

Traditional hybridization assays include PCR, RT-PCR, real-time PCR, RNase protection, in-situ hybridization, primer extension, Southern blot, Northern Blot and dot blot analysis.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

It will be appreciated that antisense oligonucleotides may be employed to quantify expression of a splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity [see Sazani and Kole (2003), supra].

Polymerase chain reaction (PCR)-based methods may be used to identify the presence of mRNAs of the variants of the present invention. For PCR-based methods a pair of oligonucleotides is used, which is specifically hybridizable with the polynucleotide sequences described hereinabove in an opposite orientation so as to direct exponential amplification of a portion thereof (including the hereinabove described sequence alteration) in a nucleic acid amplification reaction.

The polymerase chain reaction and other nucleic acid amplification reactions are well known in the art and require no further description herein. The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Hybridization to oligonucleotide arrays may be also used to determine expression of the variants of the present invention. Such screening has been undertaken in the BRCA1 gene and in the protease gene of HIV-1 virus [see Hacia et al., (1996) Nat Genet 1996;14(4):441-447; Shoemaker et al., (1996) Nat Genet 1996;14(4):450-456; Kozal et al., (1996) Nat Med 1996;2(7):753-759].

The nucleic acid sample, which includes the candidate region to be analyzed is isolated, amplified and labeled with a reporter group. This reporter group can be a fluorescent group such as phycoerythrin. The labeled nucleic acid is then incubated with the probes immobilized on the chip using a fluidics station. For example, Manz et al. (1993) Adv in Chromatogr 1993; 33:1-66 describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for the above-mentioned diseases both rapidly and easily.

The presence of the variants of the present invention may also be detected at the protein level. Numerous protein detection assays are known in the art, examples include, but are not limited to, chromatography, electrophoresis, immunodetection assays such as ELISA and western blot analysis, immunohistochemistry and the like, which may be effected using antibodies specific to the variants of the present invention.

Preferably used are antibodies which specifically interact with the polypeptides of the present invention and not with the protein or other isoforms thereof, for example. Such antibodies are directed, for example, to the unique sequence portions of the polypeptide variants of the present invention (e.g., SEQ ID NOs: 2, 6, 10, 14, 18, 22, 26, 30, 34, 38, 42) or to unique sequences, which bridge the common portion, which is shared with the wild-type sequence, and the unique sequence regions.

Preferably, the antibody of this aspect of the present invention specifically binds at least one epitope of the polypeptide variants of the present invention. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)₂, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)₂ is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference). It will be appreciated that the polypeptides of the present invention may be coupled with a carrier protein with high antigenicity, or the polypeptides are administrated together with a suitable adjuvant. Examples of carrier proteins include hemocyanin of Fissurellidae, keyhole limpet hemocyanin, bovine serum albumin, bovine thyroalbumin, or the like. Examples of adjuvants include Complete Freund's Adjuvant, hydroxylated aluminum gel, pertussis vaccine, or the like.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boemer et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

The diagnostic reagents described hereinabove can also be included in kits. For example a kit for diagnosing predisposition to, or presence of the above-described diseases in a subject can include an antibody directed at the unique amino acid sequence of the Met variant (SEQ ID NO: 2) in a one container and a solid phase for attaching multiple biological samples packaged in a second container with appropriate buffers and preservatives and used for diagnosis. It will be appreciated that such a kit can also include reagents which detect the level of the wild-type gene (e.g., c-Met).

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Description of the Methodology Undertaken to Uncover the Biomolecular Sequences of the Present Invention and Uses Therefor

Human ESTs and cDNAs were obtained from NCBI GenBank version 126 (Oct. 15, 2001, www.ncbi.nlm.nih.gov/dbEST) and aligned to the human genome (NCBI assembled genomic sequence from October 2001) using the LEADS clustering and assembly system as described in U.S. Pat. No. 6,625,545 and U.S. patent application Ser. No. 10/426,002.

Briefly, the software cleans the expressed sequences from repeats, vectors and immunoglobulins. It then aligns the expressed sequences to the genome taking alternatively splicing into account and clusters overlapping expressed sequences into “clusters” that represent genes or partial genes. These were annotated using the GeneCarta (Compugen, Tel-Aviv, Israel) platform. The GeneCarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

Brief description of the methodology used to obtain annotative sequence information is summarized infra (for detailed description see U.S. patent application Ser. No. 10/426,002).

The ontological annotation approach—An ontology refers to the body of knowledge in a specific knowledge domain or discipline such as molecular biology, microbiology, immunology, virology, plant sciences, pharmaceutical chemistry, medicine, neurology, endocrinology, genetics, ecology, genomics, proteomics, cheminformatics, pharmacogenomics, bioinformatics, computer sciences, statistics, mathematics, chemistry, physics and artificial intelligence.

An ontology includes domain-specific concepts—referred to, herein, as sub-ontologies. A sub-ontology may be classified into smaller and narrower categories. The ontological annotation approach is effected as follows.

First, biomolecular (i.e., polynucleotide or polypeptide) sequences are computationally clustered according to a progressive homology range, thereby generating a plurality of clusters each being of a predetermined homology of the homology range.

Progressive homology is used to identify meaningful homologies among biomolecular sequences and to thereby assign new ontological annotations to sequences, which share requisite levels of homologies. Essentially, a biomolecular sequence is assigned to a specific cluster if displays a predetermined homology to at least one member of the cluster (i.e., single linkage). A “progressive homology range” refers to a range of homology thresholds, which progress via predetermined increments from a low homology level (e.g. 35%) to a high homology level (e.g. 99%).

Following generation of clusters, one or more ontologies are assigned to each cluster. Ontologies are derived from an annotation preassociated with at least one biomolecular sequence of each cluster; and/or generated by analyzing (e.g., text-mining) at least one biomolecular sequence of each cluster thereby annotating biomolecular sequences.

The hierarchical annotation approach—“Hierarchical annotation” refers to any ontology and subontology, which can be hierarchically ordered, such as, a tissue expression hierarchy, a developmental expression hierarchy, a pathological expression hierarchy, a cellular expression hierarchy, an intracellular expression hierarchy, a taxonomical hierarchy, a functional hierarchy and so forth.

The hierarchical annotation approach is effected as follows.

First, a dendrogram representing the hierarchy of interest is computationally constructed. A “dendrogram” refers to a branching diagram containing multiple nodes and representing a hierarchy of categories based on degree of similarity or number of shared characteristics.

Each of the multiple nodes of the dendrogram is annotated by at least one keyword describing the node, and enabling literature and database text mining, such as by using publicly available text mining software. A list of keywords can be obtained from the GO Consortium (www.geneontlogy.org). However, measures are taken to include as many keywords, and to include keywords which might be out of date. For example, for tissue annotation, a hierarchy is built using all available tissue/libraries sources available in the GenBank, while considering the following parameters: ignoring GenBank synonyms, building anatomical hierarchies, enabling flexible distinction between tissue types (normal versus pathology) and tissue classification levels (organs, systems, cell types, etc.).

In a second step, each of the biomolecular sequences is assigned to at least one specific node of the dendrogram.

The biomolecular sequences can be annotated biomolecular sequences, unannotated biomolecular sequences or partially annotated biomolecular sequences.

Annotated biomolecular sequences can be retrieved from pre-existing annotated databases as described hereinabove.

For example, in GenBank, relevant annotational information is provided in the definition and keyword fields. In this case, classification of the annotated biomolecular sequences to the dendrogram nodes is directly effected. A search for suitable annotated biomolecular sequences is performed using a set of keywords which are designed to classify the biomolecular sequences to the hierarchy (i.e., same keywords that populate the dendrogram) In cases where the biomolecular sequences are unannotated or partially annotated, extraction of additional annotational information is effected prior to classification to dendrogram nodes. This can be effected by sequence alignment, as described hereinabove. Alternatively, annotational information can be predicted from structural studies. Where needed, nucleic acid sequences can be transformed to amino acid sequences to thereby enable more accurate annotational prediction.

Finally, each of the assigned biomolecular sequences is recursively classified to nodes hierarchically higher than the specific nodes, such that the root node of the dendrogram encompasses the full biomolecular sequence set, which can be classified according to a certain hierarchy, while the offspring of any node represent a partitioning of the parent set.

For example, a biomolecular sequence found to be specifically expressed in “rhabdomyosarcoma”, will be classified also to a higher hierarchy level, which is “sarcoma”, and then to “Mesenchimal cell tumors” and finally to a highest hierarchy level “Tumor”. In another example, a sequence found to be differentially expressed in endometrium cells, will be classified also to a higher hierarchy level, which is “uterus”, and then to “women genital system” and to “genital system” and finally to a highest hierarchy level “genitourinary system”. The retrieval can be performed according to each one of the requested levels.

Annotating gene expression according to relative abundance—Spatial and temporal gene annotations are also assigned by comparing relative abundance in libraries of different origins. This approach can be used to find genes, which are differentially expressed in tissues, pathologies and different developmental stages. In principal, the presentation of a contigue in at least two tissues of interest is determined and significant over or under representation of the contigue in one of the at least two tissues is assessed to identify differential expression. Significant over or under representation is analyzed by statistical pairing.

Annotating spatial and temporal expression can also be effected on splice variants. This is effected as follows. First, a contigue which includes exonal sequence presentation of the at least two splice variants of the gene of interest is obtained. This contigue is assembled from a plurality of expressed sequences;

Then, at least one contigue sequence region, unique to a portion (i.e., at least one and not all) of the at least two splice variants of the gene of interest, is identified Identification of such unique sequence region is effected using computer alignment software.

Finally, the number of the plurality of expressed sequences in the tissue having the at least one contigue sequence region is compared with the number of the plurality of expressed sequences not-having the at least one contigue sequence region, to thereby compare the expression level of the at least two splice variants of the gene of interest in the tissue.

Data concerining therapies, indications and possible pharmacological activities of the polypeptides of the present invention was obtained from PharmaProject (PJB Publications Ltd 2003 http://www.pjbpubs.com/cms.asp?pageid=340) and public databases, including LocusLink (http://www.genelynx.org/cgi-bin/resource?res=locuslink) and Swissprot (http://www.ebi.ac.uk/swissprot/index.html). Functional structural analysis of the polypeptides of the present invention was effected using Interpro domain analysis software (Interpro default parameters, the analyses that were run are HMMPfam, HMMSmart, ProfileScan, FprintScan, and BlastProdom). Subecllular localization was analysed using ProLoc software (Einat Hazkani-Covo, Erez Y. Levanon, Galit Rotman, Dan Graur, Amit Novik. Evolution of multicellularity in metazoa: comparative analysis of the subcellular localization of proteins in Saccharomyces, Drosophila and Caenorhabditis. Cell Biology International (in press).

### Example 2

### Met- Hepatocyte Growth Factor Receptor

The protein product of c-met oncogene (Swissprot Locus No. MET_HUMAN), is a tyrosine kinase receptor for hepatocyte growth factor (HGF, Swissprot Locus No. HGF_HUMAN), also known as scatter factor (SF). HGF/SF was identified independently as both a growth factor for hepatocytes and as a fibroblast-derived cell motility factor, or scatter factor. The mature HGF is a disulfide-linked heterodimer composed of an α-chain and a β-chain deriving from a pre-pro HGF polypeptide that is proteolytically cleaved to yield the mature active HGF. HGF is produced predominantly by mesenchymal cells and acts primarily on Met-expressing ephithelial cells in an endocrine and/or paracrine fashion. The HGF/SF-Met complex induces a wide range of biological events, including proliferation, scattering, invasion, branching morphogenesis, transformation and angiogenesis. This wide array of biological processes is mediated by the HGF-Met signaling involving interactions between mesenchymal and epithelial cells. Such paracrine signaling is vital to embryogenesis and contributes to kidney and mammary gland formation, migration and development of muscle and neuronal precursors, and liver and placenta development.

Binding of HGF to the extracellular domain of Met triggers autophosphorylation of specific tyrosine residues in the intracellular region of Met.

Specifically, phosphorylation of two tyrosine residues (Tyr1234 and Tyr1235) located within the activation loop of the tyrosine kinase domain activates the intrinsic kinase activity of the receptor, while phosphorylation of two tyrosine residues (Tyr1349 and Tyr1356) located in the C-terminus of Met activates a multisubstrate docking site, recruiting downstream signaling molecules and adaptor proteins thereby amplifying the cellular responses from HGF to multiple distinctive pathways.

While HGF/SF-Met signaling plays a key role during normal development (embryonic development, wound healing and tissue regeneration) this signaling pathway has been implicated in tumor development and progression. In particular, abnormal HGF-Met signaling was shown to play a significant role in promoting tumor cell invasion and metastasis. c-Met mutations have been described in hereditary and sporadic human papillary renal carcinomas and have been reported in ovarian cancer, childhood hepatocellular carcinoma, metastatic head and neck squamous cell carcinomas, and gastric cancer. c-Met is also over-expressed in both small cell lung cancer and non-small cell lung cancer. In view of its critical role in oncogenesis, various inhibition strategies have been employed to therapeutically target Met receptor tyrosine kinase.

Chromosomal Location and Structural Information

The human Met gene, which includes 21 exons, is located on chromosome 7 band 7q21-q31 and spans more than 120 kb in length.

The primary Met transcript produces a 150 kDa polypeptide (1390 amino acids) that is partially glycosylated to produce a 170 kDa precursor protein. This 170 kDa precursor is further glycosylated and then cleaved into a 50 kDa α-chain and a 140 kDa β-chain which are disulfide-linked. The α-subunit of the mature Met heterodimer is highly glycosylated and is entirely extracellular, while the β-subunit contains a large extracellular region, a membrane spanning segment, and an intracellular tyrosine kinase domain.

Met is the prototypic member of a subfamily of heterodimeric receptor tyrosine kinases which include Met, Ron, and Sea. Members of the Met receptor subfamily have been shown to share homology with semaphorins and semaphorin receptors (plexin), which play a role in cell scattering. All semaphorins contain a conserved 500 amino acid extracellular domain (Sema domain) in which resides the cysteine-rich Met related sequence (MRS) which minimal consensus is C—X(5-6)—C—X(2)—C—X(6-8)—C—X(2)—C—X(3-5)-C. The extracellular portions of Met, Ron, and Sea contain a region of homology to semaphorins including the N-terminal Sema domain and the MRS. Other domains identified in the extracellular portion of Met are the PSI domain and the IPT/TIG repeat domain. The PSI domain is found in plexins, semaphorins and integrins while the IPT repeats (also known as TIG domains) are found within immunoglobulin, plexins and transcription factors. The C-terminus intracellular tyrosine kinase domain shares homology with Ron and Sea.

Biological Function

1. Proliferation—Aside from hepatocytes, HGF is also an important growth factor for cells of various origins, including epithelial cells, neurons and placental cytotromphoblasts. The mitogenic effect of HGF is also crucial in regenerative processes following tissue injury.

2. Scattering—The process of cell scattering can be divided into three phases, essentially, cell spreading, cell-cell dissociation, and cell migration. As mentioned above, HGF was discovered as a secretory product of fibroblasts and smooth muscle cells that induces dissociation and motility of epithelial cells. HGF/SF is able to induce cell dissociation and mutual repulsion in a similar manner to semaphorins.

3. Angiogenesis—HGF acts as a potent angiogenic molecule by directly acting on vascular endothelial cells. HGF stimulation of vascular endothelial cells promotes migration, proliferation, protease production, invasion, and organization into capillary-like tubes. HGF can also promote the expression of angiogenic factors by tumor cells. HGF induces a dose-dependent increase in the angiogenic cytokines IL-8 and vascular endothelial growth factor (VEGF) produced by head and neck squamous cell carcinoma cell lines.

4. Cell motility—The key events regulating cell motility are polymerization of actin, formation of actin stress fibers, and focal adhesion formation. Many studies have shown that HGF-Met signaling increases the motility of epithelial cells.

5. Morphogenesis—HGF has been shown to induce branching morphogenesis of kidney, mammary and bile ductular cells. In response to HGF, Met expressing cells form branches in three-dimensional matrigel or tubule-like structures in collagen gels. This process is mediated through changes in cell shape, asymmetric polarization of the cells in the direction of branching, branch elongation, cell-cell contact, cell-ECM communication, ECM remodeling, controlled proteolysis and cell motility.

6. Invasion and metastasis—HGF/SF-Met signaling has been strongly implicated in the promotion of the invasive/metastatic tumor phenotype. An HGF stimulated pathway involving MAPK1/2 signaling is important in the up-regulation of expression of the serine protease urokinase (uPA) and its receptor (uPAR), resulting in an increase of uPA on the cell surface. Certain components of the ECM can be directly degraded by uPA, and more importantly, uPA cleaves plasminogen into the broader-specificity protease plasmin, which is able to efficiently degrade several ECM and basement membrane (BM) components. Plasmin also activates metalloproteinases, which have potent ECM/BM degrading abilities. HGF has been reported to promote attachment of tumor cells to endothelium, an important step in the metastatic cascade. This activity may be mediated by HGF induced up-regulation of CD44 expression on endothelium cells, and an induced integrin expression on tumor cells.

Abnormal Activity and/or Expression of Met-HGF in Cancer

Aberrant c-Met signaling has been described in a variety of human cancers (solid tumors and hematologic malignancies). Mutations in c-Met, over-expression of c-Met and/or HGF, and expression of c-Met and HGF by the same cell can all contribute to tumorigenesis. Cell lines with uncontrolled c-Met activation via one of these mechanisms are both highly invasive and metastatic. Increased c-Met and/or HGF expression by human tumor cells is often associated with high tumor grade and poor prognosis.

References

Maulik et al. 2002. Cytokine and Growth Factor Reviews. 13:41-59.

Zhang et al. 2003. Journal of Cellular Biochemistry. 88:408-417.

Furge et al. 2000. Oncogene. 19:5582-5589.

To et al. 1998. Oncology Reports. 5:1013-1024.

Trusolino et al. 1998. The FASEB Journal. 12:1267-1280.

Comoglio et al. 2002. The Journal of Clinical Investigation. Vol. 109, No. 7, Pp. 857-862.

Danilkovitch-Miagkova et al. 2002. The Journal of Clinical Investigation. Vol. 109, No. 7, Pp. 863-867.

Wajih et al. 2002. Circulation Research. 90:46-52.

Mark et al. 1992. The Journal of Biological Chemistry. Vol 267, No. 36, Pp. 26166-26171.

Met Splice Variant-Structure

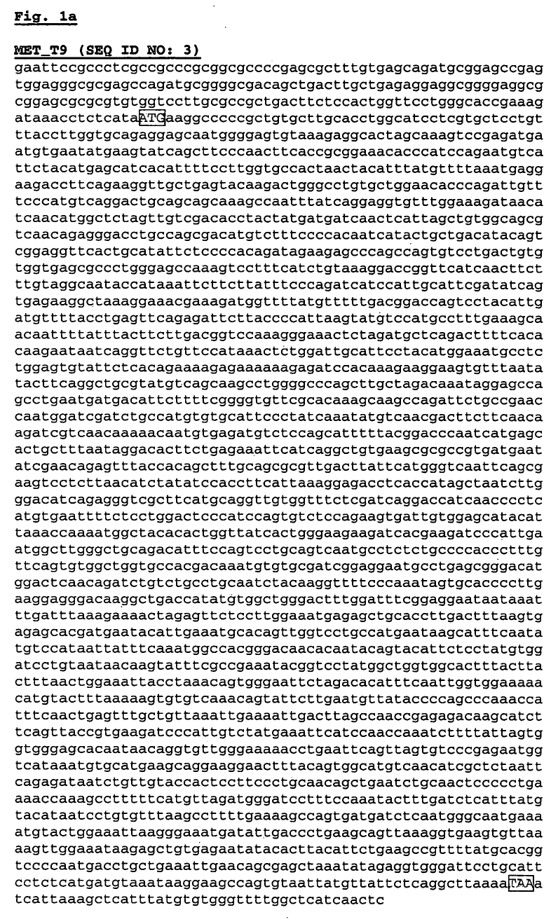

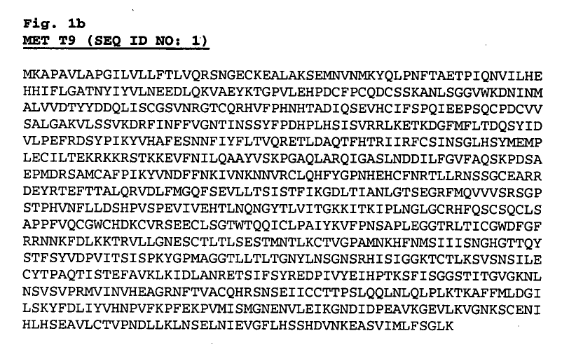

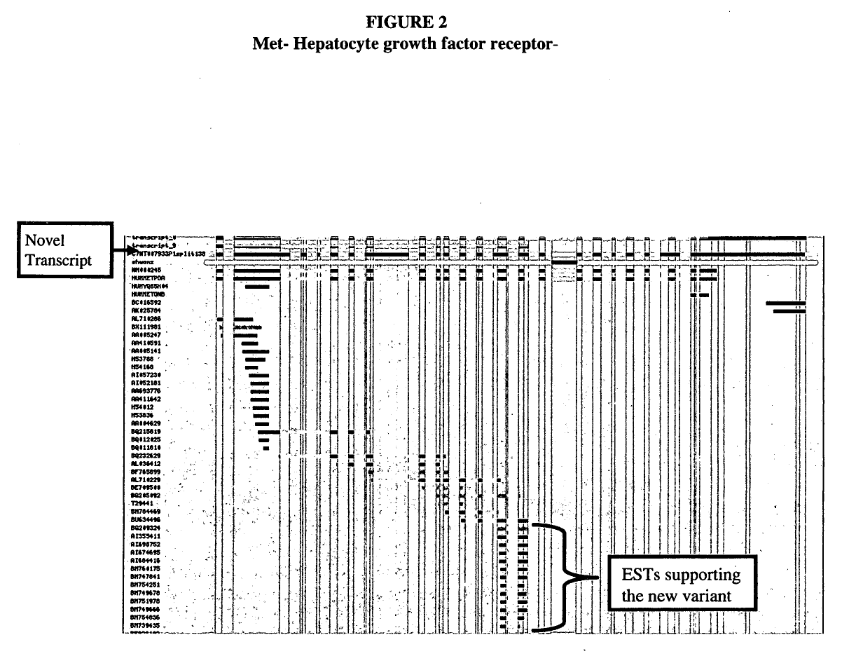

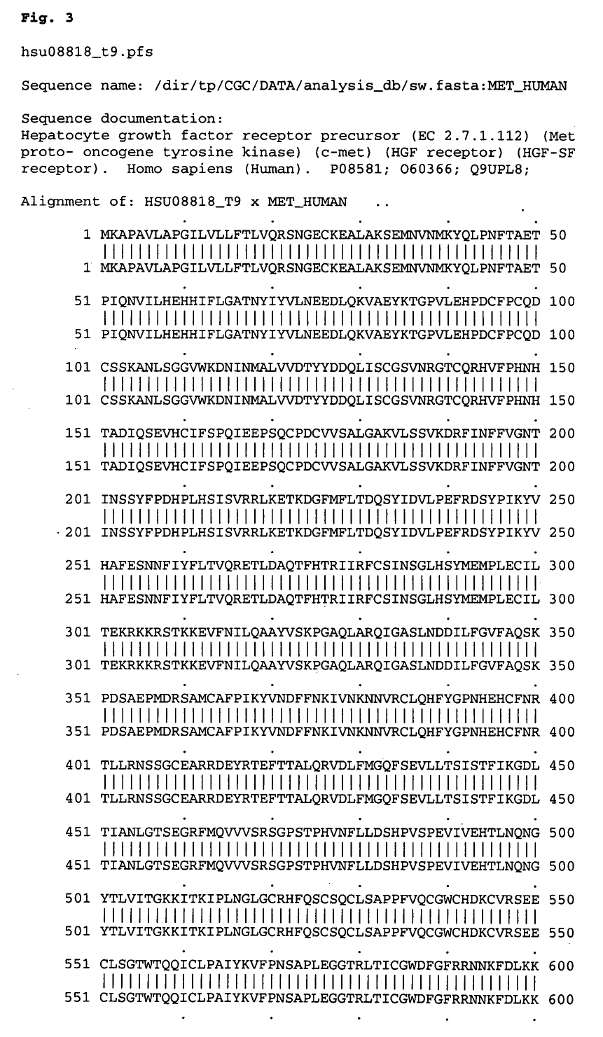

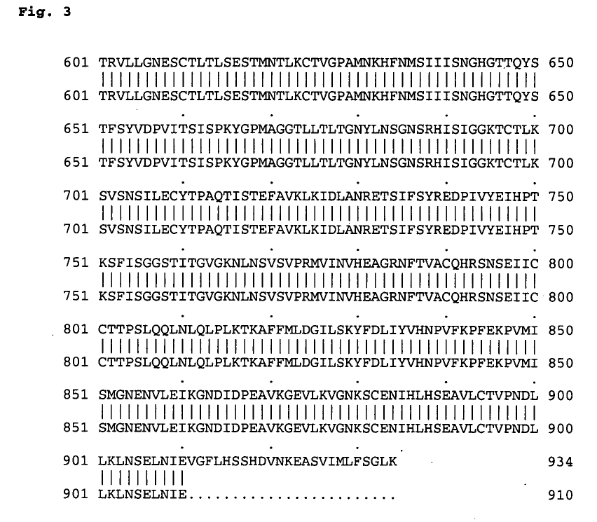

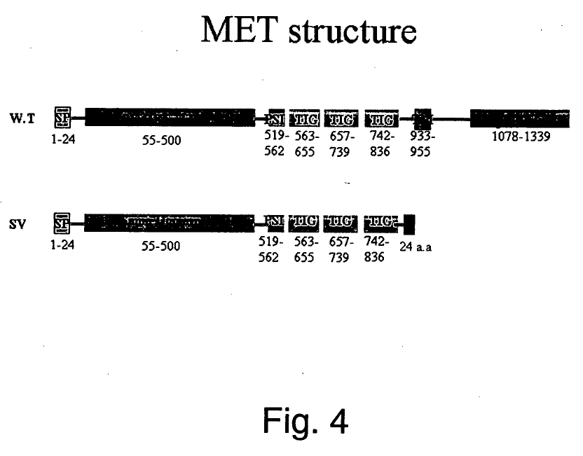

The present inventors have uncovered a novel splice variant of Met (SEQ ID NOs: 1, 3, FIGS. 1a,b-4) by LEADS clustering and assembly algorithm and the annotation process, as described above (Example 1). This splice variant results from alternative splicing of the c-Met gene, thereby causing an extension of exon 12 (the last exon before the transmembrane region encoding exon) leading to an insertion of a stop codon and the generation of a truncated Met protein which terminates just before the transmembrane domain. Met splice variant has an open reading frame (ORF) of 934 amino acids: 910 amino acids of the wild-type (w.t.) Met protein and a unique sequence of 24 amino acids at the C-terminus of the protein. It contains nearly the complete extracellular portion of Met (910 amino acids of 933 of the w.t protein) and therefore comprises all its structural domains (the Sema, PSI and TIG domains). It is predicated to be a secreted protein since it retains the original N-terminal signal peptide (amino acids 1-24) and lacks the transmembrane domain (amino acids 933-955 of the w.t.).

Therapeutic and Diagnostic Applications for the Met Splice Variant of the Present Invention

Met splice variant of the present invention can serve as a antagonist (i.e., inhibitor) of Met-HGF interaction. It contains the extracellular region of Met, the HGF binding site, and therefore it is likely to bind HGF. Met extracellular region has been shown previously to bind HGF with a high affinity (comparable to the membrane bound receptor). Met splice variant can inhibit Met-HGF signaling by competing with the membrane-bound receptor for the ligand-HGF, thus preventing HGF binding to the cell surface receptor and as a consequence blocking Met activation and its signaling pathway.

Because of the overwhelming evidence favoring the role of aberrant HGF-Met signaling in the pathogenesis of various human cancers, endogenous and exogenous inhibitors of this signaling pathway such as Met splice variant may be used as valuable therapeutic tools in the treatment of cancers such as, hereditary and sporadic papillary renal carcinoma, breast cancer, ovarian cancer, childhood hepatocellular carcinoma, metastatic head and neck squamous cell carcinomas, lung cancer (e.g., non-small cell lung cancer, small cell lung cancer), prostate cancer, pancreatic cancer, gastric cancer and other diseases such as diabetic retinopathy.

In addition to its therapeutic potential, Met splice variant may be used for diagnosing cancer. Aberrant c-Met signaling, which results from mutations in the Met sequence or over-expression of c-Met, has been described in a variety of human cancers. Furthermore, increased c-Met expression by human tumor cells is often associated with high tumor grade and poor prognosis.

### Example 3

### Interleukin-6 (IL-6)

Background

IL-6 (Swissprot Locus No. IL6_HUMAN) was originally identified as an antigen-nonspecific B-cell differentiation factor in culture media of mitogen- or antigen-stimulated peripheral blood mononuclear cells that induced B cells to produce immunoglobulins and was named B-cell stimulatory factor 2 (BSF-2).

IL-6 is a pleiotropic cytokine with a wide range of biological activities in immune regulation, hematopoiesis, inflammation, and oncogenesis. The cytokine is produced by various types of lymphoid and nonlymphoid cells, such as T cells, B cells, monocytes, fibroblasts, keratinocytes, endothelial cells, mesangial cells, and several tumor cells. It induces growth of T cells and differentiation of cytotoxic T cells by augmenting the expression of IL-2 receptor and IL-2. IL-6 acts synergistically with IL-3 to support the formation of multilineage blast cell colonies in hematopoiesis. It also induces differentiation of macrophages, megakaryocytes and osteoclasts. In the acute-phase reaction, this cytokine stimulates hepatocytes to produce acute-phase proteins such as C-reactive protein (CRP), fibrinogen, α₁-antitrypsin and serum amyloid A and it simultaneously suppresses albumin production. It causes leukocytosis and fever when administered in vivo and also acts as a growth factor for renal mesangial cells, epidermal keratinocytes and various types of tumor cells, for example, in plasmacytoma, multiple myeloma and renal cell carcinoma.

Although IL-6 has pleiotropic effects on various target cells, some of these biological activities are also mediated by other cytokines, such as leukemia inhibitory factor (LIF) and oncostatin M (OSM). The pleiotropy and redundancy of IL-6 functions can be explained by the unique composition of its receptor signaling system. Two components of IL-6 receptor (IL-6R) were identified, an 80-kDa IL-6-binding protein (\tilde{α} chain) and a common 130-kDa signal transducer known as gp130 (β chain). The gp130 subunit is shared by receptors for other cytokines of the IL-6 superfamily, such as ciliary neurotrophic factor (CNTF), LIF, OSM, IL-11, and cardiotrophin-1 receptors. Although IL-6 cannot directly bind to gp130, it can bind to IL-6R to thereby generate the high-affinity complex of IL-6/IL-6R/gp130. Mutagenesis studies have identified three distinct regions on the surface of IL-6 which specifically interact with the respective receptors: site I interacts with IL-6Rα; site II, which is common to all IL-6-type cytokines, interacts with the cytokine-binding module of gp130 and site III interacts with the second signaling receptor (another gp130, in the case of IL-6). The signal transduction through gp130 is mediated by two pathways: the JAK-STAT (Janus family tyrosine kinase-signal transducer and activator of transcription) pathway and the Ras mitogen-activated protein kinase (MAPK) pathway.

Clinical Application

IL-6 have been shown to play key roles in various disease conditions, such as inflammatory, autoimmune, and malignant diseases. Uncontrolled IL-6 overproduction appears to be responsible for the clinical symptoms and abnormal laboratory findings in Rheumatoid arthritis (RA). The ability of IL-6 to induce differentiation of B-cells suggests that overproduction of IL-6 is responsible for the increase in serum γ-globulin and the emergence of rheumatoid factors in RA. As a hepatocyte-stimulating factor, IL-6 causes an increase in CRP, serum amyloid A, and erythrocyte sedimentation rate and a decrease in serum albumin. On the other hand, IL-6 as a megakaryocyte differentiation factor causes thrombocytosis. The ability of IL-6 in the presence of soluble IL-6R to induce bone absorption, suggests that IL-6 may be involved in the osteoporosis and destruction of bone and cartilage associated with RA. In fact, a large amount of IL-6 has been observed in both sera and synovial fluids from the affected joints of patients with RA. Blockade of the IL-6 signaling may thus constitute a new therapeutic strategy for RA. In addition to RA, IL-6 was found to be involved in various diseases such as Castleman's disease, Crohn's disease, multiple myeloma/plasmacytoma, mesangial proliferative glomerulonephritis, psoriasis and Kaposi's sarcoma. Thus RA and these other diseases could be targets for IL-6 inhibitors.

References

Simpson et al. 1997. Protein Science. 6:929-955.

Jones et al. 2001. The FASEB Journal. 15:43-58.

Hirano T. 1998. Intern. Rev. Immunol. Vol. 16, Pp. 249-284.

Heinrich et al. 2003. Biochemical Society. 374 :1-20.

Bihl et al. 2002. American Journal Of Respiratory Cell and Molecular Biology. Vol. 27, pp. 48-56.

Naka et al. 2002. Arthritis Research. Vol 4 Suppl 3:S233-S242.

IL-6 Splice Variant-Structure

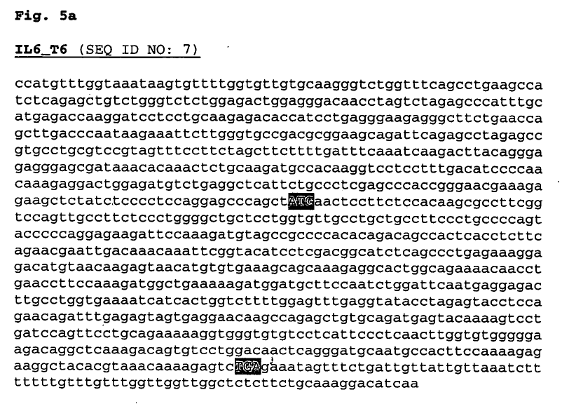

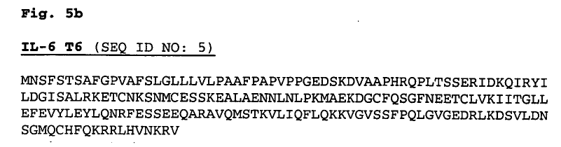

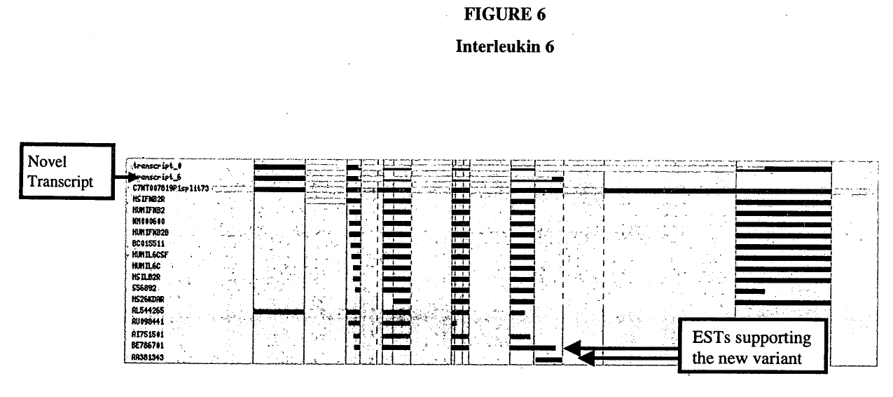

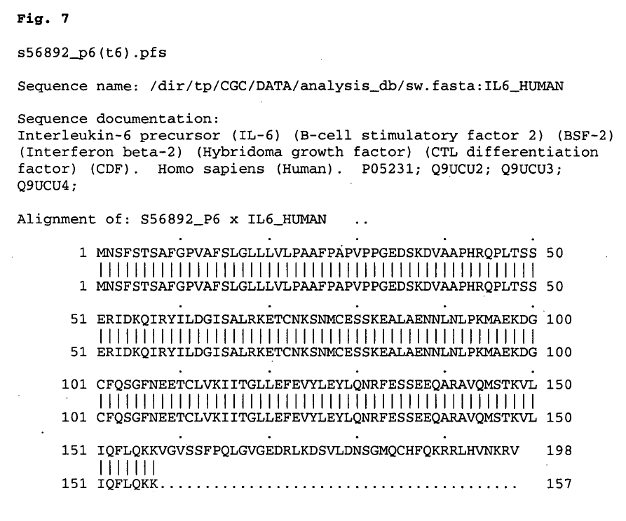

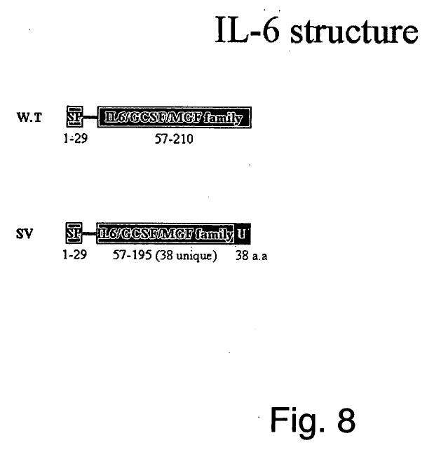

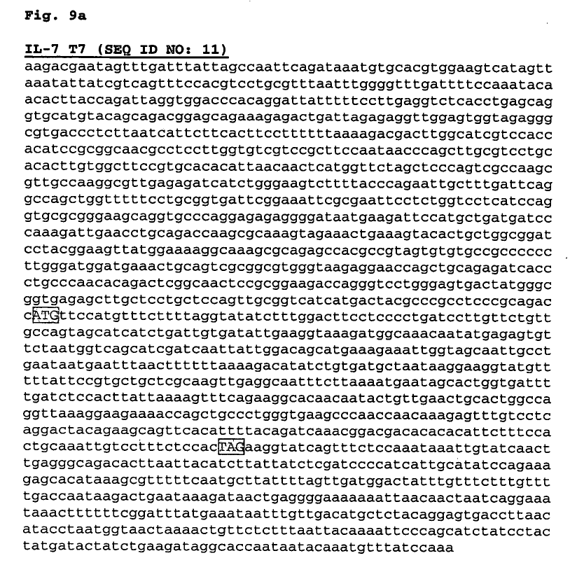

A novel splice variant of IL-6 (SEQ ID NOs: 5 and 7, FIGS. 5a-b-8) was uncovered by LEADS clustering and assembly algorithm and the annotation process, as described above (Example 1). IL-6 splice variant results from alternative splicing of the IL-6 gene, thus causing an extension of exon 4, leading to an insertion of a stop codon and the generation of a truncated protein. IL-6 splice variant encodes a 198 amino acids long protein which contains the N-terminal signal sequence (residues 1-29), most of the IL6/GCSF/MGF family domain and a unique sequence of 41 amino acids at the C-terminus of the protein.

Therapeutic Applications for the IL-6 Splice Variant of the Present Invention

The IL-6 splice variant of the present invention contains the N-terminus 157 amino acids of the wild-type while lacking the last 50 amino acids of the protein. This splice variant may serve as an antagonist of IL-6 by several mechanisms. For example, it can bind only to IL-6Rα while abolishing the binding to gp130. Since gp130 is the signaling subunit of the IL-6R complex, such variant will not be able to activate the receptor. By binding to the receptor without activating it, IL-6 splice variant might have the potential to serve as an antagonist of IL-6 signaling.

Disregulation of IL-6 production has been implicated in the pathogenesis of a variety of diseases, including plasmacytoma/myeloma and several chronic inflammatory proliferative diseases. Blocking of IL-6 signaling by inhibitors such as this IL-6 splice variant can thus have an important therapeutic potential for the treatment of Rhematoid arthritis, Castleman's disease, Crohn's disease, multiple myeloma/plasmacytoma, mesangial proliferative glomerulonephritis, psoriasis and Kaposi's sarcoma.

### Example 4

### Interleukin-7 (IL-7)

Background

The IL-7 cytokine (Swissprot Locus No. IL7_HUMAN) was originally identified as a growth factor for murine B cell progenitors and was isolated from bone marrow stromal cells. Subsequently, it was demonstrated that IL-7 plays a crucial role in normal B and T cell lymphopoiesis. It acts as a differentiation and proliferation factor of B cells and as a survival factor of activated T cells. Receptors for IL-7 have been found on cells of both the lymphoid and myeloid lineages. A heterodimeric IL-7R complex is composed of two subunits, a unique α subunit and a p64 γ subunit, which is common to the receptors for IL-2, IL-4, IL-9 and IL-15. While IL-7R expression is important for early pre-B and pro-B cell development, mature B cells lack expression of high affinity receptor and demonstrate no proliferative response to IL-7. In addition to its expression on immature B cells, IL-7R has been identified also on thymocyte and on most mature T cells, wherein the receptor is transiently down-regulated upon activation. IL-7 signaling involves a number of nonreceptor tyrosine kinase pathways that associate with the cytoplasmic tail of the receptor. These include the Janus kinase/signal transducer and activator of transcription (Jak/STAT) pathway, phosphatidylinositol 3-kinase (P13-kinase), and Src family tyrosine kinases.

Clinical Applications

Due to the numerous effects of IL-7 on mature T cells it may modulate immune responses in infectious or cancerous disease. Systemic administration of IL-7 can be used as an anti-cancer therapy by enhancing the immune responses against tumor through a variety of mechanisms. In addition, IL-7 combined with other factors, such as GM-CSF, can enhance the generation of mature monocyte-derived dendritic cells. Furthermore, IL-7, along with other cytokines, may contribute to the induction of a type 1 immune response and LAK cells. Finally, by diminishing TGF-β production, IL-7 can potentially down-regulate one mechanism through which tumors suppress local immune responses. In contrast, IL-7 stimulates the growth of pre-B and T acute lymphoblastic leukemia cells in vitro. It also induces proliferation of chronic lymphocytic leukemia cells and acute myelogenous leukemia cells, as well as cells from patients with Sezary syndrome. IL-7R is expressed on the majority of neoplastic lymphoid cells and on a subset of myeloid neoplasms. The demonstration of IL-7 secretion by neoplastic B lymphocytes from patients with Burkitt's lymphoma, Sezary leukemia cells, Hodgkin's lymphoma cells and normal keratinocytes suggests the possibility of both autocrine and paracrine growth-stimulatory mechanisms for IL-7 in neoplastic diseases. Therefore inhibiting IL-7 signaling might have a therapeutic potential in cancer therapy.

References

Cosenza et al. 2000. Protein Science. 9:916-926.

VanderSpek et al. 2002. Cytokine. Vol. 17, No. 5, Pp. 227-233.

Gorgun et al. 2002. Cytokine. Vol. 20, No. 1, Pp 17-22.

Fry et al. 2002. Blood. Vol. 99, No. 11, Pp. 3892-3904.

Splice Variant Structure

The present inventors uncovered two novel splice variants of IL-7, variant T7 (SEQ ID NOs: 9, 11) and variant T8 (SEQ ID NO: 13, 15) by applying LEADS clustering and assembly algorithm and the annotation process, as described above (Example 1).

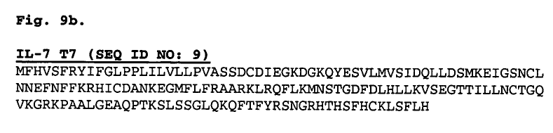

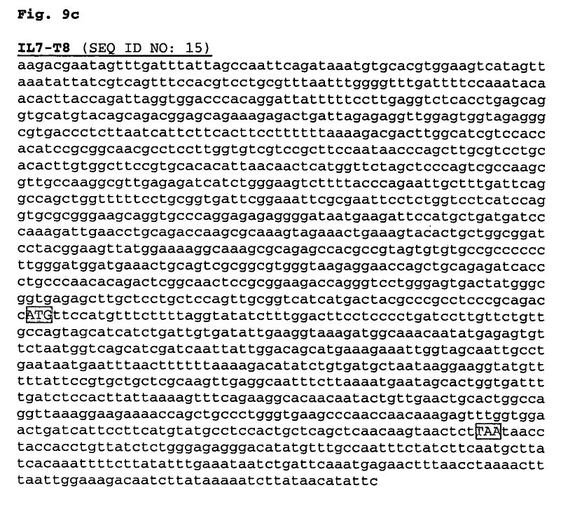

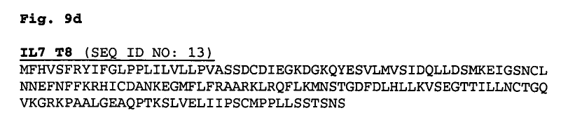

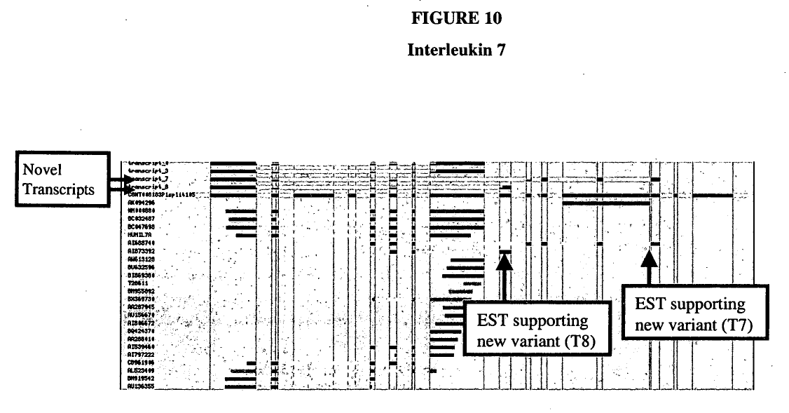

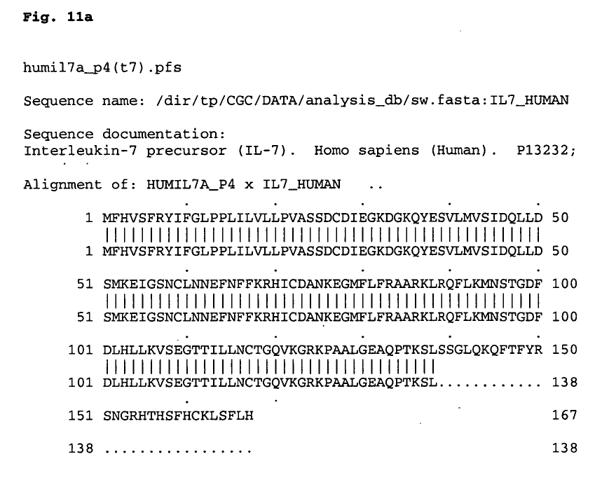

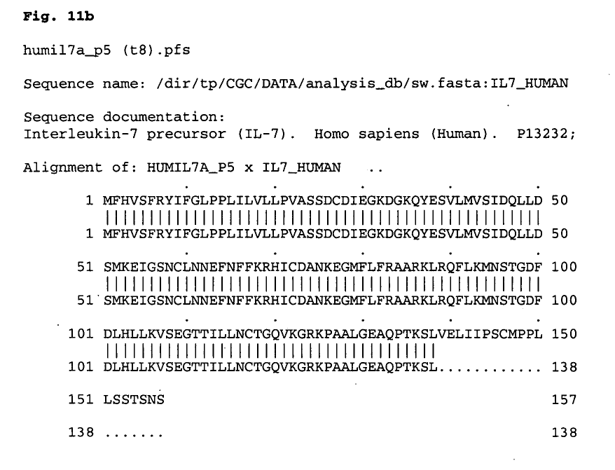

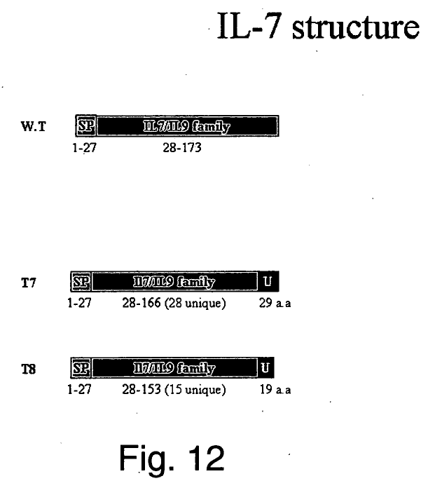

IL-7 splice variant T7 (see FIGS. 9a-b-12) results from alternative splicing of the IL-7 gene, involving the skipping of exon 6 (the last exon of wild-type IL-7) into a new last exon and 3′ UTR, and the generation of a protein with a different C-terminus. IL-7 splice variant T7 encodes a 167 amino acids long protein which contains the N-terminal signal sequence (residues 1-27), an almost the complete IL-7/IL-9 family domain and a unique sequence of 29 amino acids at the C-terminus. IL-7 splice variant T8 (see FIGS. 9c-d-12) results from alternative splicing of the IL-7 gene, involving the skipping of exon 6 (the last exon of wild-type IL-7) into a new last exon and 3′ UTR, and the generation of a protein with a different C-terminus. IL-7 splice variant T8 encodes a 157 amino acids long protein which contains the N-terminal signal sequence (residues 1-27), most of the IL-7/IL-9 family domain and a unique sequence of 19 amino acids at the C-terminus.

Therapeutic applications for the IL-7 splice variants of the present invention IL-7 splice variants contain the N-terminus 138 amino acids of the wild-type IL-7 and a unique sequences at the C terminus of the proteins. These splice variants can modulate IL-7 signaling and therefore may be used as valuable therapeutic tools in the treatment of cancers, such as acute and chronic lymphocytic leukemia, acute myelogenous leukemia, Sezary's syndome, Burkitt's lymphoma and Hodgkin's disease, and in constructing a spectrum of lymphoid cell types following radiotherapy or chemotherapy.

### Example 5

### Tumor Necrosis Factor Receptor-9/4-1BBR

Background

T cells receiving signals via the Ag-specific TCR require a second, costimulatory signal to stabilize cytokine mRNA and induce the expression of anti-apoptotic proteins. The most well-studied costimulatory pathway involves the binding of two members of the Ig supergene family B7-1 (CD80) and B7-2 (CD86), present on APCs, to T cell counter receptors CD28, and CTLA-4(CD 152). Similarly, members of the nerve growth factor/TNF superfamily can function as costimulatory molecules. These include 4-1BB receptor (CDw137), CD30, OX40, and CD40 ligand (CD154). The 4-1BB receptor (Swissprot Locus No. TNR9_HUMAN) is an inducible type I membrane protein expressed on activated cytolytic and helper T cells as well as NK cells.

The ligand for 4-1BB receptor is 4-1BB ligand (4-1BBL), which is expressed on activated APCs including activated B cells, activated macrophages and mature dendritic cells. This expression pattern suggested that pairing of 4-1BBR and 4-1BBL is important for interactions between APCs and T cells, which play a role in the process of antigen presentation. Signals delivered by the 4-1 BB receptor can induce T cells to produce IL-2, proliferate and differentiate, as well as protect T cells from activation-induced cell death. Despite the expression of 4-1BBR on both CD4+ and CD8+ T cells, 4-1BBR has been reported to predominantly affect CD8+ T cell responses. 4-1BBR was shown to regulate both clonal expansion and survival of CD8+T cells. As is the case for other TNFR superfamily members, 4-1BBR uses adaptor molecules called tumor necrosis factor receptor-associated factors (TRAFs) to transduce a downstream signal. The cytoplasmic domain of 4-1BBR is able to associate specifically with TRAF1, TRAF2, and TRAF3. Accumulating evidence indicates that there exists a signal transduction pathway via the 4-1BB ligand as well as via the 4-1BB receptor. 4-1BBR is able to replace CD28 in stimulating high-level IL-2 production by resting T cells in the absence of CD28. Since 4-1BBR must be up-regulated before providing costimulatory signals for T cells, in contrast to CD28 which is expressed constitutively, 4-1BBR may play a major role in the later stages of the immune response. Thus, it is plausible to hypothesize that CD28 and 4-1BBR play sequentially differential roles in the stages of the immune response: CD28 is more important in the induction stages of the immune response while 4-1BBR is more important in perpetuating the immune response. This task of 4-1BBR may be achieved by providing a survival signal as well as a costimulatory signal for T cells.

Clinical Applications

Administration of agonistic anti-4-1BBR antibodies has been shown to eradicate established large tumors in mice. Interestingly, anti-4-1BBR-mediated tumor elimination is a complex process which requires CD4+ T cells and NK cells as well as CD8+T cells. Thus, it seems that the immune response induced by anti-4-1BB antibodies augment tumor-specific cytotoxic activity of CD8+T cells, which is regulated by CD4+ T cells and NK cells. Similarly, the introduction of 4-1BBL into tumor cells confers full immunogenicity and thus enhances the amplification of an anti-tumor immune response. Although 4-1BB was shown to effect CD8+cells preferentially, there is accumulating evidence that 4-1BBR is implicated in immune responses mediated by CD4+ T cells, including alloimmune responses and inflammation. As with CD8+ T cells, signaling through 4-1BBR appears to promote cell proliferation and survival of CD4+ T cells. This suggested that intervention in the 4-1BBR costimulatory pathway could provide an immunotherapeutic approach to the treatment of inflammatory diseases. It has been shown that in vivo blocking of 4-1BB/4-1BBL interactions, by administration of anti 4-1BBL monoclonal antibody, significantly decrease the myocardial inflammation, induced by coxackievirus B3, and prevented the herpetic stromal keratitis (HSK) induced by HSV-1. Contrary to these observations, administration of agonistic anti-4-1BBR monoclonal antibody blocked the disease progression of spontaneous systemic lupos erythematosus (SLE). In general, it was suggested that agonistic anti 4-1 BBR monoclonal antibody may be a valid therapeutic approach to treat Th2-mediated autoimmune diseases such as SLE, RA, ulcerative colitis, whereas tools to block the 4-1BBR costimulatory pathway such as anti 4-1BBL monoclonal antibody may provide immuno-therapy to treat Th1-mediated inflammatory diseases such as MS and Crohn's disease and to prevent rejection of organ transplant. Agonistic anti 4-1BBR monoclonal antibody may also be used as an immunotherapeutic agent to eradicate tumor or viral infection.

References

Blazar et al. 2001. The Journal of Immunology. 166:3174-3183.

Seo et al. 2003. The Journal of Immunology. 171:576-583.

Croft M. 2003. Cytokine and Growth Factor Reviews. 14:265-273.

Vinay et al. 1998. Seminars in Immunology. 10:481-489.

Croft M. 2003. Nature Reviews Immunology. 3:609-620.

Aggarwal B. B. 2003. Nature Reviews Immunology. 3:745-755.

Kwon et al. 2003. Experimental and Molecular Medicine. Vol. 35, No. 1 Pp. 8-16.

Kwon et al. 2000. Molecules and Cells. Vol. 10, No. 2, Pp. 119-126.

TNR9-Splice Variant Structure

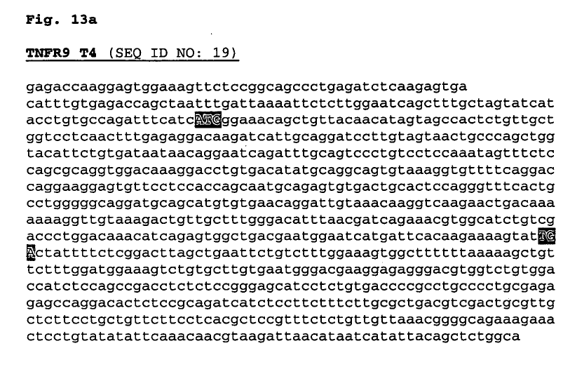

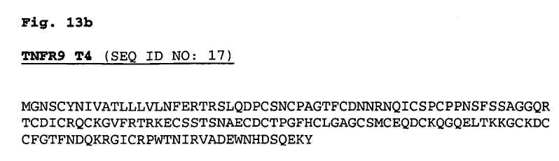

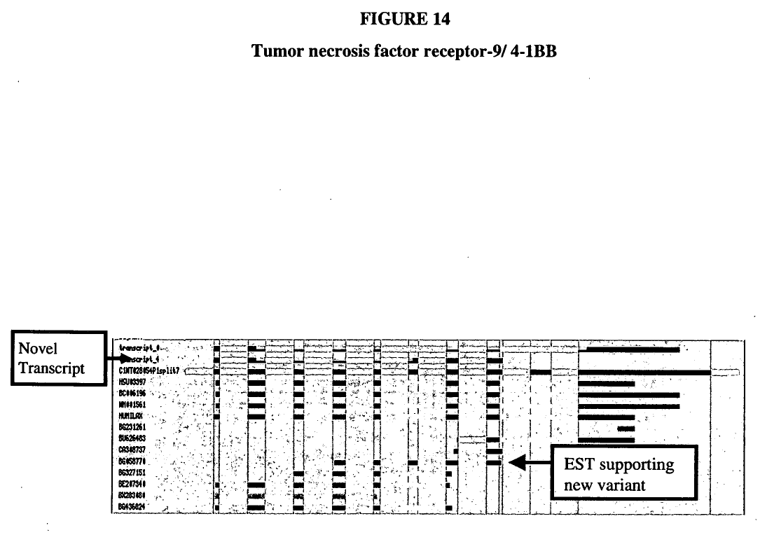

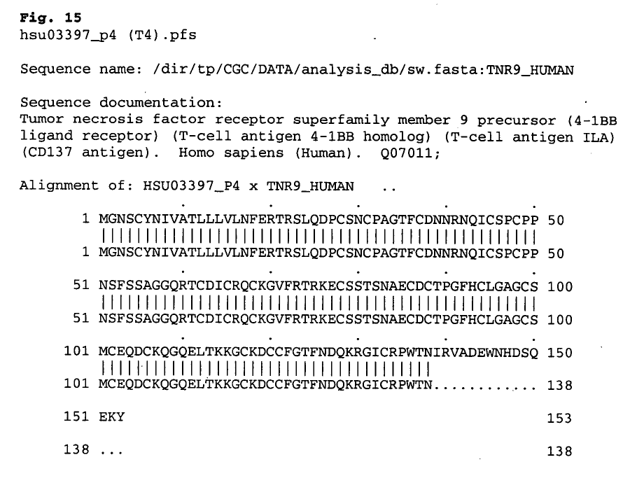

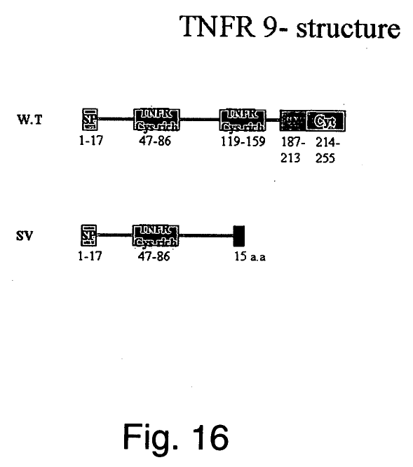

The present inventors uncovered novel splice variants of TNR9 (SEQ ID NOs: 17, 19, FIGS. 13a-b-16) by applying LEADS clustering and assembly algorithm and the annotation process, as described above (Example 1).

This TNR9 (4-1BBR) splice variant results from alternative splicing of the TNR9 gene, thus introducing a new exon 5a (between exons 5 and 6) and a 3′ UTR, leading to an insertion of a stop codon and the generation of a truncated protein. TNR9 splice variant encodes a 153 amino acids long protein which contains the N-terminal signal sequence (residues 1-17), one of the TNFR cys rich repeats (residues 47-86) and a unique sequence of 15 amino acids at the C-terminus of the protein. It is predicated to be a secreted protein due to the fact that it lacks the transmembrane domain.

Therapeutic Applications for the TNR9 Splice Variants of the Present Invention

TNR9 splice variant can serve as a antagonist of TNR9 (4-1-BBR)/4-1BBL interaction. It contains part of the extracellular region of TNR9, thus may be capable of inhibiting TNR9 signaling by competing with the membrane-bound receptor for the ligand-4-1BBL, thus preventing its binding to the cell surface receptor and as a consequence blocking TNR9 activation and its signaling pathway.

Inhibitors of TNR9 signaling pathway, such as this TNR9 splice variant, could have an important therapeutic potential for the treatment of inflammatory diseases such as MS and Crohn's disease, myocardial inflammation, induced by coxackievirus B3, herpetic stromal keratitis (HSK) induced by HSV-1 and to prevent rejection of organ transplant and graft-vs-host disease.

### Example 6

### Interleukin-4 Receptor (IL-4R)

Background

IL-4 is a pleiotropic and multifunctional cytokine produced by activated T cells, mast cells and basophils. IL-4 plays a critical role in regulating the outcome of an immune response by facilitating Th2 cell differentiation and suppressing the differentiation of IFN-γ-producing CD4+ T cells, thereby favoring humoral immune responses. The other important function of IL-4 is the regulation of immunoglobulin class-switching. It induces class-switching to IgE and IgG4 in humanB cells, suggesting a preeminent role of IL-4 in the regulation of allergic conditions. IL-4 also exerts a wide variety of other effects on hematopoietic and nonhematopoietic cells. It enhances the expression of CD23 and class II MHC molecules in B cells and upregulates surface expression of the receptor complex for IL-4. On vascular endothelial cells, IL-4 together with TNF induces the expression of VCAM-1 (vascular cell adhesion molecule 1) and downregulates the expression of E-selectin, thereby changing the adhesive characteristics of endothelial cells and facilitating tissue infiltration by allergic inflammatory cells, such as eosinophils. IL-4 receptors are expressed on hematopoietic cells and a range of nonhematopoietic cells including epithelial, endothelial, muscle, fibroblast and liver cells. On hematopoietic cells, the receptor complex for IL-4 is composed of a 140 kDa high-affinity ligand-binding chain, the IL-4-receptor α chain (IL-4Rα) and the so-called common γ chain (γC) that is shared by IL-2, IL-7, IL-9 and IL-15. In contrast, IL-13Rα1 is the predominant accessory chain of the receptor complex for IL-4 in non-hematopoietic cells. Furthermore, the receptor complex for IL-13 consists of various combinations of the IL-4Rα, IL-13Rα1 and IL-13Rα2. This may explain the redundancy in biological responses mediated by IL-4 and IL-13. Both IL-4 and IL-13 have been implicated in allergic diseases, probably through redundant and independent pathways. Although homodimerized IL-4Rα can generate biological signals within the cell, physiologic signaling requires heterodimerization of IL-4Rα and the accessory chain (γC). Neither IL-4Rα nor γC contains intrinsic kinase activities; rather the IL-4R requires receptor-associated kinases for the initiation of signal transduction. Three members of the Janus kinase (Jak) family- Jak-1, Jak-2 and Jak-3 have been shown to be activated in response to IL-4R engagement and to associate with the components of the receptor complex for IL-4. Jak-1 has been proposed to bind IL-4Rα whereas Jak-3 associates with the γC chain. IL-4-IL-4R engagement results in tyrosine phosphorylation of Jak-1 and Jak-3, leading to tyrosine phosphorylation of IL-4Rα itself, a process that occurs immediately after IL-4R engagement. Five conserved tyrosine residues (Tyr497, Tyr575, Tyr603, Tyr631 and Tyr713) that can potentially be phosphorylated are present in the cytoplasmic domain of IL-4Rα. After tyrosine phosphorylation, these conserved tyrosine residues become potential docking sites for downstream signaling molecules containing Src-homology-domain 2 (SH2) or phosphotyrosine-binding domains.

References

Mueller et al. 2002. Biochimica et Biophysica Acta. 1592:237-250.

Nelms et al. 1999. Annu. Rev. Immunol. 17:701-738.

Pan et al. 1999. Current Opinion in Immunology. 11:615-620.

Gessner et al. 1999/2000. Immunobiology. 201, 285-307.

IL-4R Splice Variants-Structure

The present inventors uncovered novel isoforms of IL-4R as further described hereinbelow, by applying LEADS clustering and assembly algorithm and the annotation process, as described above (Example 1).

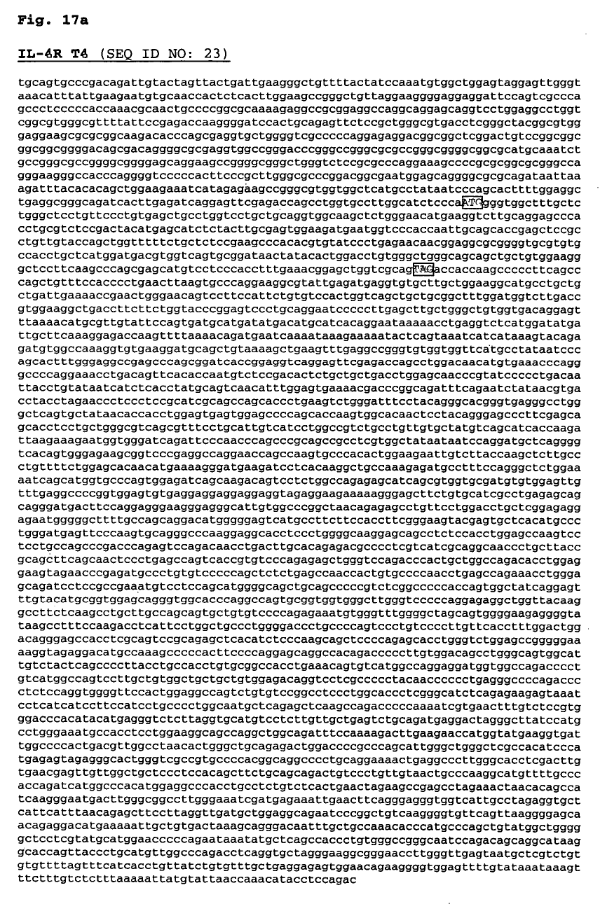

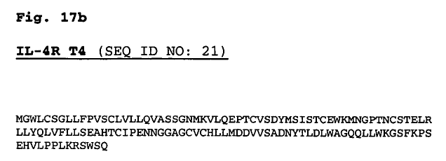

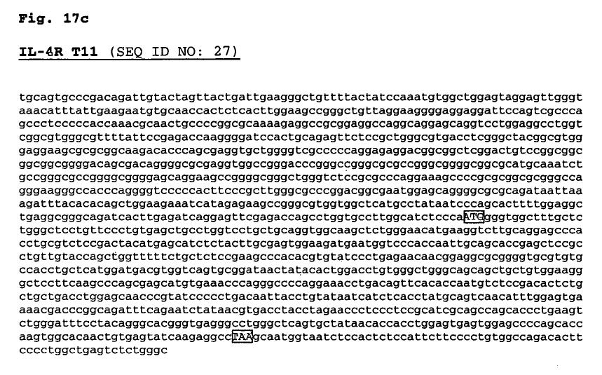

IL-4R splice variant T4 (SEQ ID NO: 21, 23, FIGS. 17a-b-20) results from alternative splicing of the IL-4R gene, introducing a novel exon 4a (between exons 4 and 5), leading to an insertion of a stop codon and the generation of a truncated protein. IL-4R splice variant T4 encodes a 131 amino acids long protein which contains the N-terminal signal sequence (residues 1-25), the complete CRIA domain of IL-4R and a unique sequence of 10 amino acids at the C-terminus of the protein. It is predicated to be a secreted protein due to the fact that it has lost its transmembrane domain.

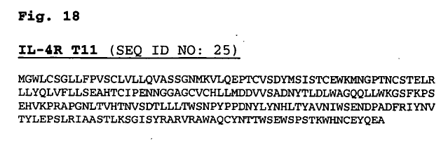

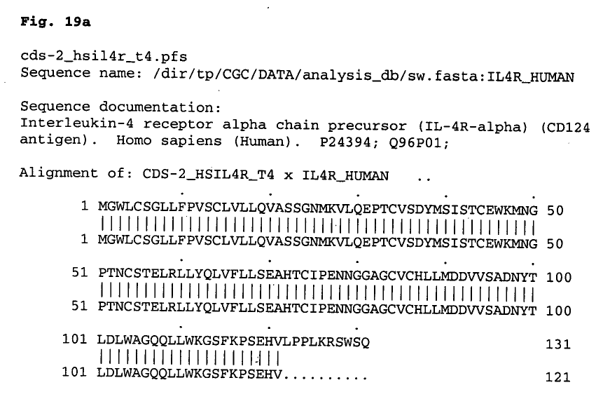

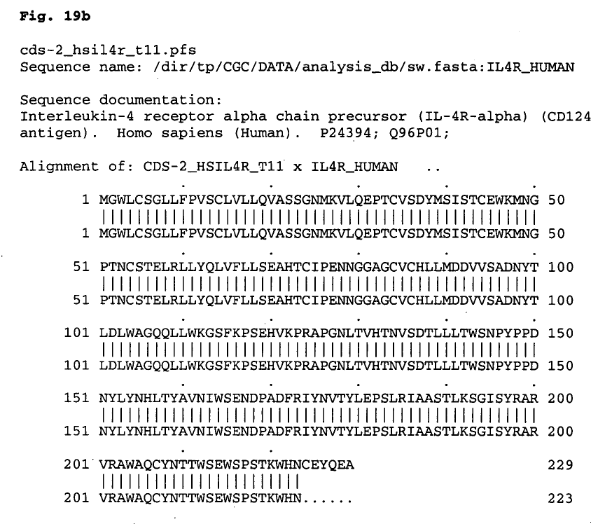

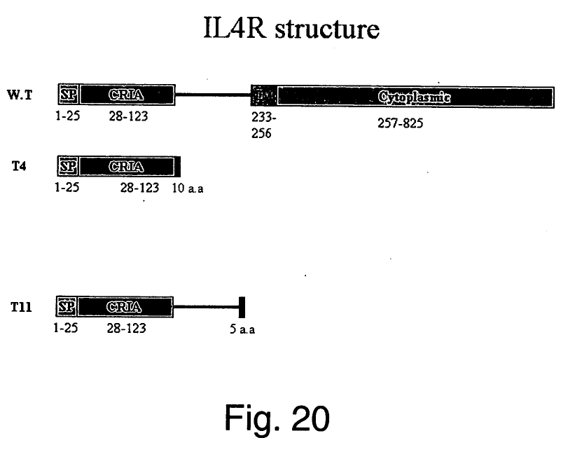

IL-4R splice variant T 11 (SEQ ID NOs: 25, 27, FIGS. 17c, 18-20) results from alternative splicing of the IL-4R gene, thus causing the extension of exon 6 (the last exon before the exon that encodes the transmembrane domain), leading to an insertion of a stop codon and the generation of a truncated IL4R which ends just before the transmembrane domain. IL-4R splice variant T11 encodes a 229 amino acids long protein which contains the N-terminal signal sequence (residues 1-25), the complete CRIA domain of IL-4R and a unique sequence of 6 amino acids at the C-terminus of the protein. It is predicated to be a secreted protein due to the fact that it has lost its transmembrane domain.

Therapeutic Applications for the IL-4R Splice Variants of the Present Invention

Since IL-4Rα is an independent high affinity IL-4 binding subunit, IL-4Rα splice variants, which are secreted forms of the receptor can serve as antagonists of IL-4/IL-4R interaction. They all contain the complete CRIA domain of IL-4Rα while T11 splice variant even contains the whole extracellular region of IL-4Rα and therefore they can inhibit IL-4/IL4R signaling by competing with the membrane-bound receptor for the ligand, thus preventing IL-4R activation. It has been previously reported that the recombinant extracellular domain of IL-4R blocks IL-4 functions in vitro and in vivo, first shown in a murine model of allotransplantation.

IL-4-IL4R signaling pathways play a major role in the pathogenesis of allergic diseases. Moreover, naturally occurring mutations of the IL-4Rα chain have been identified and implicated in a genetic predisposition for atopic asthma. Blocking of IL-4 signaling could therefore have an important therapeutic potential for the treatment of asthma and other allergic disorders. In addition to its role in allergic disorders, IL-4R signaling was shown to be involved in autoimmune diseases and in organ transplantation. Recently, it has been shown that IL-4 may serve multiples roles in the development of lupus. Evidence for a novel role for IL-4 in the development of lupus nephritis comes from recent studies, which suggest that IL-4 may directly promote extracellular matrix deposition in the glomeruli. Blockage of IL-4 signaling may ameliorate glomerulosclerosis and prevent the development of end-stage renal disease and in general might have a therapeutic potential in the treatment of lupus. Thus, in addition to their therapeutic potential in the treatment of asthma and other allergic disorders, IL-4Rα splice variants may be used in the treatment of autoimmunity diseases such as lupus, in organ transplant rejection and graft-vs-host diseases.

### Example 7

### Transforming Growth Factor β Receptor Type II (TGF-β-R/TGR2)

Background

TGF-β belongs to a large family of growth and differentiation factors. It is a potent growth inhibitor of all epithelial and hematopoietic cells and can also induce apoptosis. Three isoforms of TGF-β, designated TGF-β-₁₋₃, with about 70% amino acid sequence identity have been identified in mammals. TGF-β signals through two related transmembrane ser/thr kinase receptors, the type I and type II receptors (TGR1 and TGR2). Signaling is initiated when TGF-β binds to the type II receptor (TGR2, Swissprot Locus No. TGR2_HUMAN) which is followed by recruitment of the type I receptor into a heteromeric complex. Within the complex the type II receptor transphosphorylates and activates the type I receptor kinase which targets downstream signaling components of the pathway. Proteins belong to the SMAD family of intracellular mediators are the only downstream substrates of the type I receptor kinase. Two member of the SMAD family, namely, Smad 2 and Smad 3, are directly phosphorylated by the type I receptor, leading to association of these receptor-regulated SMADs and Smad 4, followed by translocation of the heteromeric complex to the nucleus. In the nucleus, these complexes of SMADs can interact with DNA and with specific DNA binding transcription factors to elicit gene response to TGF-β. In addition to the SMADs, the activated receptor complex can signal through phosphatidylinositol 3-kinase (P13K), protein phosphatase 2A/p70 S6 kinase (PP2A/p70S6K), and various mitogen-activated protein kinase (MAPK) pathways. Since TGF-β inhibits cell growth, escape from the growth inhibition by TGF-β results in uncontrolled cell growth. Mutations in the extracellular domain of the type II receptor were identified in hereditary non-polyposis colon cancer (HNPCC) and in several transformed cells. In addition to defects in the type II receptor, alterations in the type I receptor have been found in prostate, colon and gastric cancer cells, which are insensitive to TGF-β.

Clinical Applications

Although TGF-β can be tumor suppressive, there is increasing evidence that TGF-β secretion by tumor cells and/or stromal cells within the peritumoral microenviroment can contribute to tumor maintenance and progression. The effect of TGF-β is considered to be biphasic: It acts early as a tumor suppressor, probably by inhibiting the proliferation of nontransformed cells and it acts later as tumor promoter, by downregulating cellular adhesion molecules, elevating the expression of metallo-proteases, increasing motility and angiogenesis and causing local and systemic immunosuppression, all of which contribute to tumor progression and metastasis. In support of this view, elevated levels of TGF-β are often observed in advanced carcinomas and have been correlated with diseases progression. The potential tumor promoting effects of TGF-β provide novel molecular targets for interventions. Several approaches have been proposed, including the use of blocking antibodies against TGF-β1, TGF-β2, and TGF-β3, using the extracellular domains of the type II and III TGF-β receptors, which would sequester TGF-β isoforms at tumor sites and prevent binding to cognate receptors and, using adenovirus encoding inhibitors of TGF-β signaling.

References

Dumont et al. 2000. Breast Cancer Res. 2:125-132.

Miyazono K. 1997. International Journal of Hematology. 65:97-104.

Brattain et al. 1996. Current Opinion in Oncology. 8:49-53.

Wrana J. L. 1998. Mineral and Electrolyte Metabolism. 24:120-130.

Moustakas et al. 2001. Journal of Cell Science 114:4359-4369.

Akhurst R. J. 2002. The Journal of Clinical Investigation. Vol. 109, No. 12, Pp. 1533-1536.

Attisano et al. 2002. Science. 296:1646-1647.

Splice Variant Structure

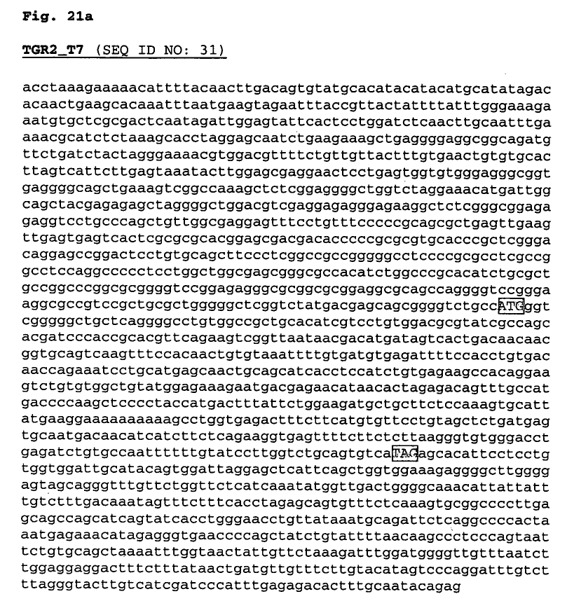

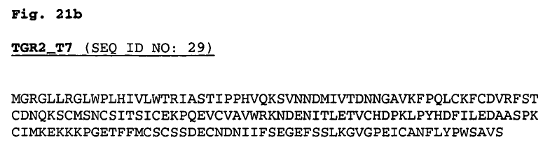

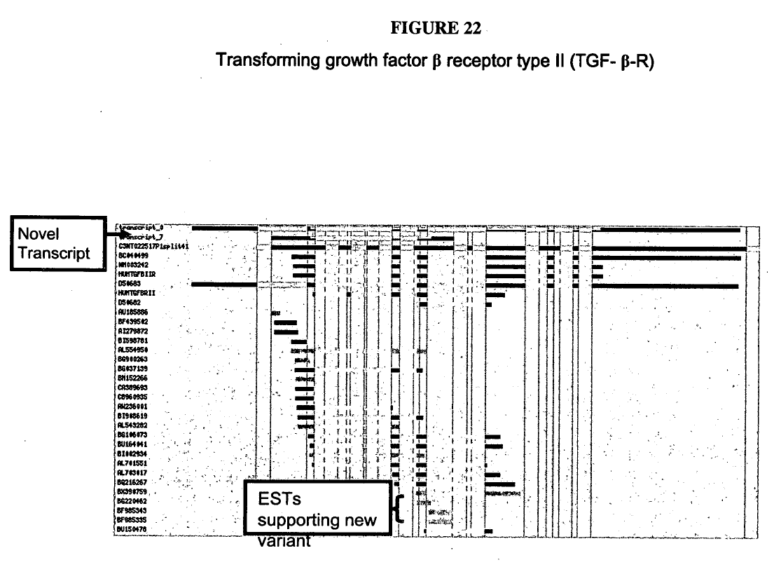

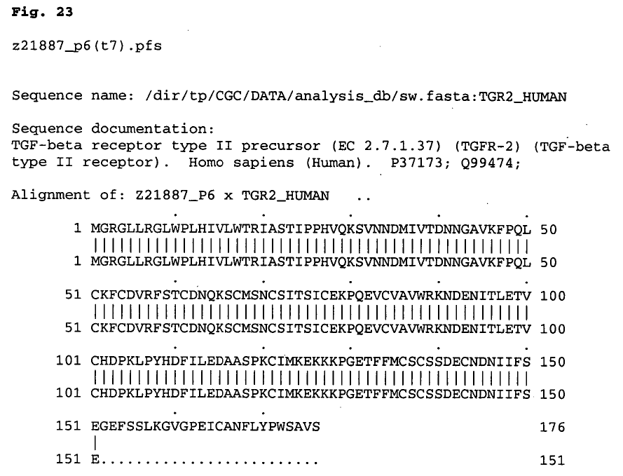

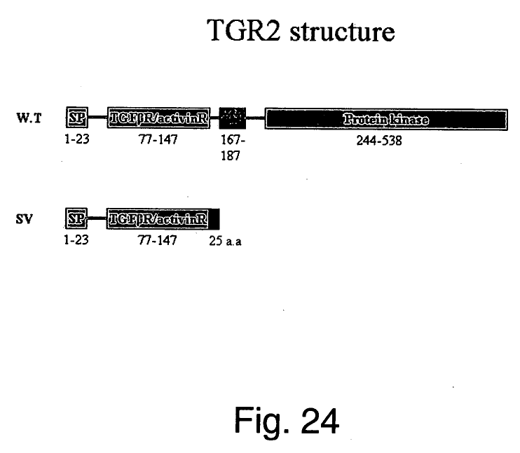

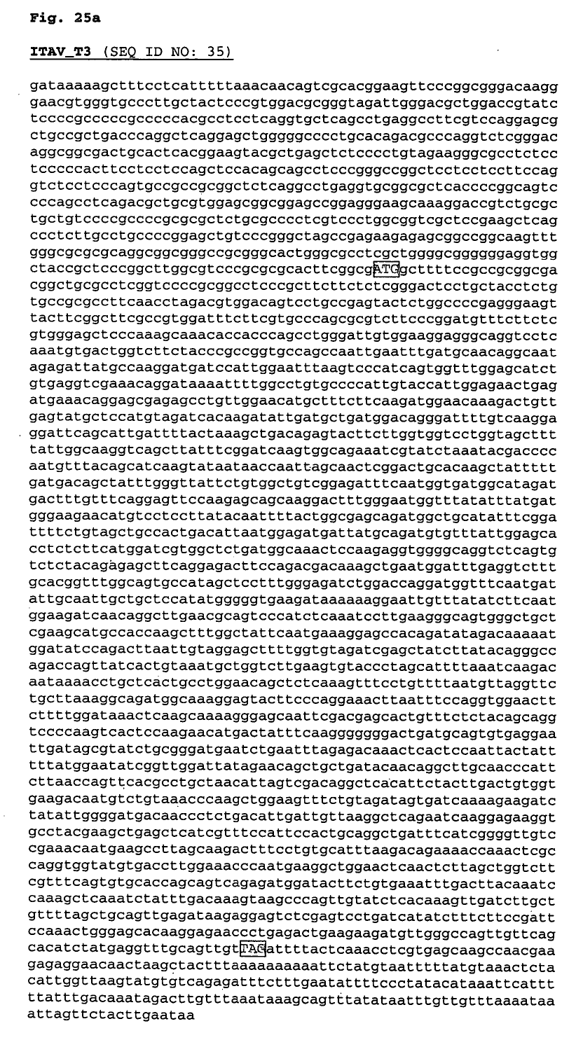

The present inventors uncovered a novel isoform of TGR2 (SEQ ID NOs: 29 and 31, FIGS. 21a-b-24) by applying LEADS clustering and assembly algorithm and the annotation process, as described above (Example 1).

This TGR2 splice variant results from alternative splicing of the TGR2 gene, thus causing the extension of exon 3 (the last exon before the exon that encodes the transmembrane domain) leading to an insertion of a stop codon and the generation of a truncated TGR2 protein which ends just before the transmembrane domain. TGR2 splice variant encodes a 176 amino acids long protein which contains the N-terminal signal sequence (residues 1-23), the complete TGF-βR/ activinR domain and a unique sequence of 25 amino acids at the C-terminus of the protein. It is predicated to be a secreted protein due to the fact that it retains the original N-terminal signal peptide and lacks the transmembrane domain.

Therapeutic Applications for the TGR2 Splice Variant of the Present Invention

Since TGR2 splice variant encodes a soluble receptor which contains the complete TGF-βR/ activinR domain, it is expected to bind TGF-β and thus can inhibit TGF-β/TGR2 signaling by competing with the membrane-bound receptor on the ligand, thus preventing TGF-β from binding to the cell surface receptor and activating it. A soluble form of TGR2 has been previously shown to bind TGF-β and to inhibit its signaling in vitro and in vivo. A chimeric Fc:TGR2 protein was shown to be very attractive because of its high affinity for TGF-β and its effectiveness in a number of animal models. Fc:TGR2 was efficient in reducing tumor metastasis in models of brest cancer and melanoma, whether delivered genetically (transgenic mice) or administered as an injectable circulating drug. Similarly, TGR2 splice variant may be efficient in cancer therapy. In addition to cancer, TGR2 may be used for the treatment of other diseases including organ remodeling diseases/fibrotic diseases, such as chronic renal disease or pulmonary fibrosis, scleroderma and eye's scarring following glaucoma surgery.

### Example 8

### Integrin α-V-ITAV

Background

The integrin family is composed of 15α and 8β subunits that form over twenty different αβ heterodimeric combinations on cell surfaces. Integrins recognize extracellular matrix (ECM) proteins and cell surface immunoglobulin family molecules through short peptide sequences. Several integrins (e.g., αᵥβ₃, α₅β₁, α_{11b}β₃) interact strongly with the tripeptide Arg-Gly-Asp (RGD) sequence within the context of specific ECM or cell surface proteins. While some integrins recognize only a single ECM protein ligand (e.g. α₅β₁ recognizes only fibronectin), others can bind several ligands (e.g., αᵥβ₃ binds vitronectin, fibronectin, osteopontin, fibrinogen, denatured or proteolysed collagen and other matrix proteins). The integrin-mediated adhesion of cells to the ECM leads to bi-directional intracellular signaling events that can regulate cell survival, proliferation and migration. In contrast, inhibition of integrin-ligands interactions suppresses cellular growth or induces apoptotic cell death. Integrin αᵥβ₃, the most promiscuous member of the integrin family, is not widely expressed in normal tissue. It is not generally expressed on ephithelial cells and is expressed only at low levels on resting vascular and uterine smooth muscle as well as endothelium and on certain activated leukocytes, macrophages and osteoclasts. Although αᵥβ₃ is not highly expressed on normal cells, it is expressed on tumor cells including late-stage glioblastomas, ovarian carcinoma and melanomas. It contributes to the progression of melanoma by regulating melanoma cell proliferation, survival and metastases. The αᵥβ₃ on endothelium cells can take part in the angiogenic process in several ways. It regulates cell adhesion to the matrix, transmit signals to the nucleus of the cell and is pro-angiogenic by co-operating with VEGFR-2, a pro-angiogenic receptor through the activation of cell signalling and the regulation of cell cycle gene expression. The two β chains identified with αᵥ and angiogenesis are β₃ and β₅. These subunits share 53% homology, however their ligand specificities are different. αᵥβ₃ prefers to bind to osteopontin while αᵥβ₅ prefers vitronectin. Two different cytokine-dependent pathways participate in the activation of these two integrins. The αᵥβ₃ pathway involves basic FGF (FGF-2) or TNF-α, whereas αᵥβ₅ uses VEGF, TGF-α, or PMA. Since the integrin αᵥ subunit is widely expressed on most cell types and associates with several different β subunits, the expression of αᵥβ₃ is likely to be regulated by β transcription. There are additional αᵥ integrin complexes associated with angiogenesis and blood vessels. They include αᵥβ₁ associated with brain blood vessels and with squamous cell carcinoma cell migration; αᵥβ₈, identified on tumor cells; and αᵥβ₆ which induces secretion of MMP-2 in colon cancer and is important in the progression of this disease.

Clinical Applications

The role of integrins in pathological processes in both acute and chronic diseases include ocular, cancer (primary tumors and metastasis), cardiovascular (stroke and heart failure) and inflammatory conditions (rheumatoid arthritis). The αᵥ integrin has been found to be associated with multiple tumor types, including melanoma, breast, renal, cervical, colon, prostate, bladder, and lung carcinoma. Antibodies to αᵥ prevent human melanoma tumor formation in nude mice and antagonists of αᵥβ₃ potentially inhibit angiogenesis in a number of animal models. Thus, blocking the αᵥ integrin serves as an important therapeutic strategy in cancer therapy. Inhibitors of integrin function include blocking monoclonal antibody and peptide antagonist, which mimics the RGD ligand recognition domain common to αᵥ integrin ligands, are in phase II clinical trials.

References

Kerr et al. 2000. Exp.Opin. Invest. Drugs 9(6):1271-1279.

Tucker G. C. 2003. Current Opinion in Investigational Drugs. 4(6):722-731.

Mould et al. 2000. The Journal of Biological Chemistry. Vol. 275, No. 27, Pp. 20324-20336.

ITAV-Splice Variant Structure

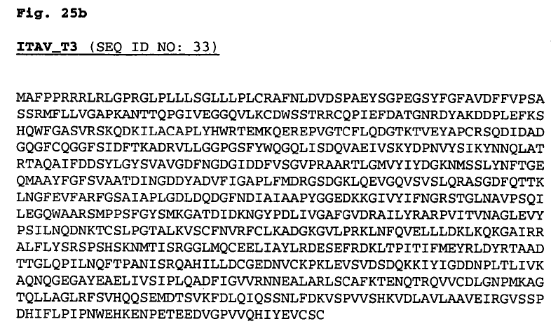

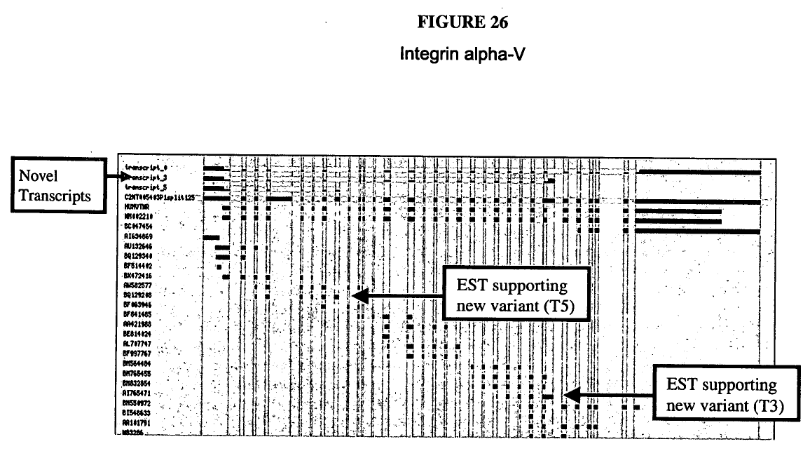

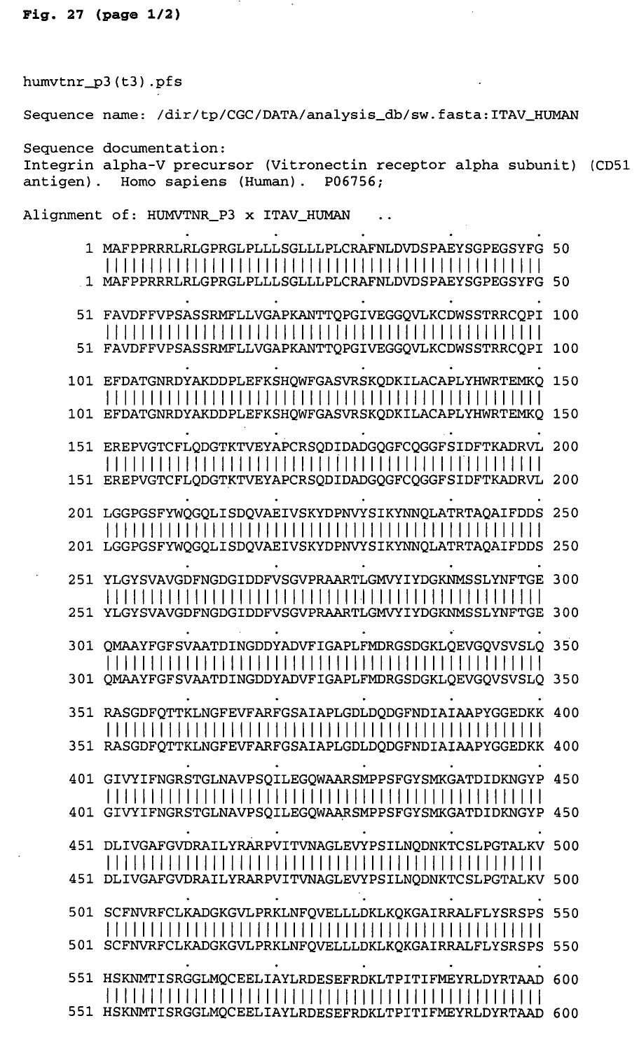

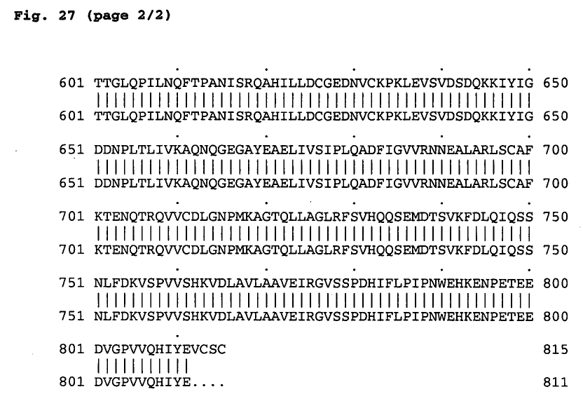

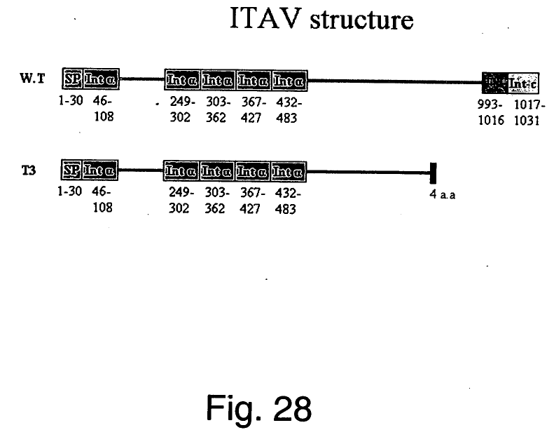

The present inventors uncovered a novel isoform of ITAV (SEQ ID NOs: 33 and 35, FIGS. 25a-b-28) by applying LEADS clustering and assembly algorithm and the annotation process, as described above (Example 1).

The ITAV splice variant T3 results from alternative splicing of the ITAV gene, causing an extension of exon 24, leading to the insertion of a stop codon and the generation of a truncated ITAV protein which ends before the transmembrane domain. ITAV splice variant T3 has an ORF of 815 amino acids: 811 amino acids of the wild-type protein and a unique sequence of 4 amino acids at the C-terminus. It contains most of the extracellular region of ITAV (811 amino acids out of 993 of the wild-type), including the five integrin alpha repeats. It is predicated to be a secreted protein due to the fact that it retains the original N-terminal signal peptide (amino acids 1-30) and lacks the transmembrane domain (amino acids 993-1016 of the wild-type).

Therapeutic Applications for the ITAV Splice Variant of the Present Invention

ITAV splice variant T3 can serve as an antagonist of a variety of integrin interactions. It contains most of the extracellular region of ITAV and therefore is likely to bind the ligands. This splice variant can inhibit integrin signaling by competing with the membrane-bound receptor for the different ligands, thus preventing their binding to the cell surface receptor and as a consequence block integrin activation and signaling pathway. Alternatively, it can compete with the wild-type membrane ITAV for binding of the β subunit, thus preventing the heterodimerization of αᵥ with the β subunit and the subsequent signaling.

Because of the overwhelming evidence favoring the role of αᵥ integrin in the pathogenesis of a wide array of diseases as cancer, cardiovascular and inflammation, inhibitors of this molecule, such as the ITAV splice variant of the present invention, may have an important therapeutic potential. ITAV splice variant can play a critical role in the treatment of the following pathological conditions: cancer (in general, but in particular colon and melanoma); cardiovascular diseases, such as atherosclerosis, restenosis, ischemia and reperfusion injury; immunological related diseases such as immunodeficiency, allergies, asthma, psoriasis, RA and inflammatory bowl diseases/chrone's disease; metabolism related diseases, such as diabetes and diabetes related retinopathy; osteoporosis, sepsis and wound healing.

### Example 9

### Interleukin-10 Receptor β Chain

Background

IL-10 was first described as a cytokine that is produced by T helper 2 (Th2) cells that inhibits interferon (IFN)-γ synthesis in Th1 cells. It is a homodimer, produced mainly by macrophages, which has a crucial role in immunoregulation. Its expression is regulated by several endogenous and exogenous factors such as endotoxin, tumor necrosis factor (TNF)-α, catecholamines, and cAMP-elevating agents. IL-10 activity is mediated by its specific cell surface receptor complex, which is expressed on a variety of cells, in particular immune cells. The IL-10 receptor is composed of two different chains, α and β (CRFB4), both members of the class II cytokine receptor family. These receptors are transmembrane glycoproteins whose extracellular domains consist of about 210 amino acids comprising two tandem fibronectin type III domains and having several conserved amino acid important for the secondary structure. The interaction of IL-10R with IL-10 seems to be highly complex. The IL-10Rβ chain (Swissprot Locus No. I10S_HUMAN) is essential for IL-10-mediated effects as CRFB4-deficient mice display the same phenotype as IL-10 deficient mice.

Interestingly, in cells which express IL-10Rβ exclusively, no IL-10/IL-10R complexes are formed, suggesting that only IL-10/IL-10Rα complexes interact with the β-chain. However in cells expressing both the IL-10Rα and β chains the characteristic STAT transcription factor activation pattern for IL-10 signaling is observed. The IL-10/IL-10R interaction activates the tyrosine kinases Jak1 and Tyk2, which are associated with the IL-10Rα and IL-10Rβ2, respectively. The receptor engagement and tyrosine phosphorylation activates the cytoplasmically localized inactive transcription factors STAT 1, 3, and 5, resulting in their translocation to the nucleus and downstream gene activation. IL-10 signaling results in the inhibition of immune functions. It controls inflammatory processes by suppressing the expression of proinflammatory cytokines, chemokines, adhesion molecules, as well as antigen-presenting and costimulatory molecules in monocytes/macrophages, neutrophils, and T cells. As all of these inflammatory proteins are transcriptionally controlled by NF-κB it wasn't surprising to find out that IL-10 exerts a significant part of its anti-inflammatory properties by inhibiting this transcription factor. Antigen-presenting cells and lymphocytes are the primary targets of IL-10. Direct effects on these populations explains the major immunological impact of this cytokine, including the regulation of the Th1/Th2 balance. IL-10 reverses the Th1 cytokine pattern present. It promotes the development of a type 2 cytokine pattern by inhibiting the IFN-γ production of T lymphocytes particularly via the suppression of IL-12 synthesis in accessory cells. According to this, IL-10 costimulates the proliferation and differentiation of B cells, which is important in the adequate defense against intestinal parasites, neutralization of bacterial toxins, and in local mucosa defense. Moreover, IL-10 suppresses the production of proinflammatory cytokines as IL-1β, IL-6, IL-8, G-CSF, GM-CSF, TNFα while enhances the production of anti-inflammatory mediators such as IL-IRA and soluble TNFα receptors. In addition it inhibits the capacity of monocytes/macrophages and dendritic cells to present antigen to T cells. This is realized by down-regulation of cell surface levels of MHC class II, of costimulatory molecules such as CD86 and of some adhesion molecules such as CD58.

Clinical Applications

Its considerable anti-inflammatory effects and ability to act as a main suppressor of cellular immunity raises the question of the IL-10 expression under pathophysiological conditions. Both overexpression (e.g., in lymphoma, melanoma, carcinoma) as well as IL-10 deficency were found (e.g., in inflammatory bowel disease, psoriasis) and seems to have a pathophysiological significance. IL-10 overexpression in different malignancies might contribute to tumor development, in particular, by suppressing the antitumor immune response. Moreover, IL-10 might even be a tumor cell growth factor in certain tumors such as B cell lymphoma and melanoma. In contrast to several malignancies, where there is an overexpression of IL-10, a relative deficiency is considered to be of pathophysiological relevance in chronic inflammatory disorders characterized by the predominance of a type1 cytokine pattern. These included psoriasis, inflammatory bowel disease such as Crohn's diseases, multiple sclerosis, rheumatoid arthritis, transplant rejection, and allergic contact dermatitis. The immunomodulatory properties of IL-10 and the promising results from IL-10 delivery on the course of several inflammatory diseases in experimental models induced the interest on clinical application of IL-10. So far human recombined IL-10 (ilodecakin/Tenovil; Schering-Plough Research, Kenilworth, N.J.) has been tested in healthy volunteers, patients with Crohn's disease, rheumatoid arthritis, psoriasis, hepatitis C infection, HIV infection, and for the inhibition of therapy associated cytokine releases in organ transplantation and Jarisch-Herxheimer reaction. Application of IL-10 in humans seems to be safe and immunologically active. The clinical effects of recombinant IL-10, however, have been quite heterogeneous in different entities. Whereas almost no effect was seen in rheumatoid arthritis and CD, significant response was observed in psoriasis.

References

Asadullah et al. 2003. Pharmacol Rev. 55:241-269.

Kotenko S. V. 2002. Cytokine & Growth Factor Reviews. 13:223-240.

Walter M. R. 2002. Immunologic Research. 26/1-3:303-308.

Moore et al. 2001. Annu. Rev. Immunol. 19:683-765.

IL-10-Rβ-Splice Variant Structure

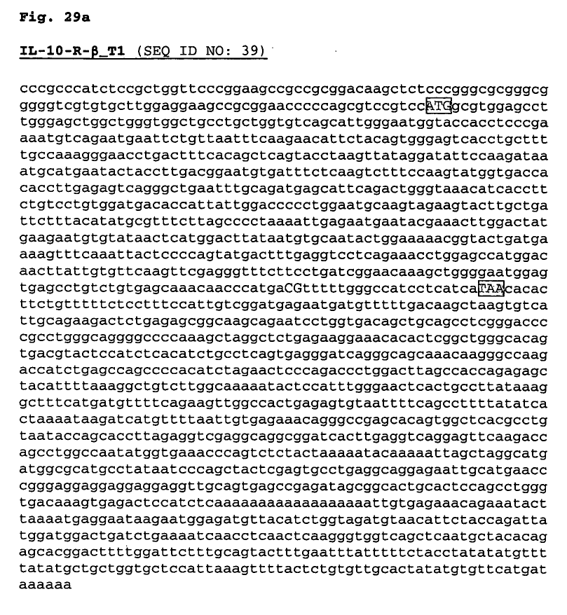

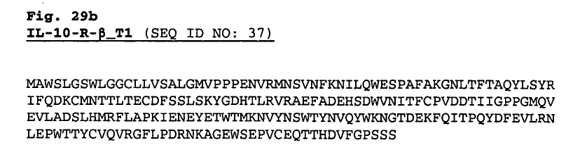

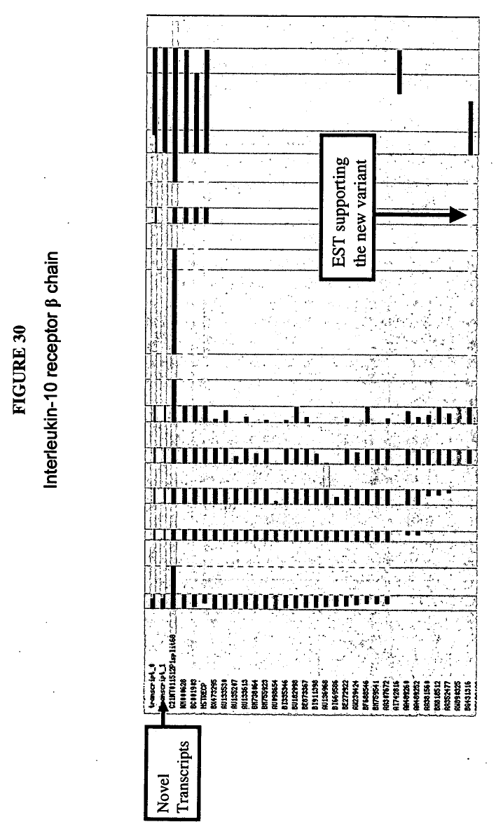

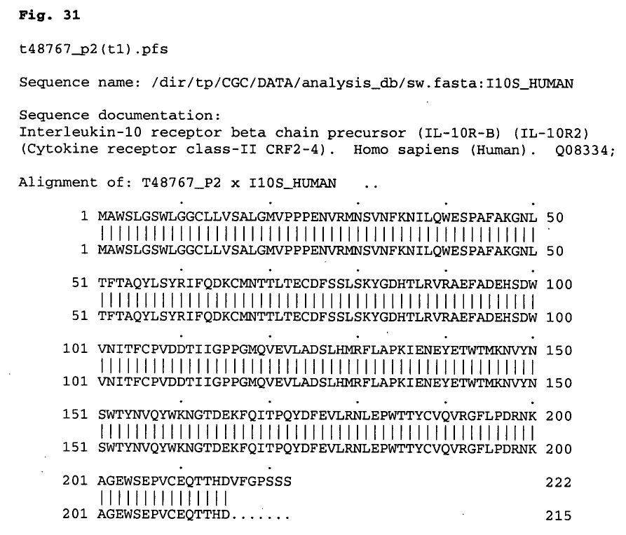

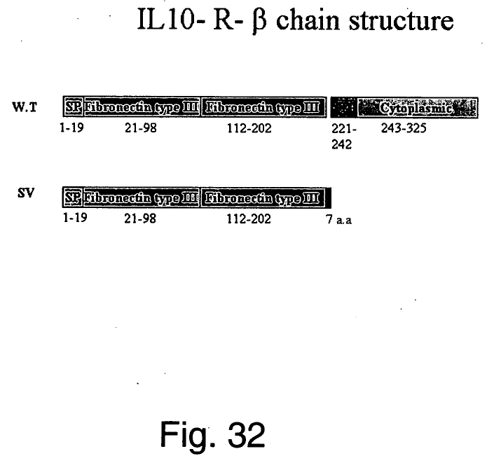

The present inventors uncovered a novel isoform of IL-10Rβ (SEQ ID Nos. 37 and 39, FIGS. 29a-b-32). IL-10-Rβ splice variant results from alternative splicing of the IL-10-Rβ gene, thus causing the skipping of exon 6 (the exon which encodes the transmembrane domain), leading to the insertion of a stop codon and the generation of a truncated protein. IL-10-Rβ splice variant encodes a 222 amino acids long protein which contains the N-terminal signal sequence (residues 1-17), the complete 2 fibronectin type III domains and a unique sequence of 7 amino acids at the C-terminus of the protein. It is predicated to be a secreted protein due to the fact that it has lost its transmembrane domain.

Therapeutic Applications for the IL-10-Rβ Splice Variant of the Present Invention

IL-10 exhibits low in vivo half life. Therefore extension its half life has a therapeutic advantage. Soluble receptors have been shown to exhibit agonistic properties including, increasing the molecular internal stability of the ligand, protection from proteolysis and modification of the pharmacokinetic properties of the ligand, namely, increasing its in vivo half-life while decreasing its clearance. IL-10-Rβ splice variant which encodes a soluble receptor might serve as an agonist, increaseing IL-10 half-life in vivo and therefore enhancing its biological effect. Thus, this splice variant may have an important therapeutic potential for the treatment of the following pathological conditions: inflammatory diseases, such as, psoriasis, inflammatory bowel diseases (Crohn's disease), colitis ulcerative, multiple sclerosis, RA, transplant rejection and allergic contact dermatitis; hepatitis C infection; HIV infection and atherosclerosis.

### Example 10

### Interferon-α/β-Receptor-1-IFNAR1-INR1

Background

Type I interferons (IFNs), initially identified for their ability to protect cells from viral infections, are truly pleiotropic cytokines. They are also implicated in both normal and neoplastic cell growth regulation and in modulating both innate and adaptive immune responses to microbial challenge. All type I IFNs, IFN-αs, IFN-β, IFN-ω,

IFN-κ, and IFN-τ, are functionally active as monomers and activate a specific receptor complex composed of two major subunits, IFNAR-1/INR1 and IFNAR-2/INR2. The high affinity interaction between IFN-α/β and its specific cell surface receptor leads to receptor aggregation and the activation of receptor-associated cytoplasmic tyrosine kinases of the Jak family- Jak1 and Tyk2. These in turn phosphorylate intracellular tyrosine residues of the IFNAR-1 and IFNAR-2 chains, that serve as recruitment sites for the signal transducers and activators of transcription (STAT) proteins, Stat 1-5. Once associated with the activated receptor, the STAT become phosphorylated, then form both homodimers and heterodimers, which translocate to the nucleus and bind specific DNA sequences within the promoter regions of IFN-sensitive genes (ISG). The Jak-Stat pathway is an essential signaling pathway for the transcription of many ISGs, whose protein products mediate specific IFN-dependent biologic responses. IFNs mediate a critical role in innate cellular defense against viral infection. Mice deficient in IFN-β or in IFNAR-1 are highly susceptible to viral infections. The antiviral activity of INFs include inhibition of viral replication and protein synthesis and the induction of viral mRNA degradation. In addition to their antiviral activity, IFNs exhibit growth inhibitory activity, either by mediating cell death (through caspases) or by modulating the expression of proteins regulating cell cycle entry and exit, hence mediating growth arrest. IFNs are also involved in the regulation of immune response towards viral or tumor challenge. A well-characterized function of IFNs is their ability to upregulate MHC class I expression and consequently promote CD8+T cell responses. Moreover, IFNs can regulate the expression of key cytokines that influence T cell responses, namely, IL-12, IL-15 and IFN-γ and of CC— chemokines. IFNs-α/β regulate the functions of immune cells from different lineages including NK cells, dentritic cells and B/T lymphocytes.

Clinical Application

Due to their growth inhibitory activity and the modulation of immune responses, type I interferons have been used as therapeutic agents against a variety of solid tumors and hematological malignancies. IFN-α has been approved for the treatment of chronic myelogenous leukemia (CML), multiple myeloma and hairy cell leukemia.

At this time, IFN-α is the treatment of choice for CML patients not eligible for allogeneic bone marrow transplantation. In addition, it may have the potential therapeutic value in the treatment of several lymphomas. Apart from the widespread therapeutic indications for IFNs in the treatment of neoplasias, their efficacy as therapeutic agents for the treatment of viral infections and autoimmune diseases has been proved. IFN-α is the treatment of choice for hepatitis B and C infections and accumulating evidence supports the use of IFN-β for the treatment of multiple sclerosis.

References

Deonarain et al. 2002. Current Pharmaceutical Design. Vol. 8, No. 24, Pp. 2131-2137.

Brierley et al. 2002. Journal of Interferon and Cytokine Research. 22:835-845.

INR1-Splice Variant Structure

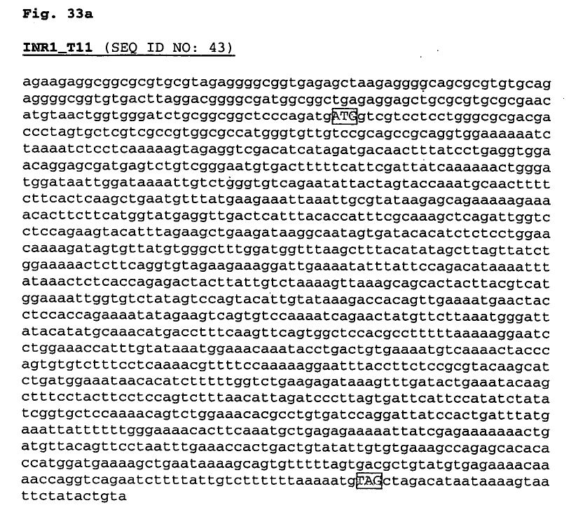

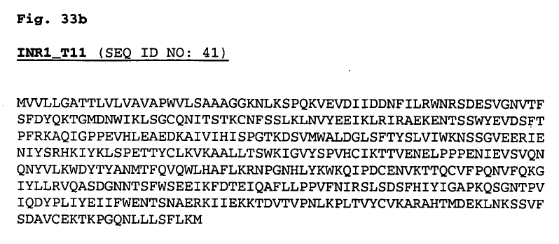

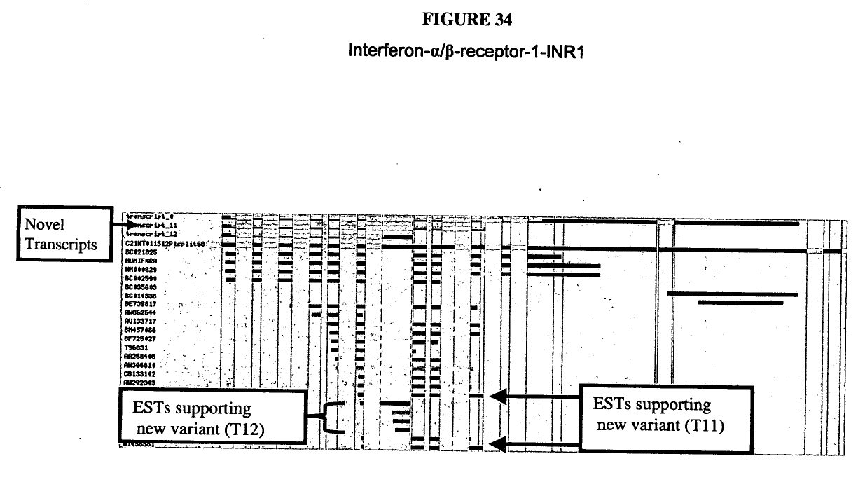

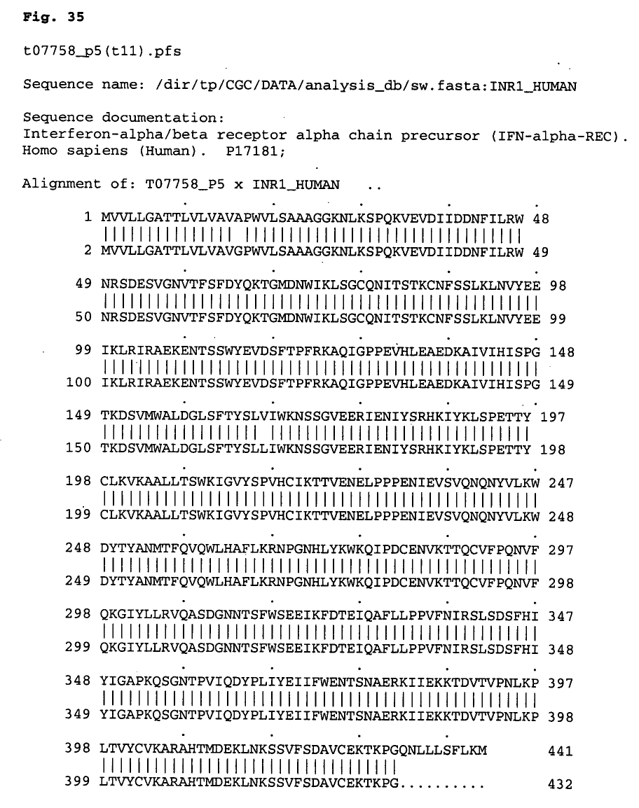

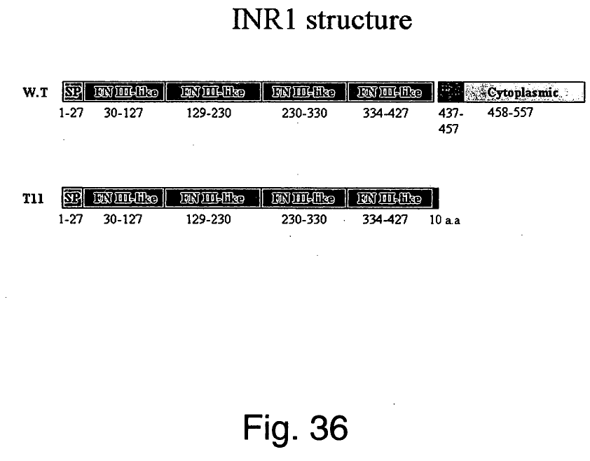

The present inventors uncovered a novel isoform of INR1 (SEQ ID NOs: 41 and 43, FIGS. 33a-b-35). INR1 splice variant 11 results from alternative splicing of the INR1 gene, thus causing an extension of exon 9, leading to an insertion of a stop codon and the generation of a truncated protein. INR1 splice variant T 11 encodes a 441 amino acids long protein which contains the N-terminal signal sequence (residues 1-27), the complete extracellular portion of the wild-type INR1 (up to amino acid 427), including the four fibronectin type III-like domains and a unique sequence of 10 amino acids at the C-terminus of the protein. It is predicted to be a secreted protein since it does not contain the transmembrane domain (residues 437-457).

Therapeutic Applications for the INR1 Splice Variant of the Present Invention

Although the activity and specificity of function make the IFNs potentially therapeutic agents, they are not ideal drugs, exhibiting low stability in vivo. Thus, there is an intense interest in developing alternative or improved molecules that demonstrate IFNs function but have superior pharmacological properties. For example, PEGylation of type I IFNs extends the serum half-life and duration of therapeutic activity. PEGylation of IFN-α and IFN-β increased their serum half-life by 6 and 5 fold, respectively, however the PEGylated form of IFN-β exhibited less efficient systemic distribution with some evidence of induction of neutralizing antibodies. As opposed to their well-characterized function as competitive inhibitors (antagonists), soluble receptors have been shown to exhibit agonistic properties. These include increasing the molecular internal stability of the ligand, protection from proteolysis and modification of the pharmacokinetic properties of the ligand, namely, increasing its in vivo half-life while decreasing its clearance.

INR1 splice variants which encode soluble receptors might serve as agonists, increaseing IFNs half-life in vivo and therefore enhancing their biological effect. Thus, this splice variant may have an important therapeutic potential for the treatment of the following pathological conditions: cancer, such as, solid tumors (e.g., glioblastoma, renal cell carcinoma, melanoma) and hematological malignancies (e.g., chronic myelogenous leukemia (CML), multiple myeloma, non-Hodgkin's lymphomaand hairy cell leukemia), viral infections (e.g., hepatitis B/C, herpes and human papilloma virus) and autoimmune diseases such as multiple sclerosis.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An isolated polynucleotide comprising a nucleic acid sequence encoding a polypeptide having an amino acid sequence at least 70% identical to SEQ ID NO: 1, as determined using the LALIGN software of EMBnet Switzerland (http://www.ch.embnet.org/index.html) using default parameters.

2. The isolated polynucleotide of claim 1, wherein said nucleic acid sequence is as set forth in SEQ ID NO: 3.

3. The isolated polynucleotide of claim 1, wherein said nucleic acid sequence is as set forth in SEQ ID NO: 4.

4. The isolated polynucleotide of claim 1, wherein said polypeptide is as set forth in SEQ ID NO: 1.

5. The isolated polynucleotide of claim 1, wherein said polypeptide is as set forth in SEQ ID NO: 2.

6. An isolated polynucleotide as set forth in SEQ ID NO: 4.

7. An isolated polynucleotide as set forth in SEQ ID NO: 3.

8. An isolated polypeptide as set forth in SEQ ID NO: 1.

9. An isolated polypeptide as set forth in SEQ ID NO: 2.

10. A nucleic acid construct comprising the isolated polynucleotide of claim 1.

11. The nucleic acid construct of claim 10, further comprising a promoter for regulating transcription of the isolated polynucleotide in sense or antisense orientation.

12. The nucleic acid construct of claim 10, further comprising a positive and a negative selection markers for selecting for homologous recombination events.

13. A host cell comprising the nucleic acid construct of claim 10.

14. An isolated polypeptide comprising an amino acid sequence at least 70% identical to SEQ ID NO: 1, as determined using the LALIGN software of EMBnet Switzerland (http://www.ch.embnet.org/index.html) using default parameters or an active portion thereof.

15. An antibody or an antibody fragment being capable of specifically binding a polypeptide having an amino acid sequence at least 70% identical to SEQ ID NO: 1, as determined using the LALIGN software of EMBnet Switzerland (http://www.ch.embnet.org/index.html) using default parameters.

16. An oligonucleotide specifically hybridizable with a nucleic acid sequence encoding a polypeptide having an amino acid at least 70% identical to SEQ ID NO: 1, as determined using the LALIGN software of EMBnet Switzerland (http://www.ch.embnet.org/index.html) using default parameters.

17. A pharmaceutical composition comprising a therapeutically effective amount of a polypeptide having an amino acid sequence at least 70% identical to SEQ ID NO: 1, as determined using the LALIGN software of EMBnet Switzerland (http://www.ch.embnet.org/index.html) using default parameters and a pharmaceutically acceptable carrier or diluent.

18. A method of treating Met-related disease in a subject, the method comprising upregulating in the subject expression of a polypeptide having an amino acid sequence at least 70% identical to SEQ ID NO: 1 as determined using the LALIGN software of EMBnet Switzerland (http://www.ch.embnet.org/index.html) using default parameters, thereby treating the Met-related disease in a subject.

19. The method of claim 18, wherein said upregulating expression of said polypeptide is effected by:
(i) administering said polypeptide to the subject; and/or
(ii) administering an expressible polynucleotide encoding said polypeptide to the subject.

20. An isolated polynucleotide comprising a nucleic acid sequence encoding a polypeptide having an amino acid sequence at least 75% identical to SEQ ID NO: 5, as determined using the LALIGN software of EMBnet Switzerland (http://www.ch.embnet.org/index.html) using default parameters.

21. The isolated polynucleotide of claim 20, wherein said nucleic acid sequence is as set forth in SEQ ID NO: 8.

22. The isolated polynucleotide of claim 20, wherein said nucleic acid sequence is as set forth in SEQ ID NO: 7.

23. The isolated polynucleotide of claim 20, wherein said polypeptide is as set forth in SEQ ID NO: 5.

24. The isolated polynucleotide of claim 20, wherein said polypeptide is as set forth in SEQ ID NO: 6.

25. An isolated polynucleotide as set forth in SEQ ID NO: 8.

26. An isolated polynucleotide as set forth in SEQ ID NO: 7.

27. An isolated polypeptide as set forth in SEQ ID NO: 5.

28. An isolated polypeptide as set forth in SEQ ID NO: 6.

29. A nucleic acid construct comprising the isolated polynucleotide of claim 20.

30. The nucleic acid construct of claim 29, further comprising a promoter for regulating transcription of the isolated polynucleotide in sense or antisense orientation.

31. The nucleic acid construct of claim 29, further comprising a positive and a negative selection markers for selecting for homologous recombination events.

32. A host cell comprising the nucleic acid construct of claim 29.

33. An isolated polypeptide comprising an amino acid sequence at least 75% identical to SEQ ID NO: 5, as determined using the LALIGN software of EMBnet Switzerland (http://www.ch.embnet.org/index.html) using default parameters or an active portion thereof.

34. An antibody or an antibody fragment being capable of binding a polypeptide having an amino acid sequence at least 75% identical to SEQ ID NO: 5, as determined using the LALIGN software of EMBnet Switzerland (http://www.ch.embnet.org/index.html) using default parameters.

35. An oligonucleotide hybridizable with a nucleic acid sequence encoding a polypeptide having an amino acid at least 75% identical to SEQ ID NO: 5, as determined using the LALIGN software of EMBnet Switzerland (http://www.ch.embnet.org/index.html) using default parameters.

36. A pharmaceutical composition comprising a therapeutically effective amount of a polypeptide having an amino acid sequence at least 75% identical to SEQ ID NO: 5, as determined using the LALIGN software of EMBnet Switzerland (http://www.ch.embnet.org/index.html) using default parameters and a pharmaceutically acceptable carrier or diluent.

37. A method of treating an IL-6-related disease in a subject, the method comprising upregulating in the subject expression of a polypeptide having an amino acid sequence at least 75% identical to SEQ ID NO: 5 as determined using the LALIGN software of EMBnet Switzerland (http://www.ch.embnet.org/index.html) using default parameters, thereby treating the IL-6-related disease in the subject.

38. The Method of claim 37, wherein said upregulating expression of said polypeptide is effected by:
(i) administering said polypeptide to the subject; and/or
(ii) administering an expressible polynucleotide encoding said polypeptide to the subject.

39. An isolated polynucleotide comprising a nucleic acid sequence encoding a polypeptide having an amino acid sequence at least 85% identical to SEQ ID NO: 9, as determined using the LALIGN software of EMBnet Switzerland (http://www.ch.embnet.org/index.html) using default parameters.

40. The isolated polynucleotide of claim 39, wherein said nucleic acid sequence is as set forth in SEQ ID NO: 11.

41. The isolated polynucleotide of claim 39, wherein said nucleic acid sequence is as set forth in SEQ ID NO: 12.

42. The isolated polynucleotide of claim 39, wherein said polypeptide is as set forth in SEQ ID NO: 9.

43. The isolated polynucleotide of claim 39, wherein said polypeptide is as set forth in SEQ ID NO: 10.

44. An isolated polynucleotide as set forth in SEQ ID NO: 11.

45. An isolated polynucleotide as set forth in SEQ ID NO: 12.

46. An isolated polypeptide as set forth in SEQ ID NO: 10.

47. An isolated polypeptide as set forth in SEQ ID NO: 9.

48. A nucleic acid construct comprising the isolated polynucleotide of claim 39.

49. The nucleic acid construct of claim 48, further comprising a promoter for regulating transcription of the isolated polynucleotide in sense or antisense orientation.

50. The nucleic acid construct of claim 48, further comprising a positive and a negative selection markers for selecting for homologous recombination events.

51. A host cell comprising the nucleic acid construct of claim 48.

52. An isolated polypeptide comprising an amino acid sequence at least 85% identical to SEQ ID NO: 9, as determined using the LALIGN software of EMBnet Switzerland (http://www.ch.embnet.org/index.html) using default parameters or an active portion thereof.

53. An antibody or an antibody fragment being capable of binding a polypeptide having an amino acid sequence at least 85% identical to SEQ ID NO: 9, as determined using the LALIGN software of EMBnet Switzerland (http://www.ch.embnet.org/index.html) using default parameters.

54. An oligonucleotide hybridizable with a nucleic acid sequence encoding a polypeptide having an amino acid at least 85% identical to SEQ ID NO: 9, as determined using the LALIGN software of EMBnet Switzerland (http://www.ch.embnet.org/index.html) using default parameters.

55. A pharmaceutical composition comprising a therapeutically effective amount of a IL-7 polypeptide having an amino acid sequence at least 85% identical to SEQ ID NO: 9, as determined using the LALIGN software of EMBnet Switzerland (http://www.ch.embnet.org/index.html) using default parameters and a pharmaceutically acceptable carrier or diluent.

56. A method of treating IL-7-related disease in a subject, the method comprising upregulating in the subject expression of a polypeptide having an amino acid sequence at least 85% identical to SEQ ID NO: 9 as determined using the LALIGN software of EMBnet Switzerland (http://www.ch.embnet.org/index.html) using default parameters.

57. The method of claim 56, wherein said upregulating expression of said polypeptide is effected by:
(i) administering said polypeptide to the subject; and/or
(ii) administering an expressible polynucleotide encoding said polypeptide to the subject.

58. An isolated polynucleotide comprising a nucleic acid sequence encoding a polypeptide having an amino acid sequence at least 85% identical to SEQ ID NO: 13, as determined using the LALIGN software of EMBnet Switzerland (http://www.ch.embnet.org/index.html) using default parameters.

59. The isolated polynucleotide of claim 58, wherein said nucleic acid sequence is as set forth in SEQ ID NO: 15.

60. The isolated polynucleotide of claim 58, wherein said nucleic acid sequence is as set forth in SEQ ID NO: 16.

61. The isolated polynucleotide of claim 58, wherein said polypeptide is as set forth in SEQ ID NO: 13.

62. The isolated polynucleotide of claim 58, wherein said polypeptide is as set forth in SEQ ID NO: 14.

63. An isolated polynucleotide as set forth in SEQ ID NO: 15.

64. An isolated polynucleotide as set forth in SEQ ID NO: 16.

65. An isolated polypeptide as set forth in SEQ ID NO: 13.

66. An isolated polypeptide as set forth in SEQ ID NO: 14.

67. A nucleic acid construct comprising the isolated polynucleotide of claim 58.

68. The nucleic acid construct of claim 67, further comprising a promoter for regulating transcription of the isolated polynucleotide in sense or antisense orientation.

69. The nucleic acid construct of claim 67, further comprising a positive and a negative selection markers for selecting for homologous recombination events.

70. A host cell comprising the nucleic acid construct of claim 67.

71. An isolated polypeptide comprising an amino acid sequence at least 85% identical to SEQ ID NO: 13, as determined using the LALIGN software of EMBnet Switzerland (http://www.ch.embnet.org/index.html) using default parameters or an active portion thereof.

72. An antibody or an antibody fragment being capable of binding a polypeptide having an amino acid sequence at least 85% identical to SEQ ID NO: 13, as determined using the LALIGN software of EMBnet Switzerland (http://www.ch.embnet.org/index.html) using default parameters.

73. An oligonucleotide hybridizable with a nucleic acid sequence encoding a polypeptide having an amino acid at least 85% identical to SEQ ID NO: 13, as determined using the LALIGN software of EMBnet Switzerland (http://www.ch.embnet.org/index.html) using default parameters.

74. A pharmaceutical composition comprising a therapeutically effective amount of a polypeptide having an amino acid sequence at least 85% identical to SEQ ID NO: 13, as determined using the LALIGN software of EMBnet Switzerland (http://www.ch.embnet.org/index.html) using default parameters and a pharmaceutically acceptable carrier or diluent.

75. A method of treating IL-7-related disease in a subject, the method comprising upregulating in the subject expression of a polypeptide having an amino acid sequence at least 85% identical to SEQ ID NO: 13 as determined using the LALIGN software of EMBnet Switzerland (http://www.ch.embnet.org/index.html) using default parameters.

76. The method of claim 75, wherein said upregulating expression of said polypeptide is effected by:
(i) administering said polypeptide to the subject; and/or
(ii) administering an expressible polynucleotide encoding said polypeptide to the subject.

77. An isolated polynucleotide comprising a nucleic acid sequence encoding a polypeptide having an amino acid sequence at least 60% identical to SEQ ID NO: 17, as determined using the LALIGN software of EMBnet Switzerland (http://www.ch.embnet.org/index.html) using default parameters.

78. The isolated polynucleotide of claim 77, wherein said nucleic acid sequence is as set forth in SEQ ID NO: 19.

79. The isolated polynucleotide of claim 77, wherein said nucleic acid sequence is as set forth in SEQ ID NO: 20.

80. The isolated polynucleotide of claim 77, wherein said polypeptide is as set forth in SEQ ID NO: 17.

81. The isolated polynucleotide of claim 77, wherein said polypeptide is as set forth in SEQ ID NO: 18.

82. An isolated polynucleotide as set forth in SEQ ID NO: 19.

83. An isolated polynucleotide as set forth in SEQ ID NO: 20.

84. An isolated polypeptide as set forth in SEQ ID NO: 17.

85. An isolated polypeptide as set forth in SEQ ID NO: 18.

86. A nucleic acid construct comprising the isolated polynucleotide of claim 77.

87. The nucleic acid construct of claim 86, further comprising a promoter for regulating transcription of the isolated polynucleotide in sense or antisense orientation.

88. The nucleic acid construct of claim 86, further comprising a positive and a negative selection markers for selecting for homologous recombination events.

89. A host cell comprising the nucleic acid construct of claim 86.

90. An isolated polypeptide comprising an amino acid sequence at least 60% identical to SEQ ID NO: 17, as determined using the LALIGN software of EMBnet Switzerland (http://www.ch.embnet.org/index.html) using default parameters or an active portion thereof.

91. An antibody or an antibody fragment being capable of binding a polypeptide having an amino acid sequence at least 60% identical to SEQ ID NO: 17, as determined using the LALIGN software of EMBnet Switzerland (http://www.ch.embnet.org/index.html) using default parameters.

92. An oligonucleotide hybridizable with a nucleic acid sequence encoding a polypeptide having an amino acid at least 60% identical to SEQ ID NO: 17, as determined using the LALIGN software of EMBnet Switzerland (http://www.ch.embnet.org/index.html) using default parameters.

93. A pharmaceutical composition comprising a therapeutically effective amount of a polypeptide having an amino acid sequence at least 60% identical to SEQ ID NO: 17, as determined using the LALIGN software of EMBnet Switzerland (http://www.ch.embnet.org/index.html) using default parameters and a pharmaceutically acceptable carrier or diluent.

94. A method of treating TNFR9-related disease in a subject, the method comprising upregulating in the subject expression of a polypeptide having an amino acid sequence at least 60% identical to SEQ ID NO: 17 as determined using the LALIGN software of EMBnet Switzerland (http://www.ch.embnet.org/index.html) using default parameters.

95. The method of claim 94, wherein said upregulating expression of said polypeptide is effected by:
(i) administering said polypeptide to the subject; and/or
(ii) administering an expressible polynucleotide encoding said polypeptide to the subject.

96. An isolated polynucleotide comprising a nucleic acid sequence encoding a polypeptide having an amino acid sequence at least 50% identical to SEQ ID NO: 25, as determined using the LALIGN software of EMBnet Switzerland (http://www.ch.embnet.org/index.html) using default parameters.

97. The isolated polynucleotide of claim 96, wherein said nucleic acid sequence is as set forth in SEQ ID NO: 27.

98. The isolated polynucleotide of claim 96, wherein said nucleic acid sequence is as set forth in SEQ ID NO: 28.

99. The isolated polynucleotide of claim 96, wherein said polypeptide is as set forth in SEQ ID NO: 25.

100. The isolated polynucleotide of claim 96, wherein said polypeptide is as set forth in SEQ ID NO: 26.

101. An isolated polynucleotide as set forth in SEQ ID NO: 27.

102. An isolated polynucleotide as set forth in SEQ ID NO: 28.

103. An isolated polypeptide as set forth in SEQ ID NO: 25.

104. An isolated polypeptide as set forth in SEQ ID NO: 26.

105. A nucleic acid construct comprising the isolated polynucleotide of claim 96.

106. The nucleic acid construct of claim 105, further comprising a promoter for regulating transcription of the isolated polynucleotide in sense or antisense orientation.

107. The nucleic acid construct of claim 105, further comprising a positive and a negative selection markers for selecting for homologous recombination events.

108. A host cell comprising the nucleic acid construct of claim 105.

109. An isolated polypeptide comprising an amino acid sequence at least 50% identical to SEQ ID NO: 25, as determined using the LALIGN software of EMBnet Switzerland (http://www.ch.embnet.org/index.html) using default parameters or an active portion thereof.

110. An antibody or an antibody fragment being capable of binding a polypeptide having an amino acid sequence at least 50% identical to SEQ ID NO: 25, as determined using the LALIGN software of EMBnet Switzerland (http://www.ch.embnet.org/index.html) using default parameters.

111. An oligonucleotide hybridizable with a nucleic acid sequence encoding a polypeptide having an amino acid at least 50% identical to SEQ ID NO: 25, as determined using the LALIGN software of EMBnet Switzerland (http://www.ch.embnet.org/index.html) using default parameters.

112. A pharmaceutical composition comprising a therapeutically effective amount of a polypeptide having an amino acid sequence at least 50% identical to SEQ ID NO: 25, as determined using the LALIGN software of EMBnet Switzerland (http://www.ch.embnet.org/index.html) using default parameters and a pharmaceutically acceptable carrier or diluent.

113. A method of treating IL-4R-related disease in a subject, the method comprising upregulating in the subject expression of a polypeptide having an amino acid sequence at least 50% identical to SEQ ID NO: 25 as determined using the LALIGN software of EMBnet Switzerland (http://www.ch.embnet.org/index.html) using default parameters.

114. The method of claim 113, wherein said upregulating expression of said polypeptide is effected by:
(i) administering said polypeptide to the subject; and/or
(ii) administering an expressible polynucleotide encoding said polypeptide to the subject.

115. An isolated polynucleotide comprising a nucleic acid sequence encoding a polypeptide having an amino acid sequence at least 50% identical to SEQ ID NO: 21, as determined using the LALIGN software of EMBnet Switzerland (http://www.ch.embnet.org/index.html) using default parameters.

116. The isolated polynucleotide of claim 115, wherein said nucleic acid sequence is as set forth in SEQ ID NO: 24.

117. The isolated polynucleotide of claim 115, wherein said nucleic acid sequence is as set forth in SEQ ID NO: 23.

118. The isolated polynucleotide of claim 115, wherein said polypeptide is as set forth in SEQ ID NO: 21.

119. The isolated polynucleotide of claim 115, wherein said polypeptide is as set forth in SEQ ID NO: 22.

120. An isolated polynucleotide as set forth in SEQ ID NO: 23.

121. An isolated polynucleotide as set forth in SEQ ID NO: 24.

122. An isolated polypeptide as set forth in SEQ ID NO: 21.

123. An isolated polypeptide as set forth in SEQ ID NO: 22.

124. A nucleic acid construct comprising the isolated polynucleotide of claim 115.

125. The nucleic acid construct of claim 124, further comprising a promoter for regulating transcription of the isolated polynucleotide in sense or antisense orientation.

126. The nucleic acid construct of claim 124, further comprising a positive and a negative selection markers for selecting for homologous recombination events.

127. A host cell comprising the nucleic acid construct of claim 124.

128. An isolated polypeptide comprising an amino acid sequence at least 50% identical to SEQ ID NO: 21, as determined using the LALIGN software of EMBnet Switzerland (http://www.ch.embnet.org/index.html) using default parameters or an active portion thereof.

129. An antibody or an antibody fragment being capable of binding a polypeptide having an amino acid sequence at least 50% identical to SEQ ID NO: 21, as determined using the LALIGN software of EMBnet Switzerland (http://www.ch.embnet.org/index.html) using default parameters.

130. An oligonucleotide hybridizable with a nucleic acid sequence encoding a polypeptide having an amino acid at least 50% identical to SEQ ID NO: 21, as determined using the LALIGN software of EMBnet Switzerland (http://www.ch.embnet.org/index.html) using default parameters.

131. A pharmaceutical composition comprising a therapeutically effective amount of a polypeptide having an amino acid sequence at least 50% identical to SEQ ID NO: 21, as determined using the LALIGN software of EMBnet Switzerland (http://www.ch.embnet.org/index.html) using default parameters and a pharmaceutically acceptable carrier or diluent.

132. A method of treating IL-4R-related disease in a subject, the method comprising upregulating in the subject expression of a polypeptide having an amino acid sequence at least 50% identical to SEQ ID NO: 21 as determined using the LALIGN software of EMBnet Switzerland (http://www.ch.embnet.org/index.html) using default parameters.

133. The method of claim 132, wherein said upregulating expression of said polypeptide is effected by:
(i) administering said polypeptide to the subject; and/or
(ii) administering an expressible polynucleotide encoding said polypeptide to the subject.

134. An isolated polynucleotide comprising a nucleic acid sequence encoding a polypeptide having an amino acid sequence at least 50% identical to SEQ ID NO: 29, as determined using the LALIGN software of EMBnet Switzerland (http://www.ch.embnet.org/index.html) using default parameters.

135. The isolated polynucleotide of claim 134, wherein said nucleic acid sequence is as set forth in SEQ ID NO: 31.

136. The isolated polynucleotide of claim 134, wherein said nucleic acid sequence is as set forth in SEQ ID NO: 32.

137. The isolated polynucleotide of claim 134, wherein said polypeptide is as set forth in SEQ ID NO: 29.

138. The isolated polynucleotide of claim 134, wherein said polypeptide is as set forth in SEQ ID NO: 30.

139. An isolated polynucleotide as set forth in SEQ ID NO: 31.

140. An isolated polynucleotide as set forth in SEQ ID NO: 32.

141. An isolated polypeptide as set forth in SEQ ID NO: 29.

142. An isolated polypeptide as set forth in SEQ ID NO: 30.

143. A nucleic acid construct comprising the isolated polynucleotide of claim 134.

144. The nucleic acid construct of claim 143, further comprising a promoter for regulating transcription of the isolated polynucleotide in sense or antisense orientation.

145. The nucleic acid construct of claim 143, further comprising a positive and a negative selection markers for selecting for homologous recombination events.

146. A host cell comprising the nucleic acid construct of claim 143.

147. An isolated polypeptide comprising an amino acid sequence at least 50% identical to SEQ ID NO: 29, as determined using the LALIGN software of EMBnet Switzerland (http://www.ch.embnet.org/index.html) using default parameters or an active portion thereof.

148. An antibody or an antibody fragment being capable of binding a polypeptide having an amino acid sequence at least 50% identical to SEQ ID NO: 29, as determined using the LALIGN software of EMBnet Switzerland (http://www.ch.embnet.org/index.html) using default parameters.

149. An oligonucleotide hybridizable with a nucleic acid sequence encoding a polypeptide having an amino acid at least 50% identical to SEQ ID NO: 29, as determined using the LALIGN software of EMBnet Switzerland (http://www.ch.embnet.org/index.html) using default parameters.

150. A pharmaceutical composition comprising a therapeutically effective amount of a polypeptide having an amino acid sequence at least 50% identical to SEQ ID NO: 29, as determined using the LALIGN software of EMBnet Switzerland (http://www.ch.embnet.org/index.html) using default parameters and a pharmaceutically acceptable carrier or diluent.

151. A method of treating TGR2-related disease in a subject, the method comprising upregulating in the subject expression of a polypeptide having an amino acid sequence at least 50% identical to SEQ ID NO: 29 as determined using the LALIGN software of EMBnet Switzerland (http://www.ch.embnet.org/index.html) using default parameters.

152. The method of claim 151, wherein said upregulating expression of said polypeptide is effected by:
(i) administering said polypeptide to the subject; and/or
(ii) administering an expressible polynucleotide encoding said polypeptide to the subject.

153. An isolated polynucleotide comprising a nucleic acid sequence encoding a polypeptide having an amino acid sequence at least 80% identical to SEQ ID NO: 33, as determined using the LALIGN software of EMBnet Switzerland (http://www.ch.embnet.org/index.html) using default parameters.

154. The isolated polynucleotide of claim 153, wherein said nucleic acid sequence is as set forth in SEQ ID NO: 35.

155. The isolated polynucleotide of claim 153, wherein said nucleic acid sequence is as set forth in SEQ ID NO: 36.

156. The isolated polynucleotide of claim 153, wherein said polypeptide is as set forth in SEQ ID NO: 33.

157. The isolated polynucleotide of claim 153, wherein said polypeptide is as set forth in SEQ ID NO: 34.

158. An isolated polynucleotide as set forth in SEQ ID NO: 35.

159. An isolated polynucleotide as set forth in SEQ ID NO: 36.

160. An isolated polypeptide as set forth in SEQ ID NO: 33.

161. An isolated polypeptide as set forth in SEQ ID NO: 34.

162. A nucleic acid construct comprising the isolated polynucleotide of claim 153.

163. The nucleic acid construct of claim 162, further comprising a promoter for regulating transcription of the isolated polynucleotide in sense or antisense orientation.

164. The nucleic acid construct of claim 162, further comprising a positive and a negative selection markers for selecting for homologous recombination events.

165. A host cell comprising the nucleic acid construct of claim 162.

166. An isolated polypeptide comprising an amino acid sequence at least 80% identical to SEQ ID NO: 33, as determined using the LALIGN software of EMBnet Switzerland (http://www.ch.embnet.org/index.html) using default parameters or an active portion thereof.

167. An antibody or an antibody fragment being capable of binding a polypeptide having an amino acid sequence at least 80% identical to SEQ ID NO: 33, as determined using the LALIGN software of EMBnet Switzerland (http://www.ch.embnet.org/index.html) using default parameters.

168. An oligonucleotide hybridizable with a nucleic acid sequence encoding a polypeptide having an amino acid at least 80% identical to SEQ ID NO: 33, as determined using the LALIGN software of EMBnet Switzerland (http://www.ch.embnet.org/index.html) using default parameters.

169. A pharmaceutical composition comprising a therapeutically effective amount of a polypeptide having an amino acid sequence at least 80% identical to SEQ ID NO: 33, as determined using the LALIGN software of EMBnet Switzerland (http://www.ch.embnet.org/index.html) using default parameters and a pharmaceutically acceptable carrier or diluent.

170. A method of treating ITAV-related disease in a subject, the method comprising upregulating in the subject expression of a polypeptide having an amino acid sequence at least 80% identical to SEQ ID NO: 33 as determined using the LALIGN software of EMBnet Switzerland (http://www.ch.embnet.org/index.html) using default parameters.

171. The method of claim 170, wherein said upregulating expression of said polypeptide is effected by:
(i) administering said polypeptide to the subject; and/or
(ii) administering an expressible polynucleotide encoding said polypeptide to the subject.

172. An isolated polynucleotide comprising a nucleic acid sequence encoding a polypeptide having an amino acid sequence at least 70% identical to SEQ ID NO: 37, as determined using the LALIGN software of EMBnet Switzerland (http://www.ch.embnet.org/index.html) using default parameters.

173. The isolated polynucleotide of claim 172, wherein said nucleic acid sequence is as set forth in SEQ ID NO: 39.

174. The isolated polynucleotide of claim 172, wherein said polypeptide is as set forth in SEQ ID NO: 37.

175. The isolated polynucleotide of claim 172, wherein said polypeptide is as set forth in SEQ ID NO: 38.

176. An isolated polynucleotide as set forth in SEQ ID NO: 39.

177. An isolated polypeptide as set forth in SEQ ID NO: 37.

178. An isolated polypeptide as set forth in SEQ ID NO: 38.

179. A nucleic acid construct comprising the isolated polynucleotide of claim 172.

180. The nucleic acid construct of claim 179, further comprising a promoter for regulating transcription of the isolated polynucleotide in sense or antisense orientation.

181. The nucleic acid construct of claim 179, further comprising a positive and a negative selection markers for selecting for homologous recombination events.

182. A host cell comprising the nucleic acid construct of claim 179.

183. An isolated polypeptide comprising an amino acid sequence at least 70% identical to SEQ ID NO: 37, as determined using the LALIGN software of EMBnet Switzerland (http://www.ch.embnet.org/index.html) using default parameters or an active portion thereof.

184. An antibody or an antibody fragment being capable of binding a polypeptide having an amino acid sequence at least 70% identical to SEQ ID NO: 37, as determined using the LALIGN software of EMBnet Switzerland (http://www.ch.embnet.org/index.html) using default parameters.

185. An oligonucleotide hybridizable with a nucleic acid sequence encoding a polypeptide having an amino acid at least 70% identical to SEQ ID NO: 37, as determined using the LALIGN software of EMBnet Switzerland (http://www.ch.embnet.org/index.html) using default parameters.

186. A pharmaceutical composition comprising a therapeutically effective amount of a polypeptide having an amino acid sequence at least 70% identical to SEQ ID NO: 37, as determined using the LALIGN software of EMBnet Switzerland (http://www.ch.embnet.org/index.html) using default parameters and a pharmaceutically acceptable carrier or diluent.

187. A method of treating IL10-R-B-related disease in a subject, the method comprising upregulating in the subject expression of a polypeptide having an amino acid sequence at least 70% identical to SEQ ID NO: 37 as determined using the LALIGN software of EMBnet Switzerland (http://www.ch.embnet.org/index.html) using default parameters.

188. The method of claim 187, wherein said upregulating expression of said polypeptide is effected by:
(i) administering said polypeptide to the subject; and/or
(ii) administering an expressible polynucleotide encoding said polypeptide to the subject.

189. An isolated polynucleotide comprising a nucleic acid sequence encoding a polypeptide having an amino acid sequence at least 80% identical to SEQ ID NO: 41, as determined using the LALIGN software of EMBnet Switzerland (http://www.ch.embnet.org/index.html) using default parameters.

190. The isolated polynucleotide of claim 189, wherein said nucleic acid sequence is as set forth in SEQ ID NO: 43.

191. The isolated polynucleotide of claim 189, wherein said nucleic acid sequence is as set forth in SEQ ID NO: 40.

192. The isolated polynucleotide of claim 189, wherein said polypeptide is as set forth in SEQ ID NO: 41.

193. The isolated polynucleotide of claim 189, wherein said polypeptide is as set forth in SEQ ID NO: 42.

194. An isolated polynucleotide as set forth in SEQ ID NO: 43.

195. An isolated polynucleotide as set forth in SEQ ID NO: 40.

196. An isolated polypeptide as set forth in SEQ ID NO: 41.

197. An isolated polypeptide as set forth in SEQ ID NO: 42.

198. A nucleic acid construct comprising the isolated polynucleotide of claim 189.

199. The nucleic acid construct of claim 189, further comprising a promoter for regulating transcription of the isolated polynucleotide in sense or antisense orientation.

200. The nucleic acid construct of claim 189, further comprising a positive and a negative selection markers for selecting for homologous recombination events.

201. A host cell comprising the nucleic acid construct of claim 198.

202. An isolated polypeptide comprising an amino acid sequence at least 80% identical to SEQ ID NO: 41, as determined using the LALIGN software of EMBnet Switzerland (http://www.ch.embnet.org/index.html) using default parameters or an active portion thereof.

203. An antibody or an antibody fragment being capable of binding a polypeptide having an amino acid sequence at least 80% identical to SEQ ID NO: 41, as determined using the LALIGN software of EMBnet Switzerland (http://www.ch.embnet.org/index.html) using default parameters.

204. An oligonucleotide hybridizable with a nucleic acid sequence encoding a polypeptide having an amino acid at least 80% identical to SEQ ID NO: 41, as determined using the LALIGN software of EMBnet Switzerland (http://www.ch.embnet.org/index.html) using default parameters.

205. A pharmaceutical composition comprising a therapeutically effective amount of a polypeptide having an amino acid sequence at least 80% identical to SEQ ID NO: 41, as determined using the LALIGN software of EMBnet Switzerland (http://www.ch.embnet.org/index.html) using default parameters and a pharmaceutically acceptable carrier or diluent.

206. A method of treating INR1-related disease in a subject, the method comprising upregulating in the subject expression of a polypeptide having an amino acid sequence at least 80% identical to SEQ ID NO: 41 as determined using the LALIGN software of EMBnet Switzerland (http://www.ch.embnet.org/index.html) using default parameters.

207. The method of claim 206, wherein said upregulating expression of said polypeptide is effected by:
(i) administering said polypeptide to the subject; and/or
(ii) administering an expressible polynucleotide encoding said polypeptide to the subject.


SEQUENCE LISTING (43 sequences)

SEQ ID NO: 1 - protein, 934 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEVGFLHSSHDVNKEASVIMLFSGLK

SEQ ID NO: 2 - protein, 24 aa, Homo sapiens
VGFLHSSHDVNKEASVIMLFSGLK

SEQ ID NO: 3 - DNA, 3042 bp, Homo sapiens
gaattccgccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagaggtgggattcctgcattcctctcatgatgtaaataaggaagccagtgtaattatgttattctcaggcttaaaataaatcattaaagctcatttatgtgtgggttttggctcatcaactc

SEQ ID NO: 4 - DNA, 118 bp, Homo sapiens
gtgggattcctgcattcctctcatgatgtaaataaggaagccagtgtaattatgttattctcaggcttaaaataaatcattaaagctcatttatgtgtgggttttggctcatcaactc

SEQ ID NO: 5 - protein, 198 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKVGVSSFPQLGVGEDRLKDSVLDNSGMQCHFQKRRLHVNKRV

SEQ ID NO: 6 - protein, 41 aa, Homo sapiens
VGVSSFPQLGVGEDRLKDSVLDNSGMQCHFQKRRLHVNKRV

SEQ ID NO: 7 - DNA, 1126 bp, Homo sapiens
ccatgtttggtaaataagtgttttggtgttgtgcaagggtctggtttcagcctgaagccatctcagagctgtctgggtctctggagactggagggacaacctagtctagagcccatttgcatgagaccaaggatcctcctgcaagagacaccatcctgagggaagagggcttctgaaccagcttgacccaataagaaattcttgggtgccgacgcggaagcagattcagagcctagagccgtgcctgcgtccgtagtttccttctagcttcttttgatttcaaatcaagacttacagggagagggagcgataaacacaaactctgcaagatgccacaaggtcctcctttgacatccccaacaaagaggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggtgggtgtgtcctcattccctcaacttggtgtgggggaagacaggctcaaagacagtgtcctggacaactcagggatgcaatgccacttccaaaagagaaggctacacgtaaacaaaagagtctgagaaatagtttctgattgttattgttaaatctttttttgtttgtttggttggttggctctcttctgcaaaggacatcaa

SEQ ID NO: 8 - DNA, 185 bp, Homo sapiens
tgggtgtgtcctcattccctcaacttggtgtgggggaagacaggctcaaagacagtgtcctggacaactcagggatgcaatgccacttccaaaagagaaggctacacgtaaacaaaagagtctgagaaatagtttctgattgttattgttaaatctttttttgtttgtttggttggttggctctc

SEQ ID NO: 9 - protein, 167 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLSSGLQKQFTFYRSNGRHTHSFHCKLSFLH

SEQ ID NO: 10 - protein, 29 aa, Homo sapiens
SSGLQKQFTFYRSNGRHTHSFHCKLSFLH

SEQ ID NO: 11 - DNA, 1789 bp, Homo sapiens
aagacgaatagtttgatttattagccaattcagataaatgtgcacgtggaagtcatagttaaatattatcgtcagtttccacgtcctgcgtttaatttggggtttgattttccaaatacaacacttaccagattaggtggacccacaggattatttttccttgaggtctcacctgagcaggtgcatgtacagcagacggagcagaaagagactgattagagaggttggagtggtagagggcgtgaccctcttaatcattcttcacttccttttttaaaagacgacttggcatcgtccaccacatccgcggcaacgcctccttggtgtcgtccgcttccaataacccagcttgcgtcctgcacacttgtggcttccgtgcacacattaacaactcatggttctagctcccagtcgccaagcgttgccaaggcgttgagagatcatctgggaagtcttttacccagaattgctttgattcaggccagctggtttttcctgcggtgattcggaaattcgcgaattcctctggtcctcatccaggtgcgcgggaagcaggtgcccaggagagaggggataatgaagattccatgctgatgatcccaaagattgaacctgcagaccaagcgcaaagtagaaactgaaagtacactgctggcggatcctacggaagttatggaaaaggcaaagcgcagagccacgccgtagtgtgtgccgccccccttgggatggatgaaactgcagtcgcggcgtgggtaagaggaaccagctgcagagatcaccctgcccaacacagactcggcaactccgcggaagaccagggtcctgggagtgactatgggcggtgagagcttgctcctgctccagttgcggtcatcatgactacgcccgcctcccgcagaccatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttgtcctcaggactacagaagcagttcacattttacagatcaaacggacgacacacacattctttccactgcaaattgtcctttctccactagaaggtatcagtttctccaaataaattgtatcaacttgagggcagacacttaattacatcttattatctcgatccccatcattgcatatccagaaagagcacataaagcgtttttcaatgcttattttagttgatggactatttgtttctttgttttgaccaataagactgaataaagataactgaggggaaaaaaattaacaactaatcaggaaataaacttttttcggatttatgaaataatttgttgacatgctctacaggagtgaccttaacatacctaatggtaactaaaactgttctctttaattacaaaattcccagcatctatcctactatgatactatctgaagataggcaccaataatacaaatgtttatccaaa

SEQ ID NO: 12 - DNA, 474 bp, Homo sapiens
tcctcaggactacagaagcagttcacattttacagatcaaacggacgacacacacattctttccactgcaaattgtcctttctccactagaaggtatcagtttctccaaataaattgtatcaacttgagggcagacacttaattacatcttattatctcgatccccatcattgcatatccagaaagagcacataaagcgtttttcaatgcttattttagttgatggactatttgtttctttgttttgaccaataagactgaataaagataactgaggggaaaaaaattaacaactaatcaggaaataaacttttttcggatttatgaaataatttgttgacatgctctacaggagtgaccttaacatacctaatggtaactaaaactgttctctttaattacaaaattcccagcatctatcctactatgatactatctgaagataggcaccaataatacaaatgtttatccaaa

SEQ ID NO: 13 - protein, 157 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLVELIIPSCMPPLLSSTSNS

SEQ ID NO: 14 - protein, 19 aa, Homo sapiens
VELIIPSCMPPLLSSTSNS

SEQ ID NO: 15 - DNA, 1541 bp, Homo sapiens
aagacgaatagtttgatttattagccaattcagataaatgtgcacgtggaagtcatagttaaatattatcgtcagtttccacgtcctgcgtttaatttggggtttgattttccaaatacaacacttaccagattaggtggacccacaggattatttttccttgaggtctcacctgagcaggtgcatgtacagcagacggagcagaaagagactgattagagaggttggagtggtagagggcgtgaccctcttaatcattcttcacttccttttttaaaagacgacttggcatcgtccaccacatccgcggcaacgcctccttggtgtcgtccgcttccaataacccagcttgcgtcctgcacacttgtggcttccgtgcacacattaacaactcatggttctagctcccagtcgccaagcgttgccaaggcgttgagagatcatctgggaagtcttttacccagaattgctttgattcaggccagctggtttttcctgcggtgattcggaaattcgcgaattcctctggtcctcatccaggtgcgcgggaagcaggtgcccaggagagaggggataatgaagattccatgctgatgatcccaaagattgaacctgcagaccaagcgcaaagtagaaactgaaagtacactgctggcggatcctacggaagttatggaaaaggcaaagcgcagagccacgccgtagtgtgtgccgccccccttgggatggatgaaactgcagtcgcggcgtgggtaagaggaaccagctgcagagatcaccctgcccaacacagactcggcaactccgcggaagaccagggtcctgggagtgactatgggcggtgagagcttgctcctgctccagttgcggtcatcatgactacgcccgcctcccgcagaccatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggtggaactgatcattccttcatgtatgcctccactgctcagctcaacaagtaactcttaataacctaccacctgttatctctgggagagggacatatgtttgccaatttctatcttcaatgcttatcacaaattttcttatatttgaaataatctgattcaaatgagaactttaacctaaaactttaattggaaagacaatcttataaaaatcttataacatattc

SEQ ID NO: 16 - DNA, 225 bp, Homo sapiens
tggaactgatcattccttcatgtatgcctccactgctcagctcaacaagtaactcttaataacctaccacctgttatctctgggagagggacatatgtttgccaatttctatcttcaatgcttatcacaaattttcttatatttgaaataatctgattcaaatgagaactttaacctaaaactttaattggaaagacaatcttataaaaatcttataacatattc

SEQ ID NO: 17 - protein, 153 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNIRVADEWNHDSQEKY

SEQ ID NO: 18 - protein, 15 aa, Homo sapiens
IRVADEWNHDSQEKY

SEQ ID NO: 19 - DNA, 946 bp, Homo sapiens
gagaccaaggagtggaaagttctccggcagccctgagatctcaagagtgacatttgtgagaccagctaatttgattaaaattctcttggaatcagctttgctagtatcatacctgtgccagatttcatcatgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaacatcagagtggctgacgaatggaatcatgattcacaagaaaagtattgactattttctcggacttagctgaattctgtctttggaaagtggcttttttaaaaagctgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaacgtaagattaacataatcatattacagctctggca

SEQ ID NO: 20 - DNA, 104 bp, Homo sapiens
catcagagtggctgacgaatggaatcatgattcacaagaaaagtattgactattttctcggacttagctgaattctgtctttggaaagtggcttttttaaaaag

SEQ ID NO: 21 - protein, 131 aa, Homo sapiens [sequence omitted]

SEQ ID NO: 22 - protein, 10 aa, Homo sapiens
LPPLKRSWSQ

SEQ ID NO: 23 - DNA, 4848 bp, Homo sapiens
tgcagtgcccgacagattgtactagttactgattgaagggctgttttactatccaaatgtggctggagtaggagttgggtaaacatttattgaagaatgtgcaaccactctcacttggaagccgggctgttaggaaggggaggaggattccagtcgcccagccctcccccaccaaacgcaactgccccggcgcaaaagaggccgcggaggccaggcaggagcaggtcctggaggcctggtcggcgtgggcgttttattccgagaccaaggggatccactgcagagttctccgctgggcgtgacctcgggctacggcgtgggaggaagcgcgcggcaagacacccagcgaggtgctggggtcgcccccaggagaggacggcggctcggactgtccggcggcggcggcggggacagcgacaggggcgcgaggtggccgggacccgggccgggcgcgccgggcggggcggcgcatgcaaatctgccgggcgccggggcggggagcaggaagccggggcgggctgggtctccgcgcccaggaaagccccgcgcggcgcgggccagggaagggccacccaggggtcccccacttcccgcttgggcgcccggacggcgaatggagcaggggcgcgcagataattaaagatttacacacagctggaagaaatcatagagaagccgggcgtggtggctcatgcctataatcccagcacttttggaggctgaggcgggcagatcacttgagatcaggagttcgagaccagcctggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtcctcccacctttgaaacggagctggtcgcagtagaccaccaagcccccttcagcccagctgtttccacccctgaacttaagtgcccaggaaggcgtattgagatgaggtgtgcttgctggaaggcatgcctgctgctgattgaaaaccgaactgggaacagtccttccattctgtgtccactggtcagctgctgcggctttggatggtcttgaccgtggaaggctgaccttcttctggtacccggagtccctgcaggaatcccccttgagcttgctgggctgtggtgacaggagtttaaaacatgcgttgtattccagtgatgcatgatatgacatgcatcacaggaataaaaacctgaggtctcatggatatgattgcttcaaaggagaccaagttttaaaacagatgaatcaaaataaagaaaaatactcagtaaatcatcataaagtacagagatgtggccaaaggtgtgaaggatgcagctgtaaaagctgaagtttgaggccgggtgtggtggttcatgcctataatcccagcactttgggaggccgagcccagcggatcaccggaggtcaggagttcgagaccagcctggacaacatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagccccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacatggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctgtgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctctttaaaaattatgtattaaccaaacatacctccagac

SEQ ID NO: 24 - DNA, 605 bp, Homo sapiens
catgtcctcccacctttgaaacggagctggtcgcagtagaccaccaagcccccttcagcccagctgtttccacccctgaacttaagtgcccaggaaggcgtattgagatgaggtgtgcttgctggaaggcatgcctgctgctgattgaaaaccgaactgggaacagtccttccattctgtgtccactggtcagctgctgcggctttggatggtcttgaccgtggaaggctgaccttcttctggtacccggagtccctgcaggaatcccccttgagcttgctgggctgtggtgacaggagtttaaaacatgcgttgtattccagtgatgcatgatatgacatgcatcacaggaataaaaacctgaggtctcatggatatgattgcttcaaaggagaccaagttttaaaacagatgaatcaaaataaagaaaaatactcagtaaatcatcataaagtacagagatgtggccaaaggtgtgaaggatgcagctgtaaaagctgaagtttgaggccgggtgtggtggttcatgcctataatcccagcactttgggaggccgagcccagcggatcaccggaggtcaggagttcgagaccagcctggacaa

SEQ ID NO: 25 - protein, 229 aa, Homo sapiens [sequence omitted]

SEQ ID NO: 26 - protein, 6 aa, Homo sapiens
CEYQEA

SEQ ID NO: 27 - DNA, 1541 bp, Homo sapiens
tgcagtgcccgacagattgtactagttactgattgaagggctgttttactatccaaatgtggctggagtaggagttgggtaaacatttattgaagaatgtgcaaccactctcacttggaagccgggctgttaggaaggggaggaggattccagtcgcccagccctcccccaccaaacgcaactgccccggcgcaaaagaggccgcggaggccaggcaggagcaggtcctggaggcctggtcggcgtgggcgttttattccgagaccaaggggatccactgcagagttctccgctgggcgtgacctcgggctacggcgtgggaggaagcgcgcggcaagacacccagcgaggtgctggggtcgcccccaggagaggacggcggctcggactgtccggcggcggcggcggggacagcgacaggggcgcgaggtggccgggacccgggccgggcgcgccgggcggggcggcgcatgcaaatctgccgggcgccggggcggggagcaggaagccggggcgggctgggtctccgcgcccaggaaagccccgcgcggcgcgggccagggaagggccacccaggggtcccccacttcccgcttgggcgcccggacggcgaatggagcaggggcgcgcagataattaaagatttacacacagctggaagaaatcatagagaagccgggcgtggtggctcatgcctataatcccagcacttttggaggctgaggcgggcagatcacttgagatcaggagttcgagaccagcctggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactgtgagtatcaagaggcctaagcaatggtaatctccactctccattcttcccctgtggccagacacttcccctggctgagtctctgggc

SEQ ID NO: 28 - DNA, 88 bp, Homo sapiens
gtgagtatcaagaggcctaagcaatggtaatctccactctccattcttcccctgtggccagacacttcccctggctgagtctctgggc

SEQ ID NO: 29 - protein, 176 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEGEFSSLKGVGPEICANFLYPWSAVS

SEQ ID NO: 30 - protein, 25 aa, Homo sapiens
GEFSSLKGVGPEICANFLYPWSAVS

SEQ ID NO: 31 - DNA, 1850 bp, Homo sapiens
acctaaagaaaaacattttacaacttgacagtgtatgcacatacatacatgcatatagacacaactgaagcacaaatttaatgaagtagaatttaccgttactattttatttgggaaagaaatgtgctcgcgactcaatagattggagtattcactcctggatctcaacttgcaatttgaaaacgcatctctaaagcacctaggagcaatctgaagaaagctgaggggaggcggcagatgttctgatctactagggaaaacgtggacgttttctgttgttactttgtgaactgtgtgcacttagtcattcttgagtaaatacttggagcgaggaactcctgagtggtgtgggagggcggtgaggggcagctgaaagtcggccaaagctctcggaggggctggtctaggaaacatgattggcagctacgagagagctaggggctggacgtcgaggagagggagaaggctctcgggcggagagaggtcctgcccagctgttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaaggtgagttttcttctcttaagggtgtgggacctgagatctgtgccaattttttgtatccttggtctgcagtgtcatagagcacattcctcctgtggtggattgcatacagtggattaggagctcattcagctggtggaaagaggggcttggggagtagcagggtttgttctggttctcatcaaatatggttgactggggcaaacattattatttgtctttgacaaatagtttctttcacctagagcagtgtttctcaaagtgcggccccttgagcagccagcatcagtatcacctgggaacctgttataaatgcagattctcaggccccactaaatgagaaacatagagggtgaaccccagctatctgtattttaacaagccctcccagtaattctgtgcagctaaaatttggtaactattgttctaaagatttggatggggttgtttaatcttggaggaggactttctttataactgatgttgtttcttgtacatagtcccaggatttgtctttagggtacttgtcatcgatcccatttgagagacactttgcaatacagag

SEQ ID NO: 32 - DNA, 564 bp, Homo sapiens
aggtgagttttcttctcttaagggtgtgggacctgagatctgtgccaattttttgtatccttggtctgcagtgtcatagagcacattcctcctgtggtggattgcatacagtggattaggagctcattcagctggtggaaagaggggcttggggagtagcagggtttgttctggttctcatcaaatatggttgactggggcaaacattattatttgtctttgacaaatagtttctttcacctagagcagtgtttctcaaagtgcggccccttgagcagccagcatcagtatcacctgggaacctgttataaatgcagattctcaggccccactaaatgagaaacatagagggtgaaccccagctatctgtattttaacaagccctcccagtaattctgtgcagctaaaatttggtaactattgttctaaagatttggatggggttgtttaatcttggaggaggactttctttataactgatgttgtttcttgtacatagtcccaggatttgtctttagggtacttgtcatcgatcccatttgagagacactttgcaatacagag

SEQ ID NO: 33 - protein, 815 aa, Homo sapiens
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHIFLPIPNWEHKENPETEEDVGPVVQHIYEVCSC

SEQ ID NO: 34 - protein, 4 aa, Homo sapiens
VCSC

SEQ ID NO: 35 - DNA, 3379 bp, Homo sapiens
gataaaaagctttcctcatttttaaacaacagtcgcacggaagttcccggcgggacaagggaacgtgggtgcccttgctactcccgtggacgcgggtagattgggacgctggaccgtatctccccgcccccgcccccacgcctcctcaggtgctcagcctgaggccttcgtccaggagcgctgccgctgacccaggctcaggagctgggggcccctgcacagacgcccaggtctcgggacaggcggcgactgcactcacggaagtacgctgagctctcccctgtagaagggcgcctctcctcccccacttcctcctccagctccacagcagcctcccgggccggctcctcctccttccaggtctcctcccagtgccgccgcggctctcaggcctgaggtgcggcgctcaccccggcagtccccagcctcagacgctgcgtggagcggcggagccggagggaagcaaaggaccgtctgcgctgctgtccccgccccgcgcgctctgcgcccctcgtccctggcggtcgctccgaagctcagccctcttgcctgccccggagctgtcccgggctagccgagaagagagcggccggcaagtttgggcgcgcgcaggcggcgggccgcgggcactgggcgcctcgctggggcggggggaggtggctaccgctcccggcttggcgtcccgcgcgcacttcggcgatggcttttccgccgcggcgacggctgcgcctcggtccccgcggcctcccgcttcttctctcgggactcctgctacctctgtgccgcgccttcaacctagacgtggacagtcctgccgagtactctggccccgagggaagttacttcggcttcgccgtggatttcttcgtgcccagcgcgtcttcccggatgtttcttctcgtgggagctcccaaagcaaacaccacccagcctgggattgtggaaggagggcaggtcctcaaatgtgactggtcttctacccgccggtgccagccaattgaatttgatgcaacaggcaatagagattatgccaaggatgatccattggaatttaagtcccatcagtggtttggagcatctgtgaggtcgaaacaggataaaattttggcctgtgccccattgtaccattggagaactgagatgaaacaggagcgagagcctgttggaacatgctttcttcaagatggaacaaagactgttgagtatgctccatgtagatcacaagatattgatgctgatggacagggattttgtcaaggaggattcagcattgattttactaaagctgacagagtacttcttggtggtcctggtagcttttattggcaaggtcagcttatttcggatcaagtggcagaaatcgtatctaaatacgaccccaatgtttacagcatcaagtataataaccaattagcaactcggactgcacaagctatttttgatgacagctatttgggttattctgtggctgtcggagatttcaatggtgatggcatagatgactttgtttcaggagttccaagagcagcaaggactttgggaatggtttatatttatgatgggaagaacatgtcctccttatacaattttactggcgagcagatggctgcatatttcggattttctgtagctgccactgacattaatggagatgattatgcagatgtgtttattggagcacctctcttcatggatcgtggctctgatggcaaactccaagaggtggggcaggtctcagtgtctctacagagagcttcaggagacttccagacgacaaagctgaatggatttgaggtctttgcacggtttggcagtgccatagctcctttgggagatctggaccaggatggtttcaatgatattgcaattgctgctccatatgggggtgaagataaaaaaggaattgtttatatcttcaatggaagatcaacaggcttgaacgcagtcccatctcaaatccttgaagggcagtgggctgctcgaagcatgccaccaagctttggctattcaatgaaaggagccacagatatagacaaaaatggatatccagacttaattgtaggagcttttggtgtagatcgagctatcttatacagggccagaccagttatcactgtaaatgctggtcttgaagtgtaccctagcattttaaatcaagacaataaaacctgctcactgcctggaacagctctcaaagtttcctgttttaatgttaggttctgcttaaaggcagatggcaaaggagtacttcccaggaaacttaatttccaggtggaacttcttttggataaactcaagcaaaagggagcaattcgacgagcactgtttctctacagcaggtccccaagtcactccaagaacatgactatttcaagggggggactgatgcagtgtgaggaattgatagcgtatctgcgggatgaatctgaatttagagacaaactcactccaattactatttttatggaatatcggttggattatagaacagctgctgatacaacaggcttgcaacccattcttaaccagttcacgcctgctaacattagtcgacaggctcacattctacttgactgtggtgaagacaatgtctgtaaacccaagctggaagtttctgtagatagtgatcaaaagaagatctatattggggatgacaaccctctgacattgattgttaaggctcagaatcaaggagaaggtgcctacgaagctgagctcatcgtttccattccactgcaggctgatttcatcggggttgtccgaaacaatgaagccttagcaagactttcctgtgcatttaagacagaaaaccaaactcgccaggtggtatgtgaccttggaaacccaatgaaggctggaactcaactcttagctggtcttcgtttcagtgtgcaccagcagtcagagatggatacttctgtgaaatttgacttacaaatccaaagctcaaatctatttgacaaagtaagcccagttgtatctcacaaagttgatcttgctgttttagctgcagttgagataagaggagtctcgagtcctgatcatatctttcttccgattccaaactgggagcacaaggagaaccctgagactgaagaagatgttgggccagttgttcagcacatctatgaggtttgcagttgttagattttactcaaacctcgtgagcaagccaacgaagagaggaacaactaagctactttaaaaaaaaaattctatgtaatttttatgtaaactctacattggttaagtatgtgtcagagatttctttgaatattttccctatacataaattcatttttatttgacaaatagacttgtttaaataaagcagtttatataatttgttgtttaaaataaattagttctacttgaataa

SEQ ID NO: 36 - DNA, 247 bp, Homo sapiens
gtttgcagttgttagattttactcaaacctcgtgagcaagccaacgaagagaggaacaactaagctactttaaaaaaaaaattctatgtaatttttatgtaaactctacattggttaagtatgtgtcagagatttctttgaatattttccctatacataaattcatttttatttgacaaatagacttgtttaaataaagcagtttatataatttgttgtttaaaataaattagttctacttgaataa

SEQ ID NO: 37 - protein, 222 aa, Homo sapiens
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDVFGPSSS

SEQ ID NO: 38 - protein, 7 aa, Homo sapiens
VFGPSSS

SEQ ID NO: 39 - DNA, 1806 bp, Homo sapiens
cccgcccatctccgctggttcccggaagccgccgcggacaagctctcccgggcgcgggcgggggtcgtgtgcttggaggaagccgcggaacccccagcgtccgtccatggcgtggagccttgggagctggctgggtggctgcctgctggtgtcagcattgggaatggtaccacctcccgaaaatgtcagaatgaattctgttaatttcaagaacattctacagtgggagtcacctgcttttgccaaagggaacctgactttcacagctcagtacctaagttataggatattccaagataaatgcatgaatactaccttgacggaatgtgatttctcaagtctttccaagtatggtgaccacaccttgagagtcagggctgaatttgcagatgagcattcagactgggtaaacatcaccttctgtcctgtggatgacaccattattggaccccctggaatgcaagtagaagtacttgctgattctttacatatgcgtttcttagcccctaaaattgagaatgaatacgaaacttggactatgaagaatgtgtataactcatggacttataatgtgcaatactggaaaaacggtactgatgaaaagtttcaaattactccccagtatgactttgaggtcctcagaaacctggagccatggacaacttattgtgttcaagttcgagggtttcttcctgatcggaacaaagctggggaatggagtgagcctgtctgtgagcaaacaacccatgacgtttttgggccatcctcatcataacacacttctgtttttctcctttccattgtcggatgagaatgatgtttttgacaagctaagtgtcattgcagaagactctgagagcggcaagcagaatcctggtgacagctgcagcctcgggaccccgcctgggcaggggccccaaagctaggctctgagaaggaaacacactcggctgggcacagtgacgtactccatctcacatctgcctcagtgagggatcagggcagcaaacaagggccaagaccatctgagccagccccacatctagaactcccagaccctggacttagccaccagagagctacattttaaaggctgtcttggcaaaaatactccatttgggaactcactgccttataaaggctttcatgatgttttcagaagttggccactgagagtgtaattttcagccttttatatcactaaaataagatcatgttttaattgtgagaaacagggccgagcacagtggctcacgcctgtaataccagcaccttagaggtcgaggcaggcggatcacttgaggtcaggagttcaagaccagcctggccaatatggtgaaacccagtctctactaaaaatacaaaaattagctaggcatgatggcgcatgcctataatcccagctactcgagtgcctgaggcaggagaattgcatgaacccgggaggaggaggaggaggttgcagtgagccgagatagcggcactgcactccagcctgggtgacaaagtgagactccatctcaaaaaaaaaaaaaaaaaattgtgagaaacagaaatacttaaaatgaggaataagaatggagatgttacatctggtagatgtaacattctaccagattatggatggactgatctgaaaatcaacctcaactcaagggtggtcagctcaatgctacacagagcacggacttttggattctttgcagtactttgaatttatttttctacctatatatgttttatatgctgctggtgctccattaaagttttactctgtgttgcactatatgtgttcatgataaaaaa

SEQ ID NO: 40 - DNA, 65 bp, Homo sapiens
tcagaatcttttattgtcttttttaaaaatgtagctagacataataaaagtaattctatactgta

SEQ ID NO: 41 - protein, 441 aa, Homo sapiens
MVVLLGATTLVLVAVAPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGQNLLLSFLKM

SEQ ID NO: 42 - protein, 10 aa, Homo sapiens
QNLLLSFLKM

SEQ ID NO: 43 - DNA, 1512 bp, Homo sapiens
agaagaggcggcgcgtgcgtagaggggcggtgagagctaagaggggcagcgcgtgtgcagaggggcggtgtgacttaggacggggcgatggcggctgagaggagctgcgcgtgcgcgaacatgtaactggtgggatctgcggcggctcccagatgatggtcgtcctcctgggcgcgacgaccctagtgctcgtcgccgtggcgccatgggtgttgtccgcagccgcaggtggaaaaaatctaaaatctcctcaaaaagtagaggtcgacatcatagatgacaactttatcctgaggtggaacaggagcgatgagtctgtcgggaatgtgactttttcattcgattatcaaaaaactgggatggataattggataaaattgtctgggtgtcagaatattactagtaccaaatgcaacttttcttcactcaagctgaatgtttatgaagaaattaaattgcgtataagagcagaaaaagaaaacacttcttcatggtatgaggttgactcatttacaccatttcgcaaagctcagattggtcctccagaagtacatttagaagctgaagataaggcaatagtgatacacatctctcctggaacaaaagatagtgttatgtgggctttggatggtttaagctttacatatagcttagttatctggaaaaactcttcaggtgtagaagaaaggattgaaaatatttattccagacataaaatttataaactctcaccagagactacttattgtctaaaagttaaagcagcactacttacgtcatggaaaattggtgtctatagtccagtacattgtataaagaccacagttgaaaatgaactacctccaccagaaaatatagaagtcagtgtccaaaatcagaactatgttcttaaatgggattatacatatgcaaacatgacctttcaagttcagtggctccacgcctttttaaaaaggaatcctggaaaccatttgtataaatggaaacaaatacctgactgtgaaaatgtcaaaactacccagtgtgtctttcctcaaaacgttttccaaaaaggaatttaccttctccgcgtacaagcatctgatggaaataacacatctttttggtctgaagagataaagtttgatactgaaatacaagctttcctacttcctccagtctttaacattagatcccttagtgattcattccatatctatatcggtgctccaaaacagtctggaaacacgcctgtgatccaggattatccactgatttatgaaattattttttgggaaaacacttcaaatgctgagagaaaaattatcgagaaaaaaactgatgttacagttcctaatttgaaaccactgactgtatattgtgtgaaagccagagcacacaccatggatgaaaagctgaataaaagcagtgtttttagtgacgctgtatgtgagaaaacaaaaccaggtcagaatcttttattgtcttttttaaaaatgtagctagacataataaaagtaattctatactgta